(19) United States
(12) Patent Application Publication
(10) Pub. No.: US 2022/0401489 A1
(43) Pub. Date: Dec. 22, 2022

(54) COMPOSITIONS AND METHODS FOR TREATING SICKLE CELL DISEASE

(71) Applicant: Syros Pharmaceuticals, Inc., Cambridge, MA (US)

(72) Inventor: Jeffrey R. Shearstone, Framingham, MA (US)

(21) Appl. No.: 17/774,779

(22) PCT Filed: Nov. 6, 2020
(86) PCT No.: PCT/US2020/059535

§ 371 Date: May 5, 2022

Related U.S. Application Data
(60) Provisional application No. 62/931,692, filed on Nov. 6, 2019.

(51) Int. Cl. A61K 35/28; A61K 9/00

(52) U.S. Cl. CPC A61K 35/28; A61K 9/0019; A61K 2035/124

(57) ABSTRACT

The present invention features compositions and methods useful in inhibiting the expression of NFIX within a cell and thereby treating patients suffering from a hemoglobinopathy such as sickle cell disease or β-thalassemia

### CROSS-REFERENCE TO RELATED APPLICATIONS

This application claims the benefit of and priority to U.S. Provisional Application No. 62/931,692, filed Nov. 6, 2019, the disclosure of which is hereby incorporated by reference in its entirety for all purposes.

### BACKGROUND

Sickle cell disease (SCD) occurs in patients who have inherited, in both copies of the gene encoding the adult hemoglobin beta chain (HBB), a mutation that results in the substitution of valine for glutamic acid at the sixth amino acid position of the encoded protein. This single substitution profoundly alters the conformation and function of the hemoglobin molecule, and the red blood cells expressing such altered hemoglobin become deformed and rigid. Because the deformed red blood cells die much more quickly than unaffected red blood cells, patients become anemic, experience episodic pain, and are at risk of serious conditions such as stroke, pulmonary hypertension, and vision loss. Although distinct from SCD, β-thalassemia is also an inherited blood disorder. The mutations that occur in β-thalassemias are much more complex than the mutation underlying SCD, but the resulting disease is associated with reduced synthesis of the beta chains of hemoglobin and affected patients have varying symptoms, from asymptomatic disease to severe anemia.

### SUMMARY OF THE INVENTION

The present invention features, inter alia, methods of treating a hemoglobinopathy in a patient in need thereof. The methods can be carried out by administering to the patient an effective amount of a pharmaceutically acceptable composition comprising a genetically modified cell that comprises a nucleic acid construct that inhibits the expression of NFIX within the cell. The hemoglobinopathy can be a β-thalassemia or sickle cell disease (SCD), as described further herein, and the cell can be a hematopoietic stem cell, a hematopoietic progenitor cell, or an erythroblast at any stage of development prior to enucleation. The cell can be autologous to the patient. In any of these embodiments, the nucleic acid construct can be or can include an antisense oligonucleotide (ASO), short hairpin RNA (shRNA), small inhibitory RNA (siRNA), microRNA (miRNA) or morpholino oligomer, as described further below, that inhibits the expression of NFIX. For example, the nucleic acid construct can include a sequence that base pairs (through complementarity) with a sequence within or transcribed from the region of NFIX that encodes a DNA-binding domain or a sequence within the NFIX promoter/regulatory region (as shown in FIG. 10). The DNA-binding domain has the sequence:

(SEQ ID NO:_)
KQKWASRLLAKLRKDIRPEFREDFVLTITGKKPPCCVLSNPDQKGKIR
RIDCLRQADKVWRLDLVMVILFKGIPLESTDGERLYKSPQCSNPGLCV
QPHHIGV.

In some embodiments, the shRNA or antisense nucleic acid sequence base pairs (through complementarity) with a sequence within an exon (e.g., Exon 2 and/or Exon 3) of NFIX. More specifically, a nucleic acid construct within the cells of the invention and useful in the present methods can include an shRNA comprising a sequence provided in Table 1, below, or a sequence at least 80% (e.g., at least 85%, at least 90%, or at least 95%) identical thereto, optionally wherein the shRNA terminates at the residue immediately prior to the terminal poly-T sequence or comprises only the internally complementary sequences. In one embodiment, the nucleic acid construct comprises an shRNA comprising

(SEQ ID NO:_)
CCGGACATTGGAGTCACAATCAAAGCTCGAGCTTTGATTGTGACTCCA
ATGTTTTTTG
or
(SEQ ID NO:_)
CCGGGGAATCCGGACAATCAGATAGCTCGAGCTATCTGATTGTCCGGA
TTCCTTTTTG.

As one of ordinary skill in the art would understand, nucleic acid constructs can be made or purchased containing “T” nucleotides (as shown, e.g., in the sequences above), however upon expression in a cell, such nucleotides are replaced with uracil. It is well understood that where DNA includes “T”, an RNA transcribed therefrom includes “U.” The genetically modified cells of the invention can include the components of a nuclease-editing system (e.g., CRISPR) or a transposon. For example, the genetically modified cell can include a guide RNA that base pairs with a sequence within the region of NFIX that encodes a DNA-binding domain or within the NFIX promoter/regulatory region (as shown in FIG. 10). As noted, the genetically modified cell can be non-naturally occurring and characterized by a level of NFIX expression that is decreased relative to that of a comparable, non-genetically modified cell of the same type. In any of these embodiments, the cell can be a hematopoietic stem cell, a hematopoietic progenitor cell, or an erythroblast at any stage of development prior to enucleation. As described elsewhere herein, the cell can include an shRNA or antisense nucleic acid sequence that inhibits the expression of NFIX.

The invention also features pharmaceutically acceptable compositions that include a non-naturally occurring, genetically modified cell, as described herein, and such compositions can be formulated for parenteral administration (e.g., intravenous administration) to a patient suffering from a hemoglobinopathy.

The invention also features kits that include a composition described herein (e.g., a genetically modified cell in which NFIX expression is inhibited), optionally within a pharmaceutically acceptable composition, and instructions for use (e.g., written material).

### BRIEF DESCRIPTION OF THE DRAWINGS

FIG. 1 is a schematic illustrating the effect of mutated adult beta globin on RBCs.

FIG. 2 provides three representative nucleic acid sequences of human NFIX splice variants, any of which can be a target of a nucleic acid construct as described herein, and the sequences of the proteins encoded thereby. Isoform 1 (SEQ ID NO: ______) is encoded by transcript variant 1 (SEQ ID NO: ______); isoform 2 (SEQ ID NO: ______) is encoded by transcript variant 2 (SEQ ID NO: ______); and isoform 3 (SEQ ID NO: ______) is encoded by transcript variant 3 (SEQ ID NO: ______).

FIG. 3 is a Table providing information regarding 14 known splice variants of NFIX, any of which can be a target of a nucleic acid construct as described herein.

FIG. 4 provides an illustration of the genes that encode hemoglobin during embryonic development (HBE), fetal development (HBG1, HBG2), and adulthood (HBD, HBB) and graphical representations of their mRNA (% of total β-like globins) and corresponding protein levels (HbF indicating fetal hemoglobin and HbA indicating adult hemoglobin) before and after fetal-to-adult hemoglobin switching.

FIGS. 5A-5D are a collection of results concerning beta-like globin expression and chromatin accessibility in the seven discrete BM and CB cell populations described in the Examples. FIG. 5A illustrates the sorting schema for primary erythroblasts derived from CD34+ bone marrow and cord blood cells; based on CD36 and GYPA expression, the cells from each source were sorted into seven populations (“Pop 1”-“Pop 7”). FIG. 5B shows the morphology of representative cells in each population following Giemsa-benzimide staining. FIG. 5C provides the relative levels of each beta-like globin mRNA in the sorted cell populations (transcripts per million (%)), and FIG. 5D illustrates chromatin accessibility at the beta-like globin locus in the sorted cell populations.

FIGS. 6A-6E are a collection of chromatin accessibility data that suggest NFIX as an HbF repressor. FIG. 6A illustrates hierarchical clustering and FIG. 6B is a principal component analysis of ATAC-seq peaks from CB- and BM-derived populations of cells. FIG. 6C illustrates NFI factor DNA binding motif enrichment under chromatin accessibility peaks. FIG. 6D shows the results of a transcription factor footprinting analysis of NFI motifs within chromatin accessibility peaks, and FIG. 6E shows chromatin accessibility at the NFIX promoter in the BM- and CB-derived cell populations.

FIG. 7 is a schematic illustration of events over the 14-day in vitro study of lentiviral-mediated NFIX knockdowns in primary BM cells.

FIG. 8 is a map of the TRC1.5 vector construct used to make viral supernatants that deliver NFIX-targeted shRNAs to cells.

FIGS. 9A-9H are a collection of data from experiments in which NFIX was knocked down with lentiviral-delivered shRNAs. FIG. 9A is a bar graph illustrating NFIX mRNA levels (rel ACTB) at Days 4, 7, and 10 following exposure to a control shRNA (shCtrl), shNFIX #1, or shNFIX #2. FIG. 9B is a line graph illustrating induction of HBG mRNA (as % of total β-like globin) for 14 days following exposures to a control shRNA (shCtrl), an shRNA inhibiting BCL11A (shBCL11A), an shRNA inhibiting ZBTB7A (shZBTB7A), shNFIX #1, or shNFIX #2. FIG. 9C illustrates HbF protein levels at Day 14 following exposure to a control shRNA (shCtrl), shNFIX #1, or shNFIX #2. In FIG. 9D, F-cells (Day 10) were evaluated in erythroblasts derived from human CD34+ bone marrow cells transduced with a control shRNA (shCtrl), shNFIX #1, or shNFIX #2. FIG. 9E is a bar graph illustrating HBG mRNA, HbF, and F-cells (%) in HUDEP-2 cells transduced with a control shRNA (shCtrl), shNFIX #1, or shNFIX #2 and differentiated for 7 days. FIG. 9F illustrates chromatin accessibility at the beta-globin locus at Day 7 in primary erythroblasts transduced with a control shRNA (shCtrl), shNFIX #1, or shNFIX #2. FIG. 9G is a line graph illustrating DNA methylation (%) at the HBG promoter in primary erythroblasts transduced with a control shRNA (shCtrl), shNFIX #1, or shNFIX #2. FIG. 9H illustrates erythroid differentiation in primary erythroblasts transduced with a control shRNA (shCtrl), shNFIX #1, or shNFIX #2.

FIG. 10 shows chromatin accessibility at the NFIX promoter and includes DNA sequence within the NFIX promoter/regulatory region we believe can be targeted as described herein to inhibit NFIX expression.

FIG. 11 is an illustration of nucleic acid sequences that can be incorporated into a nucleic acid construct as described herein for inhibiting the expression of NFIX (SEQ ID NOs ______-______).

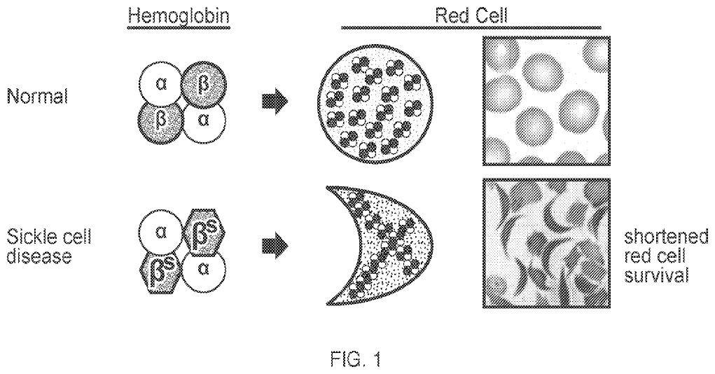

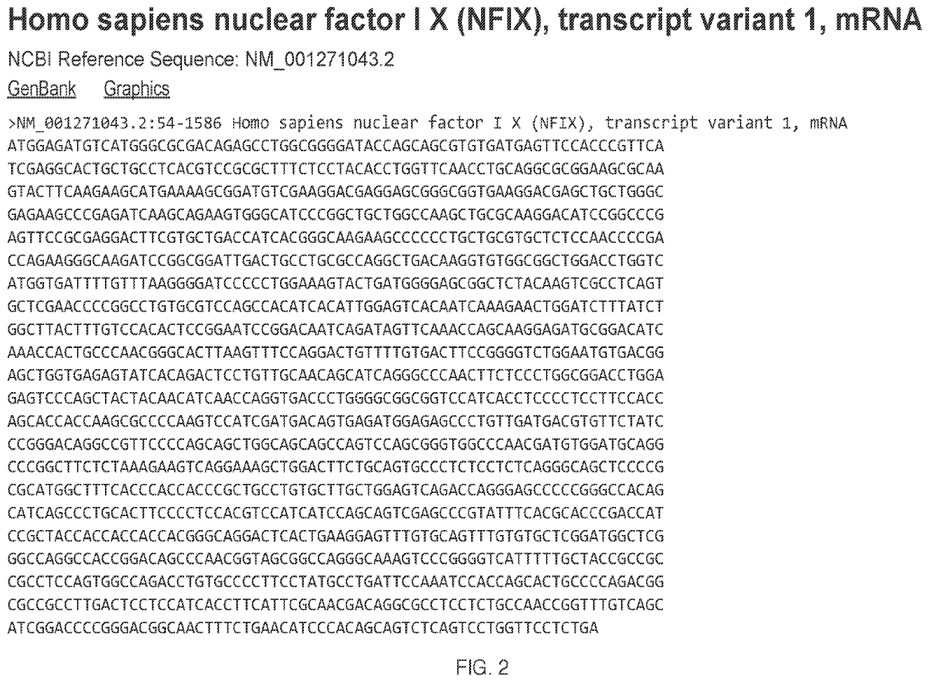

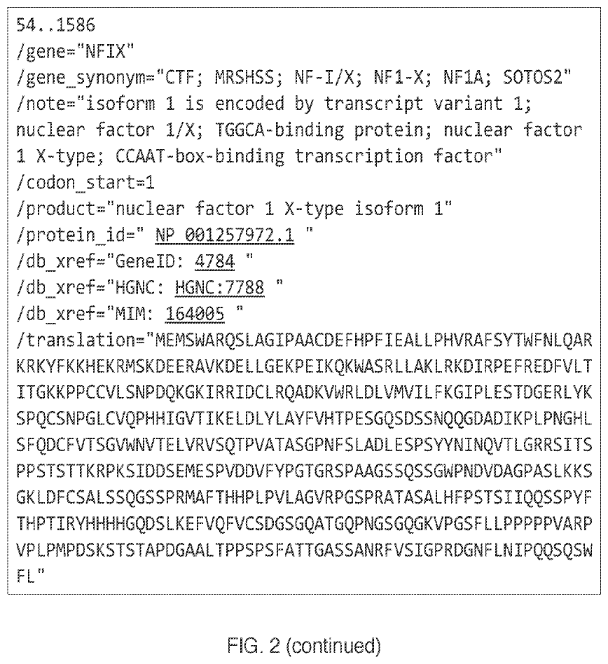

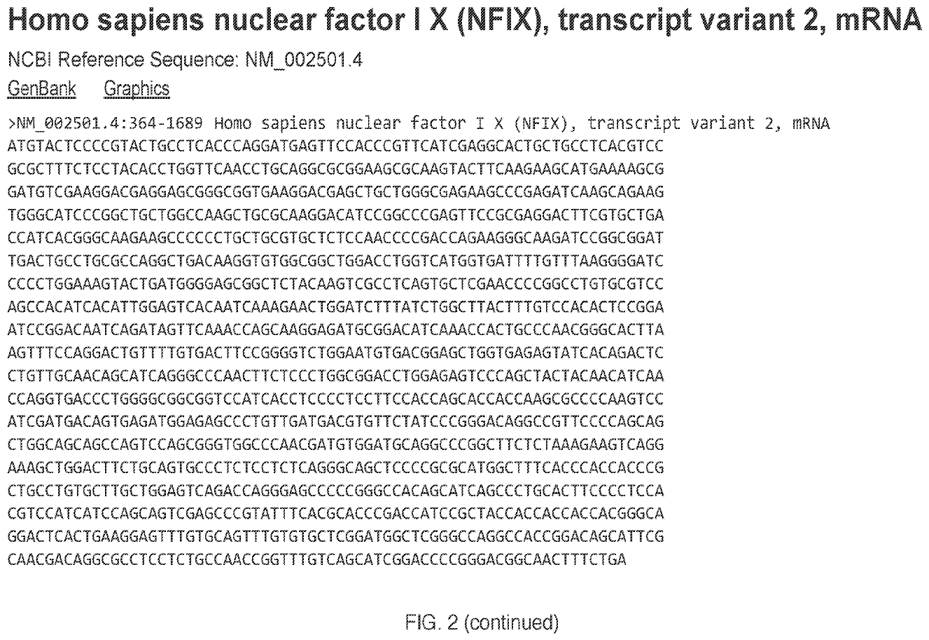

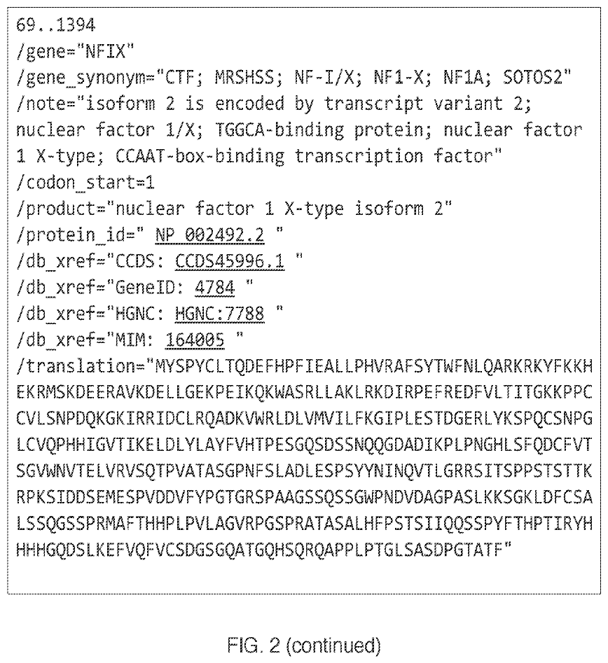

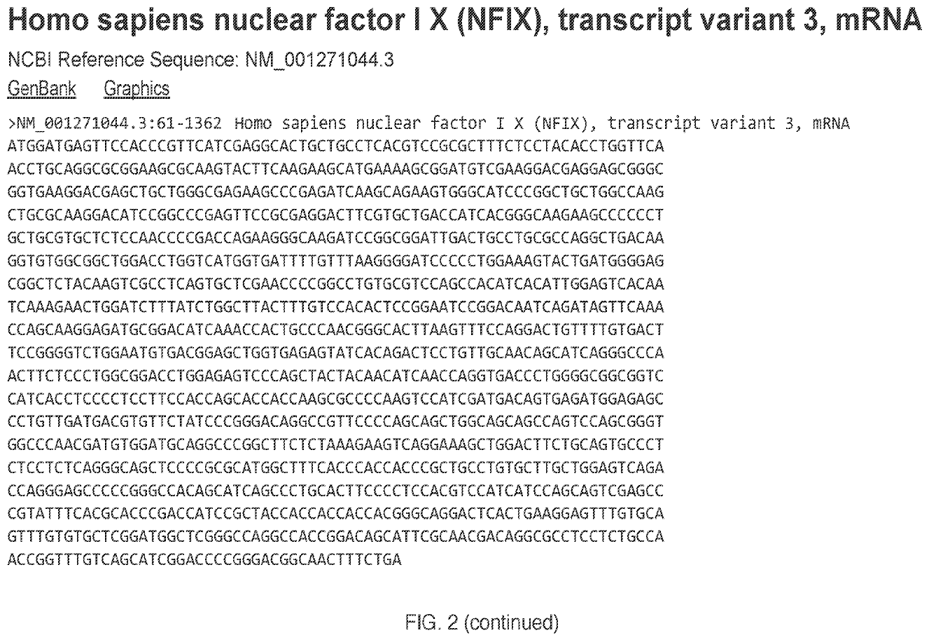

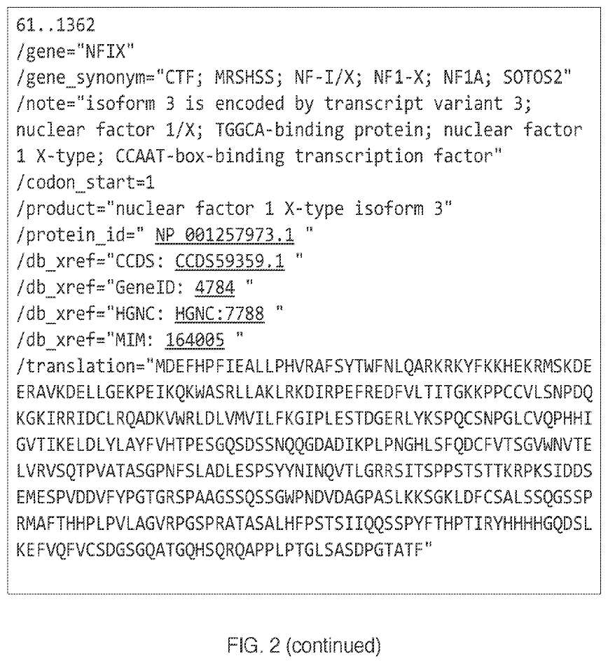

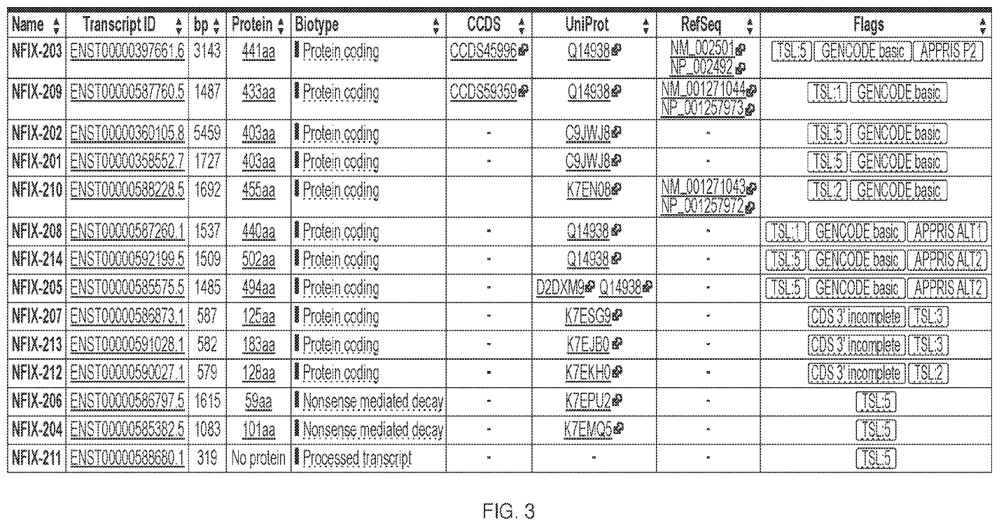

### DETAILED DESCRIPTION

The following definitions apply to the compositions, methods, and uses described herein (i.e., throughout the specification) unless the context clearly indicates otherwise.

The term “about,” when used in reference to a value, signifies any value or range of values that is plus-or-minus 10% of the stated value (e.g., within plus-or-minus 1%, 2%, 3%, 4%, 5%, 6%, 7%, 8%, 9% or 10% of the stated value). For example, a dose of about 10 mg means any dose as low as 10% less than 10 mg (9 mg), any dose as high as 10% more than 10 mg (11 mg), and any dose or dosage range therebetween (e.g., 9-11 mg; 9.1-10.9 mg; 9.2-10.8 mg; and so on). Where a stated value cannot be exceeded (e.g., 100%), “about” signifies any value or range of values that is up to and including 10% less than the stated value (e.g., a purity of about 100% means 90%-100% pure (e.g., 95%-100% pure, 96%-100% pure, 97%-100% pure etc . . . )). In the event an instrument or technique measuring a value has a margin of error greater than 10%, a given value will be about the same as a stated value when they are both within the margin of error for that instrument or technique.

The term “allogeneic” refers to any material (e.g., a biological cell, as described herein) obtained from or derived from a different individual of the same species as an individual to whom the material is introduced (e.g., allogeneic cells would be those obtained from a first human and administered to a second human). Two individuals are allogeneic to one another when the genes at one or more loci in the first individual are not identical to the genes at the same loci in the second individual.

The term “autologous” describes any material (e.g., a biological cell, as described herein) obtained from or derived from the same individual to whom it is later to be re-introduced.

The term “encoding” refers to the inherent property of specific sequences of nucleotides in a polynucleotide (e.g., a gene, a cDNA, or an mRNA) to serve as templates for synthesis of other polymers and macromolecules having either a defined sequence of nucleotides (e.g., rRNA, tRNA and mRNA) or a defined sequence of amino acids and the biological properties resulting therefrom. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein the term “exosome” refers to a cell-derived small (between 20-300 nm in diameter, more preferably 40-200 nm in diameter) vesicle comprising a membrane that encloses an internal space, and which is generated from the cell by direct plasma membrane budding or by fusion of the late endosome with the plasma membrane. The exosome comprises lipid or fatty acid and polypeptide and optionally comprises a payload (e.g., a therapeutic agent), a receiver (e.g., a targeting moiety), a polynucleotide (e.g., a nucleic acid, RNA, or DNA, such as any of the engineered nucleic acids described herein), a sugar (e.g., a simple sugar, polysaccharide, or glycan) or other molecules. The exosome can be derived from a producer cell, and isolated from the producer cell based on its size, density, biochemical parameters, or a combination thereof. An exosome is a species of extracellular vesicle. Generally, exosome production/biogenesis does not result in the destruction of the producer cell. Exosomes and preparation of exosomes are described in further detail in WO 2016/201323, corresponding to U.S. Application Publication No. 2018-0177727, which is hereby incorporated by reference in its entirety.

Unless otherwise specified, a “nucleotide sequence encoding an amino acid sequence” includes all nucleotide sequences that are degenerate versions of each other and that encode the same amino acid sequence. A “nucleotide sequence encoding an amino acid sequence” encompasses DNA sequences that include introns as well as RNA sequences that include or exclude introns (as is the case, for example, with precursor mRNAs and mature mRNAs, respectively).

The term “expression” refers to the transcription and/or translation of a particular nucleotide sequence driven by a promoter. The term “transfer vector” refers to a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses (e.g., non-naturally occurring viruses). Thus, the term “transfer vector” includes an autonomously replicating plasmid or a virus. The term should also be construed to further include non-plasmid and non-viral compounds that facilitate transfer of nucleic acids into cells, such as, for example, nanoparticles (e.g., comprising a polylysine compound), liposomes, and the like. Examples of viral transfer vectors include, but are not limited to, adenoviral vectors, adeno-associated virus vectors, retroviral vectors, lentiviral vectors, and the like. The term “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, including cosmids, plasmids (e.g., naked or contained in liposomes) and viruses (e.g., lentiviruses, retroviruses, adenoviruses, and adeno-associated viruses) that incorporate the recombinant polynucleotide.

The term “hematopoietic stem cell” or “HSC” refers to multipotent stem cells that give rise to all of the blood cell types of an organism, including myeloid (e.g., monocytes and macrophages, neutrophils, basophils, eosinophils, erythrocytes, megakaryocytes/platelets, dendritic cells), and lymphoid lineages (e.g., T-cells, B-cells, NK-cells), and others known in the art (see U.S. Pat. Nos. 5,635,387; 5,460,964; 5,677,136; 5,750,397; 5,759,793; 5,681,599; and 5,716,827). As would be understood in the art, a stem cell has the capacity for self-renewal; as a stem cell proliferates, it produces various differentiated cell types as well as more of the same stem cell. Unlike stem cells, progenitor cells do not have self-renewal potential.

A “hemoglobinopathy” is a hereditary condition involving an abnormality in the structure of hemoglobin or in the amount of hemoglobin produced or present in an affected cell.

The term “homologous” or “identity” refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous or identical at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

The terms “in vitro” and “ex vivo” refer to an event that takes places outside of an organism (e.g., a patient's body). In vitro assays encompass cell-based assays as well as cell-free or biochemical assay in which no intact cells are utilized. An “ex vivo” event can involve treating or performing a procedure on a cell, tissue, or organ that has been removed from a patient's body. As and when appropriate, the cell, tissue, or organ may be returned to a subject's body by a suitable medical procedure.

The term “in vivo” refers to an event that takes place in an organism (e.g., a patient's body).

A promoter is “inducible” when it is operably linked with a polynucleotide that encodes or specifies a gene product and causes the gene product to be produced in a cell substantially only when an inducer corresponding to the promoter is present in the cell.

The term “isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell. We use the commonly accepted abbreviations for commonly occurring nucleic acid bases (“A” for adenosine, “C” for cytosine, “G” for guanosine, “T” for thymidine, and “U” for uridine).

The term “lentivirus” refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses that can be genetically modified and used as lentiviral vectors as described herein.

The term “lentiviral vector” refers to a vector derived from at least a portion of a lentivirus genome, including especially a self-inactivating lentiviral vector as provided in Milone et al. (Mol. Ther. 17(8):1453-1464, 2009). Other examples of lentivirus vectors that may be used in the clinic, include but are not limited to, e.g., the LENTIVECTOR® gene delivery technology from Oxford BioMedica, the LENTIMAX™ vector system from Lentigen and the like. Nonclinical types of lentiviral vectors are also available and would be known to one skilled in the art.

As used herein, the term “nanovesicle” (also referred to as a “microvesicle”) refers to a cell-derived small (between 20-250 nm in diameter, more preferably 30-150 nm in diameter) vesicle comprising a membrane that encloses an internal space, and which is generated from the cell by direct or indirect manipulation such that the nanovesicle would not be produced by the producer cell without manipulation. In general, a nanovesicle is a sub-species of an extracellular vesicle. Appropriate manipulations of the producer cell include but are not limited to serial extrusion, treatment with alkaline solutions, sonication, or combinations thereof. The production of nanovesicles may, in some instances, result in the destruction of the producer cell. Preferably, populations of nanovesicles are substantially free of vesicles that are derived from producer cells by way of direct budding from the plasma membrane or fusion of the late endosome with the plasma membrane. The nanovesicle comprises lipid or fatty acid and polypeptide, and optionally comprises a payload (e.g., a therapeutic agent), a receiver (e.g., a targeting moiety), a polynucleotide (e.g., a nucleic acid, RNA, or DNA, such as any of the engineered nucleic acids described herein), a sugar (e.g., a simple sugar, polysaccharide, or glycan) or other molecules. The nanovesicle, once it is derived from a producer cell according to such manipulation, may be isolated from the producer cell based on its size, density, biochemical parameters, or a combination thereof.

The terms “nucleic acid” and “polynucleotide” refer to deoxyribonucleic acid (DNA) or ribonucleic acid (RNA), or to polymers containing a combination of DNA or RNA in either single- or double-stranded form. We may use the term “nucleic acid” to refer to any segment of a polynucleotide, whether or not naturally occurring, and to a “nucleic acid construct” as any artificially constructed (i.e., non-naturally occurring) moiety containing a nucleic acid, alone or associated with additional components (e.g., an enzyme or vector such as a plasmid or viral vector). For example, a nucleic acid construct including a guide RNA can be associated with a protein, such as Cas9. Nucleic acid constructs within a cell of the invention or used in a method described herein may contain only naturally occurring nucleotides or may include analogues or derivatives of natural nucleotides.

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, or a combination thereof.

The term “parenteral” refers to administration of a composition described herein by a route other than administration by mouth or to the alimentary canal (e.g., subcutaneous, intravenous, or intramuscular administrations are parenteral administrations).

The term “patient” refers to any organism, including a human being, to which a composition described herein is administered in accordance with the present invention, e.g., for treating a hemoglobinopathy.

The term “pharmaceutically acceptable” as applied to compositions (e.g., pharmaceutical compositions and component parts thereof (e.g., nucleic acid constructs that inhibit the expression of NFIX, cells comprising such constructs, carriers, diluents and reagents (e.g., media suitable for a genetically modified cell, as described herein))) represent that the composition can be administered to a patient without toxic effect. Each component must also be “pharmaceutically acceptable” in the sense of being compatible with the other ingredients of the formulation. Typically, such compositions are prepared as injectable either as liquid solutions or suspensions, however, solid forms suitable for solution, or suspensions, in liquid prior to use can also be prepared. The preparation can also be emulsified or presented as a liposome composition. The active ingredient can be mixed with excipients that are pharmaceutically acceptable and compatible with the active ingredient and in amounts suitable for use in the therapeutic methods described herein. Suitable excipients are, for example, water, saline, dextrose, glycerol, ethanol or the like and combinations thereof. In addition, if desired, the composition can contain minor amounts of auxiliary substances such as wetting or emulsifying agents, pH buffering agents and other such agents that enhance the effectiveness of the active ingredient (i.e., a nucleic acid construct that inhibits the expression of NFIX). The therapeutic composition of the present invention can include pharmaceutically acceptable salts of one or more of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic, and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active agent used in the invention that will be effective in the treatment of a particular disorder or condition (i.e., a hemaglobinopathy) will depend on the nature of the disorder or condition and can be determined by one of ordinary skill in the art.

The term “promoter” refers to a nucleic acid (e.g., DNA) sequence that is recognized by the synthetic machinery of a biological cell and which is required to initiate transcription of a polynucleotide sequence (e.g., a naturally occurring gene or a genetically engineered sequence). A promoter may reside naturally within a genome or may be introduced into a cell by human intervention. In either event, the promoter can be “tissue-specific” by virtue of demonstrating activity in only certain types of cells (e.g., HSCs, HPCs, or erythrocytes). The compositions of the invention (e.g., genetically modified cells and compositions containing them (e.g., pharmaceutical compositions)) can include a promoter, optionally a tissue-specific promoter, operably linked to a nucleic acid that inhibits the expression of NFIX, and any such compositions find utility in the methods of treating hemoglobinopathies as described herein.

The term “promoter/regulatory sequence” refers to a nucleic acid sequence that is required for expression of a gene product operably linked to the promoter/regulatory sequence. In some instances, this sequence may be the core promoter sequence and, in other instances, this sequence may also include an enhancer sequence and/or other regulatory elements that are required for efficient expression of the gene product. The term “constitutive” promoter refers to a nucleotide sequence that, when operably linked with a polynucleotide that encodes or specifies a gene product, causes the gene product to be produced in a cell under most or all physiological conditions of the cell.

We use the terms “substantially purified” and “isolated” to refer to biological material (e.g., a cell, such as a HSC, HPC, or erythrocyte) that has been removed from the naturally occurring setting (e.g., a human body) in which it normally resides. In some instances, a substantially purified or isolated cell, or a population thereof, is essentially free of other cell types (i.e., a population of cells used as described herein may be, but is not necessarily, a homogenous population of cells). In some instances, the cells are cultured or otherwise maintained in vitro prior to administration to a patient (and during which time they can be genetically modified as described herein).

We use the term “syngeneic” to refer to any material (e.g., a biological cell, as described herein) obtained from or derived from one individual who is genetically identical to a second individual to whom the material is administered.

“Xenogeneic,” as used herein, refers to any material (e.g., a biological cell, as described herein) obtained from or derived from an individual of a different species from the individual to whom it is introduced.

Throughout this disclosure, various aspects of the invention may be described as within a specified range. It is to be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. As another example, a range such as 95-99% identity, includes something with 95%, 96%, 97%, 98% or 99% identity, and includes subranges such as 96-99%, 96-98%, 96-97%, 97-99%, 97-98% and 98-99% identity. This applies regardless of the breadth of the range.

Inhibiting NFIX with RNAi knockdown technology: In some embodiments, NFIX mRNA expression is inhibited using RNA interference (RNAi) knockdown technology (e.g., a short-interfering RNA (siRNA) system). In some embodiments, NFIX mRNA expression is decreased using RNAi using short-hairpin RNA (shRNA) molecules.

Synthetic siRNA molecules, including shRNA molecules, to inhibit the expression of NFIX can be obtained using a number of techniques known to one of ordinary skill in the art. For example, an siRNA molecule can be chemically synthesized or recombinantly produced using methods known in the art, such as using appropriately protected ribonucleoside phosphoramidites and a conventional DNA/RNA synthesizer (see, e.g., Elbashir et al., Nature 411:494-498, 2001; Elbashir et al., Genes Dev. 15:188-200, 2001; Harborth et al., J. Cell Science 114:4557-4565, 2001; Masters et al., Proc. Natl. Acad. Sci., USA 98:8012-8017, 2001; and Tuschl et al., Genes & Development 13:3191-3197, 1999). Alternatively, several commercial RNA synthesis suppliers are available including, but not limited to, Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA), and Cruachem (Glasgow, UK). One can also consult or utilize the resources at web.stanford.edu/group/markkaylab/cgi-bin/; see Zhang et al., Nucleic Acids Res. pii:gku854, 2014) or siSPOTR (www.sispotr.icts.uiowa.edu/sispotr/index.html; see Boudreau et al., Nucleic Acids Res., 2012, PMID: 22941647). Double-stranded RNAs (dsRNAs) can be expressed as stem loop structures encoded by plasmid vectors, retroviruses and lentiviruses (Paddison et al., Genes Dev. 16:948-958, 2001; McManus et al., RNA 8:842-850, 2002; Paul et al. Nat. Biotechnol. 20:505-508, 2002; Miyagishi et al., Nat. Biotechnol. 20:497-500, 2002; Sui et al., Proc. Natl. Acad. Sci., USA 99:5515-5520, 2002; Brummelkamp et al., Cancer Cell 2:243, 2002; Lee et al., Nat. Biotechnol. 20:500-505, 2002; Yu et al., Proc. Natl. Acad. Sci., USA 99:6047-6052, 2002; Zeng et al., Mol. Cell. 9:1327-1333, 2002; Rubinson et al. Nat. Genet. 33:401-406, 2003; Stewart et al., RNA 9:493-501, 2003). These vectors generally have a polIII promoter upstream of the dsRNA and can express sense and antisense RNA strands separately and/or as hairpin structures. In case of doubt, any of these types of vectors, including a promoter (e.g., a polIII promoter) as described herein can be used in the methods of the invention and may be a part of a composition described herein (e.g., a genetically modified cell in which NFIX expression is inhibited or a kit). shRNA_{miR}, comprising optimized shRNAs embedded within a microRNA (miRNA), can be driven by the polII promoter upstream, as described in U.S. Pat. No. 9,822,355, incorporated herein by reference.

The region targeted by a nucleic acid construct that inhibits the expression of NFIX (e.g., an siRNA molecule) can be selected from a given target gene sequence, e.g., an NFIX coding sequence, beginning from about 25 to 50 nucleotides, from about 50 to 75 nucleotides, or from about 75 to 100 nucleotides downstream of the start codon. Target nucleic sequences may contain 5′ or 3′ UTRs (untranslated regions) and regions nearby the start codon. One method of designing a siRNA molecule of the present invention involves identifying the 23 nucleotide sequence motif and selecting hits with at least 25%, 30%, 35%, 40%, 45%, 50%, 55%, 60%, 65%, 70% or 75% G/C content. Alternatively, if no such sequence is found, the search may be extended using the motif NA(N21), where N can be any nucleotide. In this situation, the 3′ end of the sense siRNA may be converted to TT to allow for the generation of a symmetric duplex with respect to the sequence composition of the sense and antisense 3′ overhangs. The antisense siRNA molecule may then be synthesized as the complement to nucleotide positions 1 to 21 of the 23-nucleotide sequence motif. The use of symmetric 3′ TT overhangs may be advantageous to ensure that the small interfering ribonucleoprotein particles (siRNPs) are formed with approximately equal ratios of sense and antisense target RNA-cleaving siRNPs (Elbashir et al., 2001, supra and Elbashir et al., 2001, supra). Analysis of sequence databases, including but not limited to the NCBI, BLAST, Derwent, and GenSeq as well as commercially available oligosynthesis companies such as OLIGOENGINE®, may also be used to select siRNA sequences against EST libraries to ensure that only one gene is targeted.

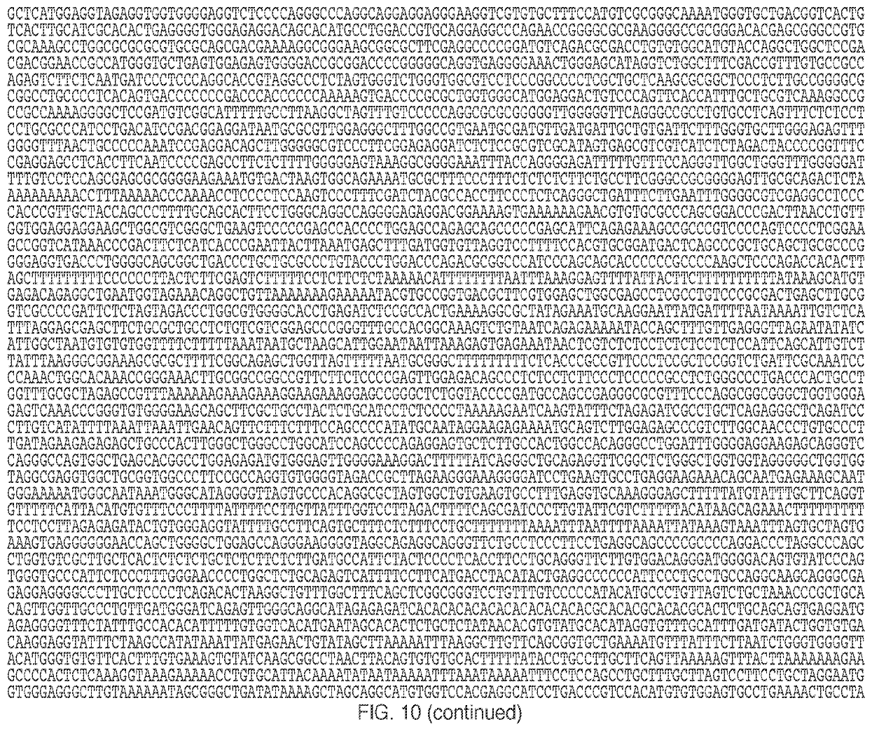

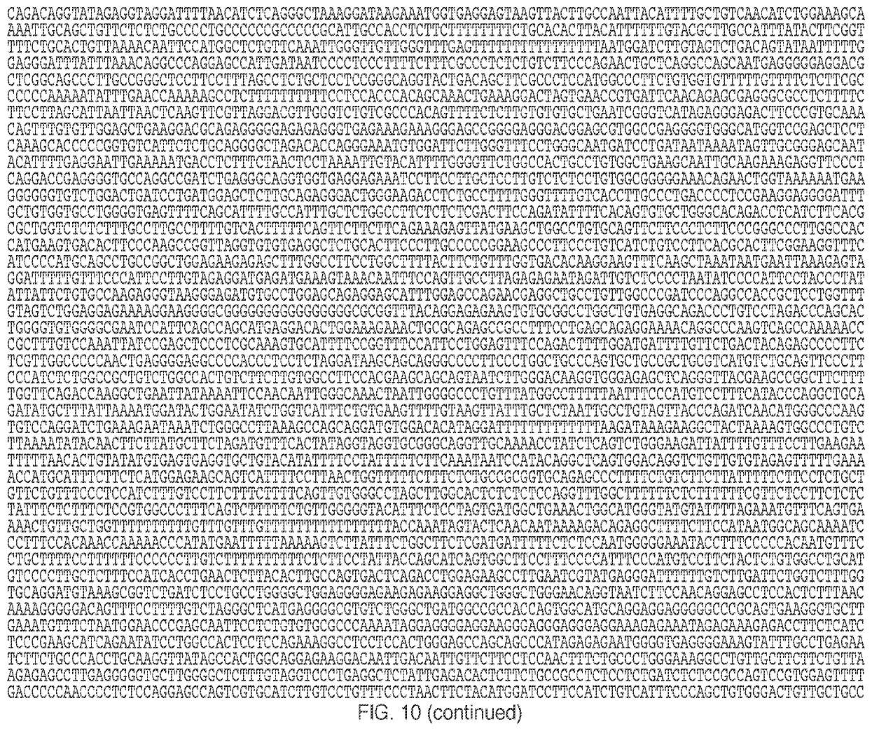

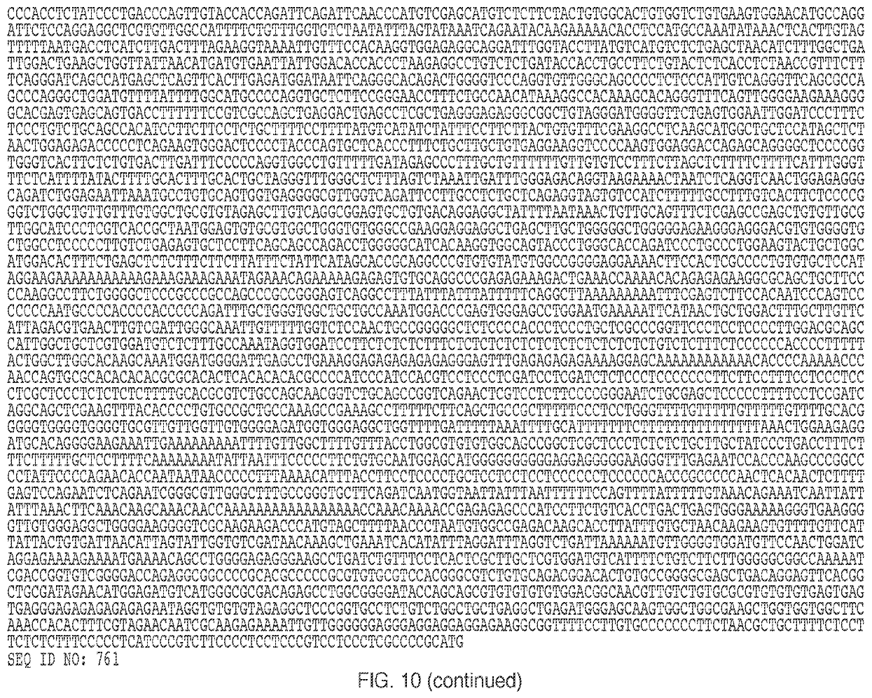

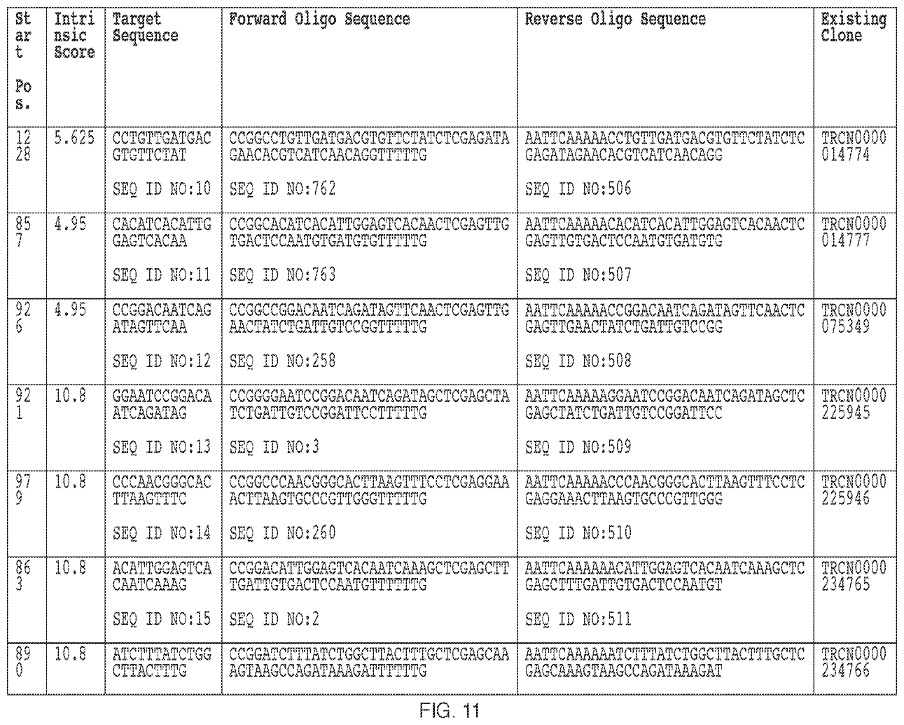

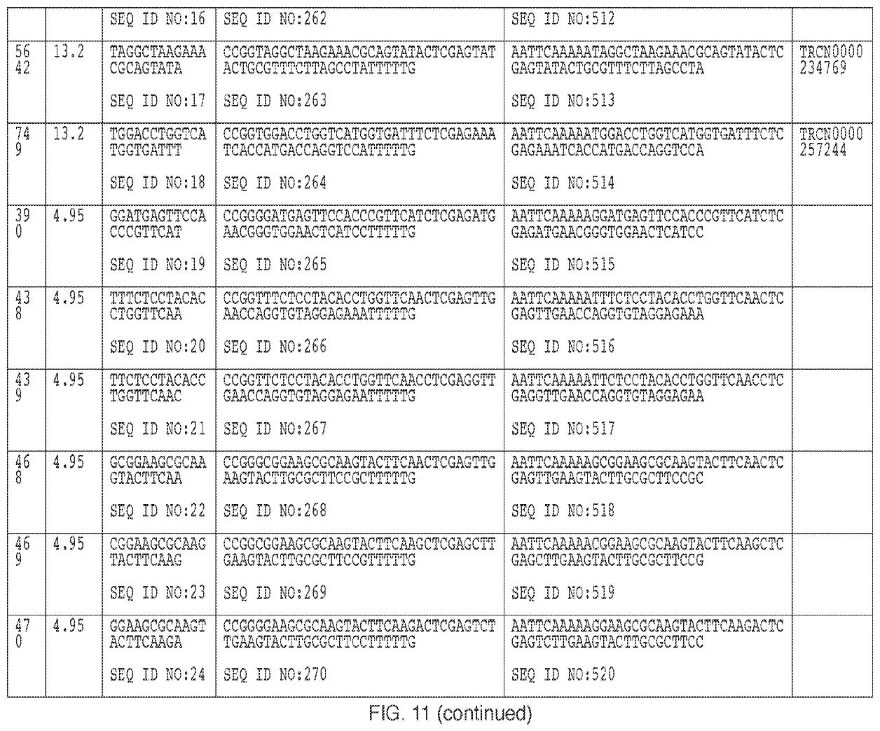

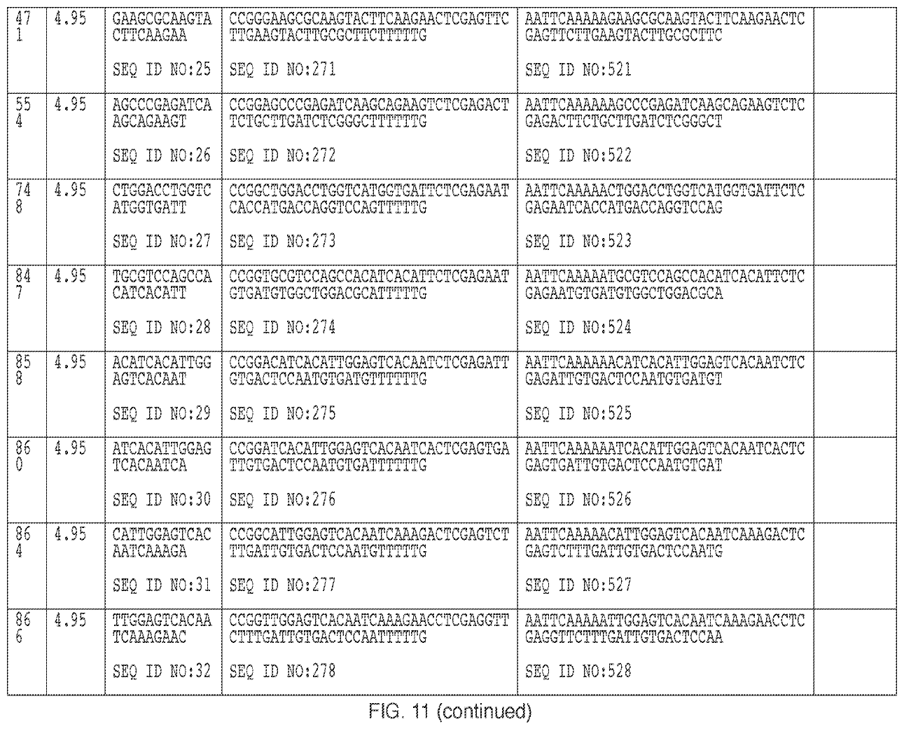

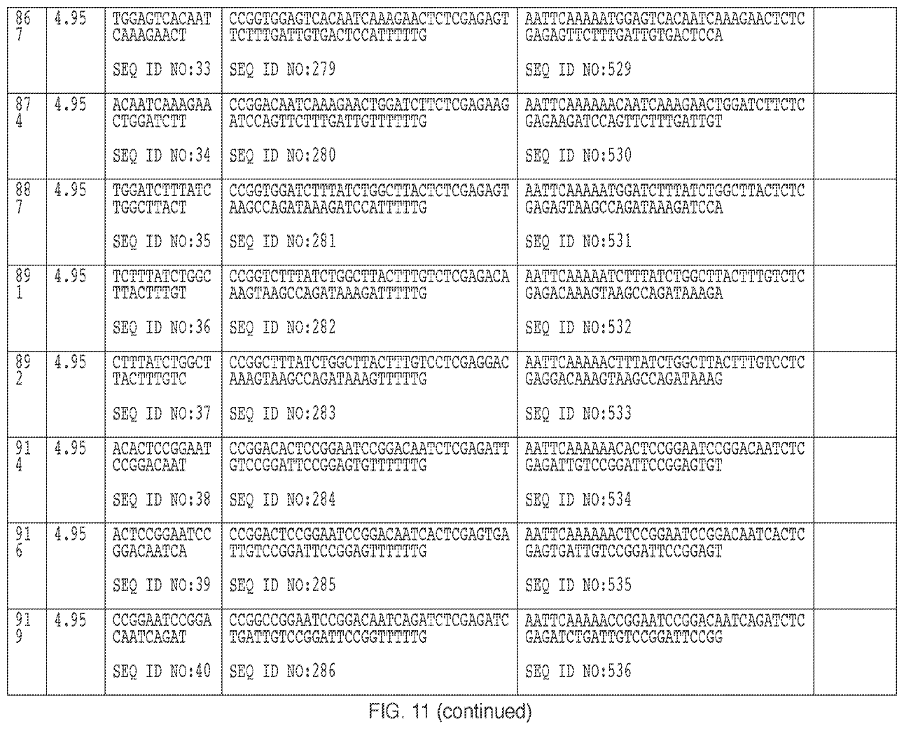

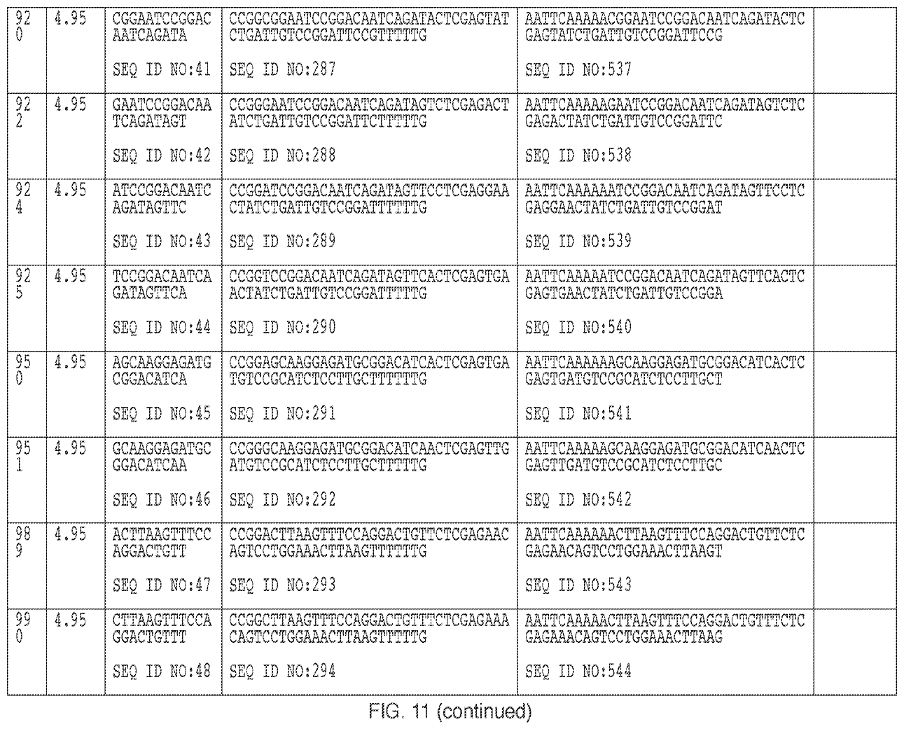

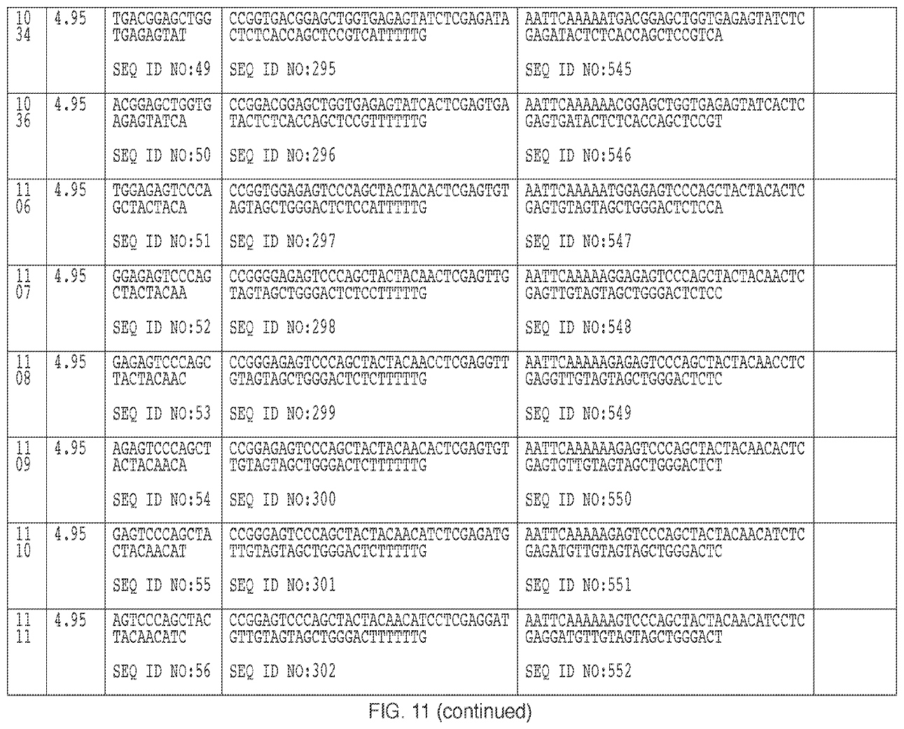

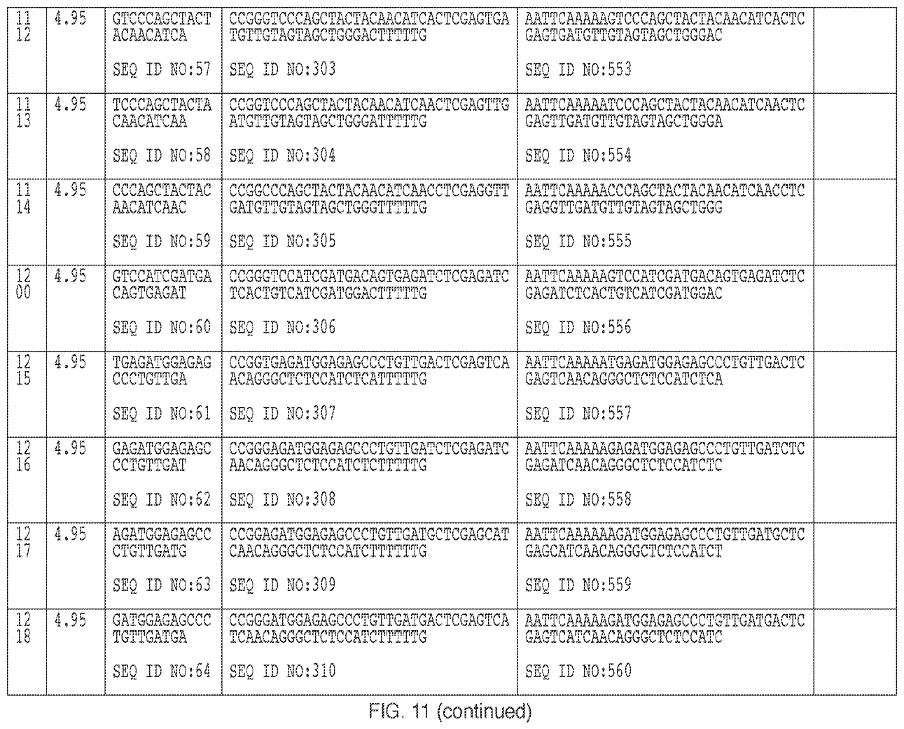

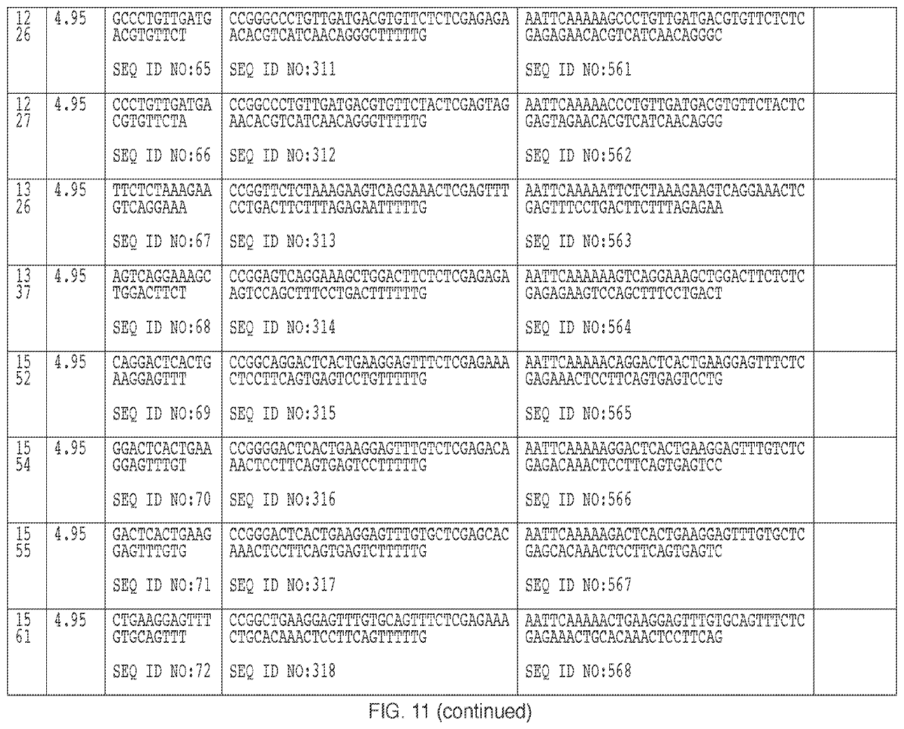

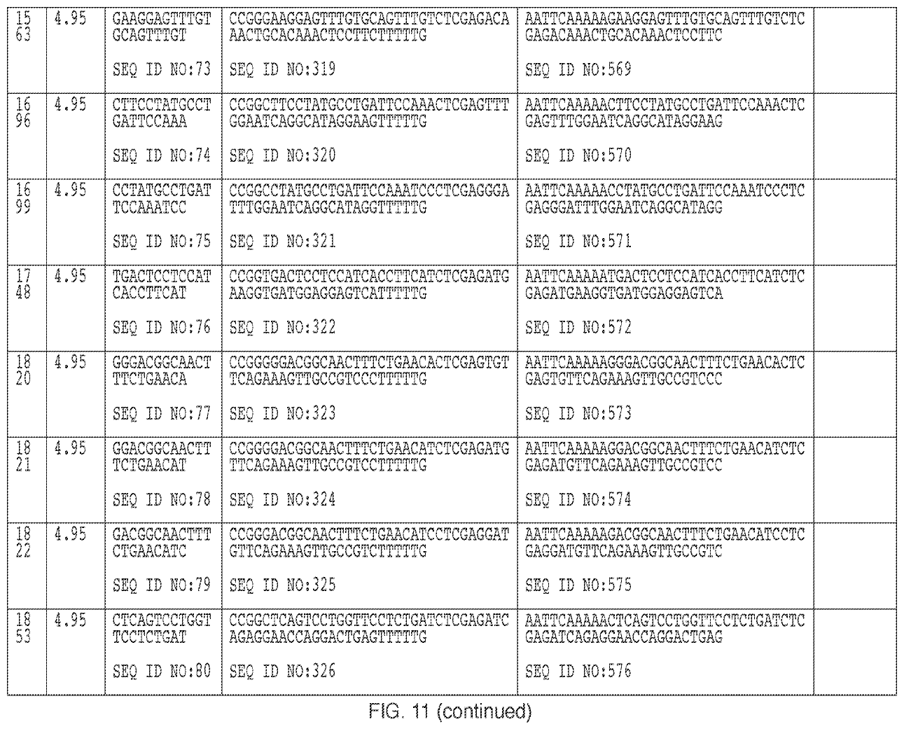

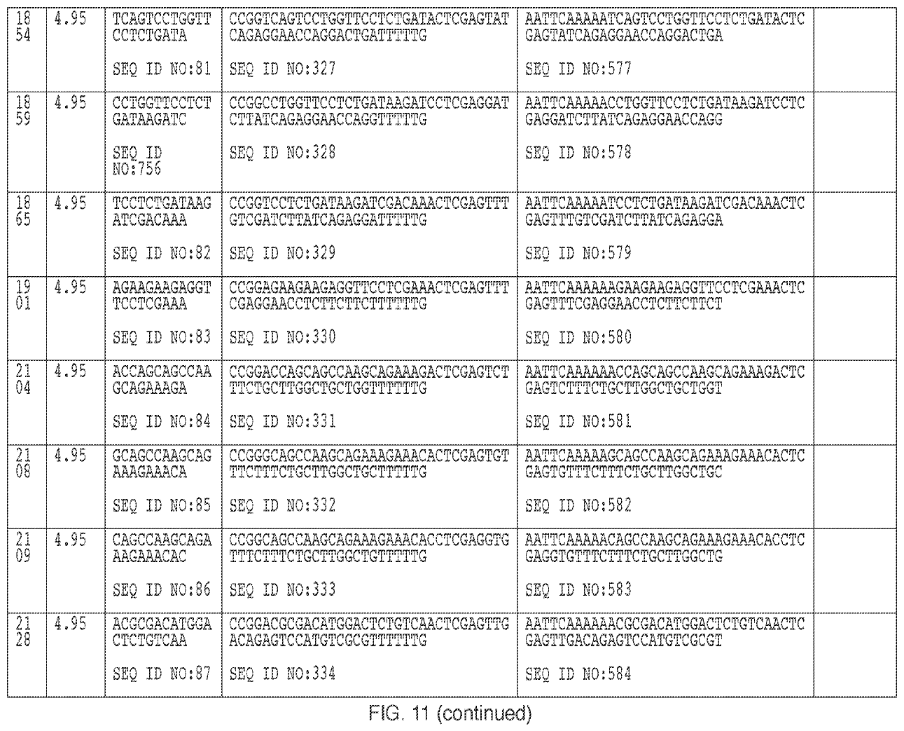

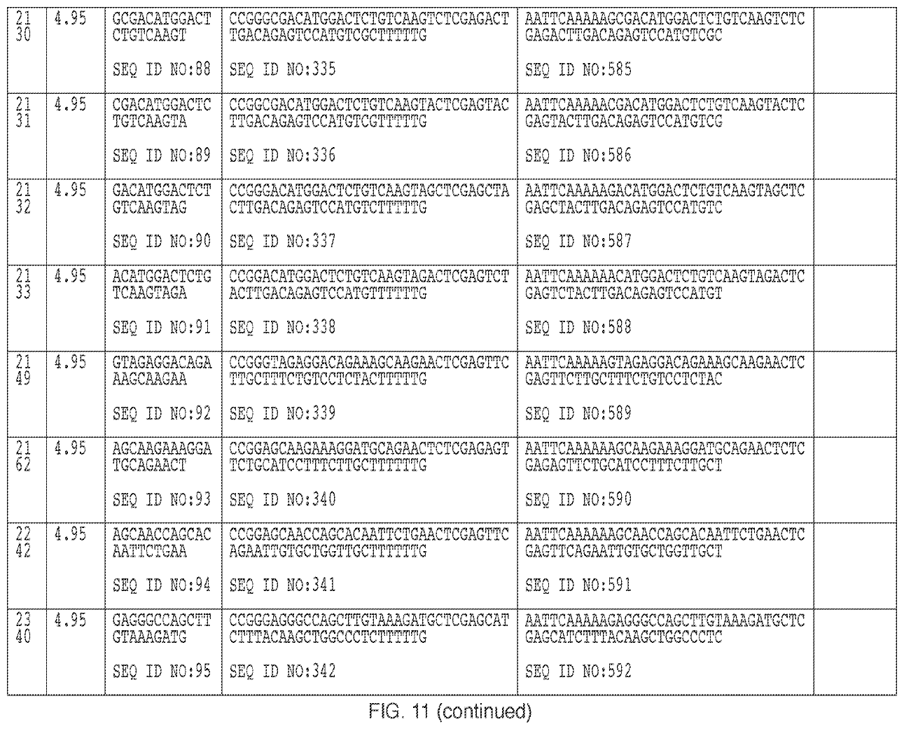

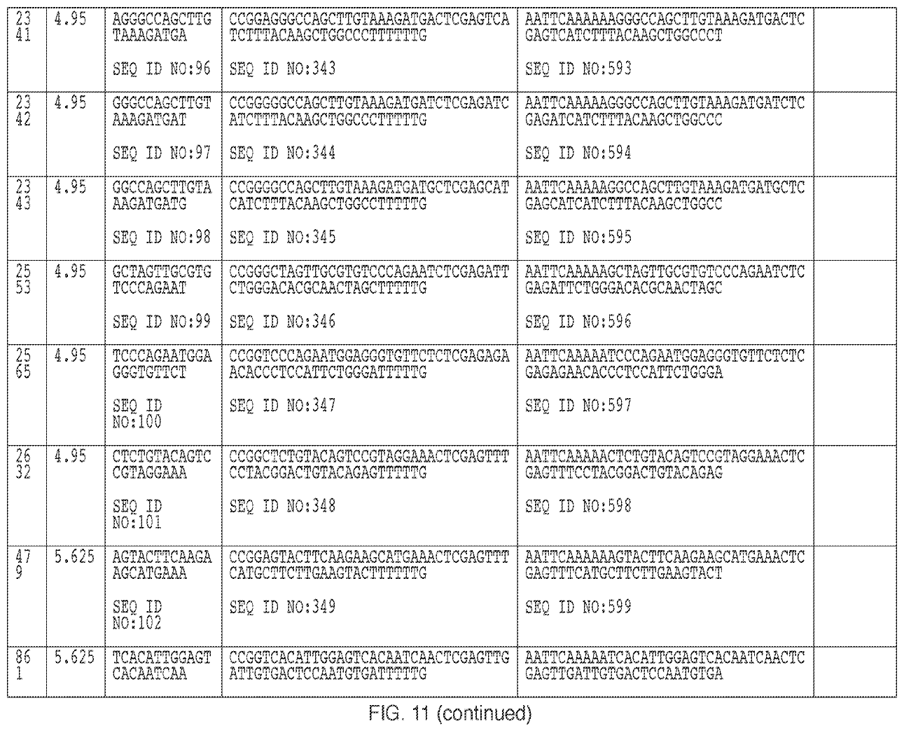

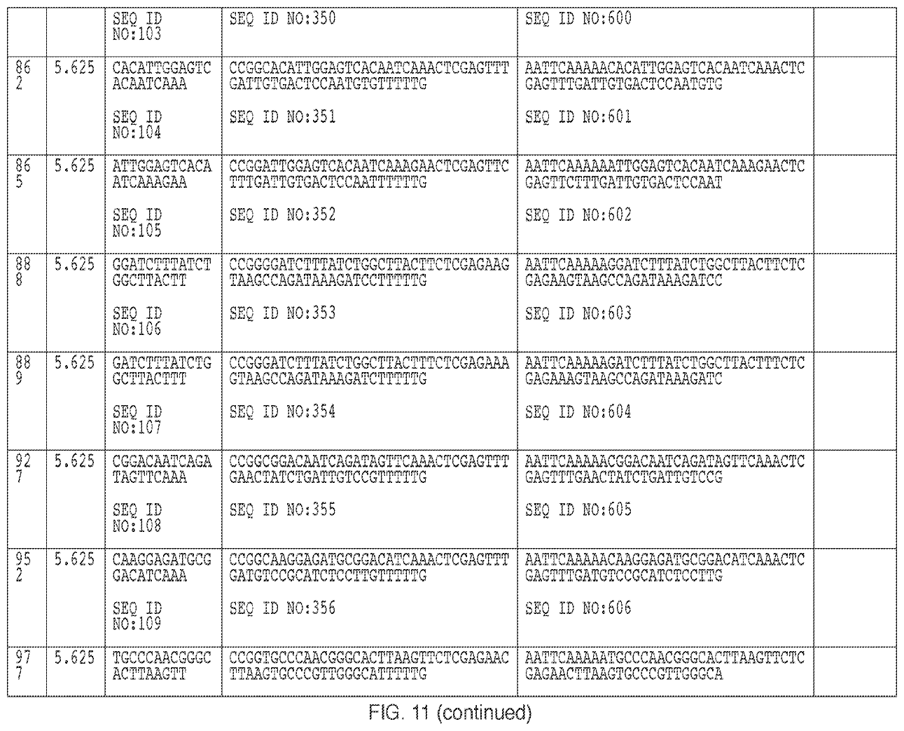

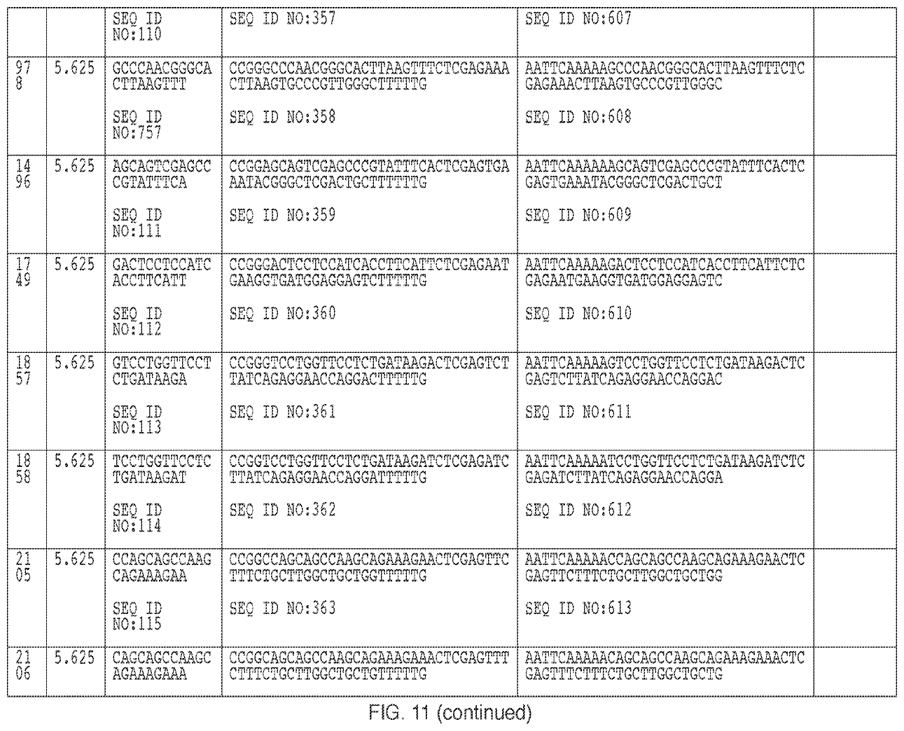

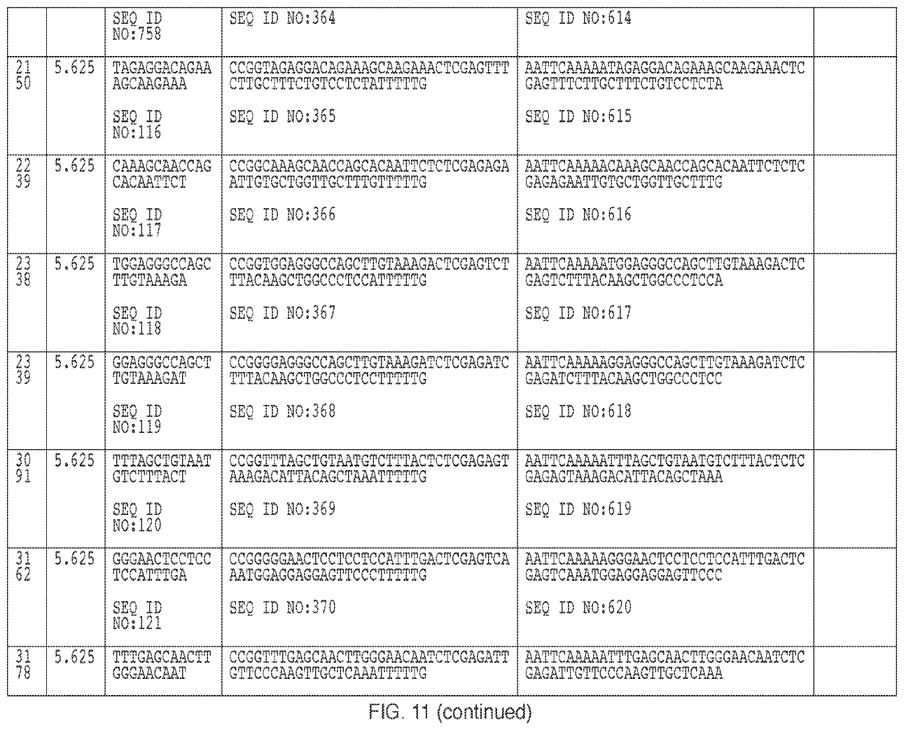

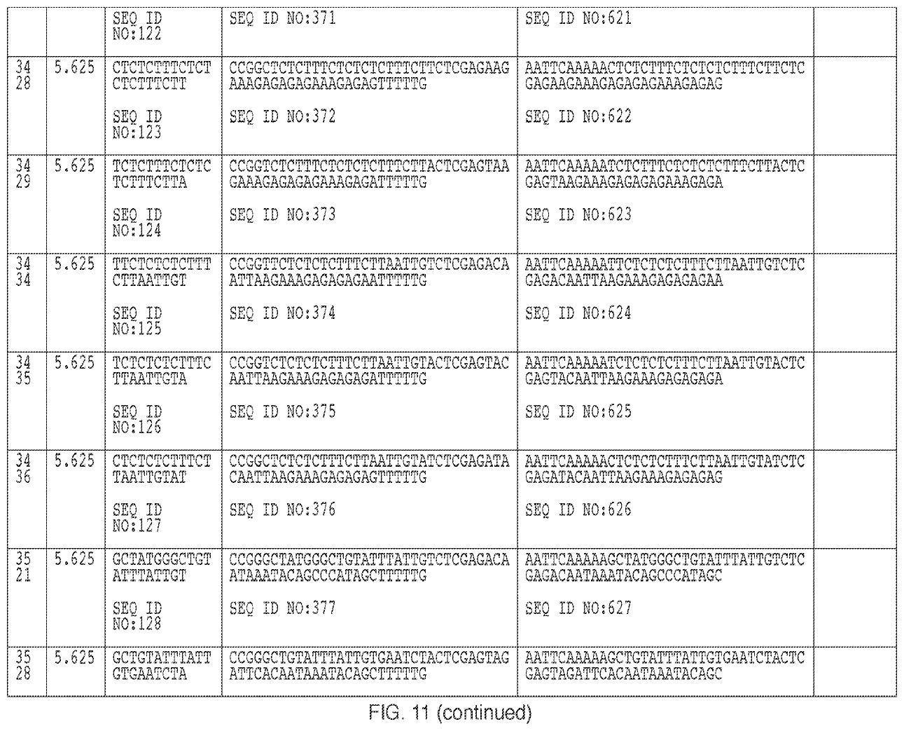

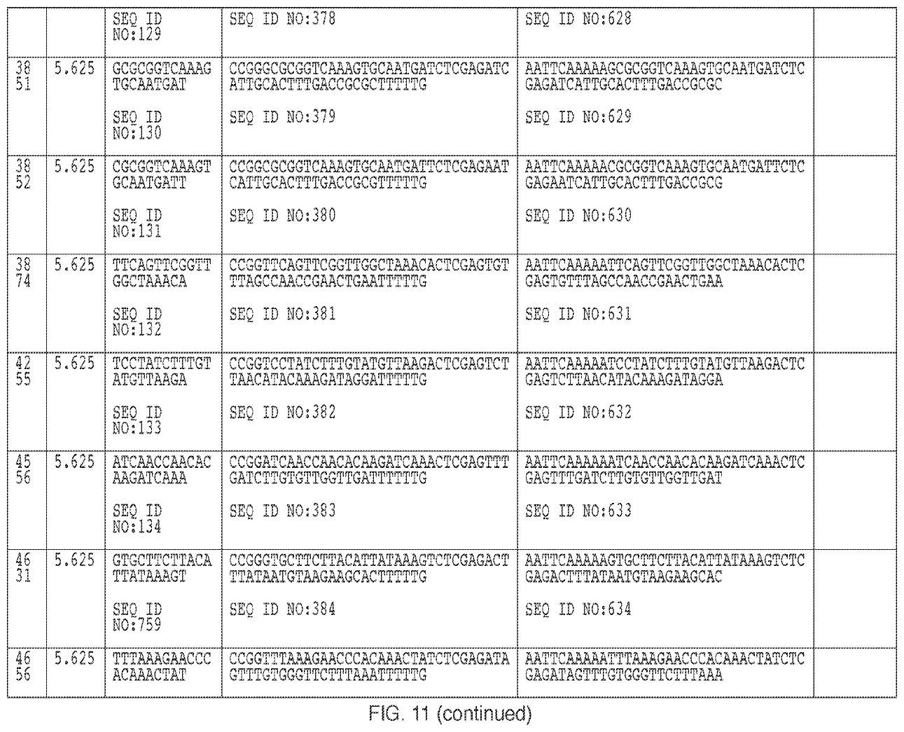

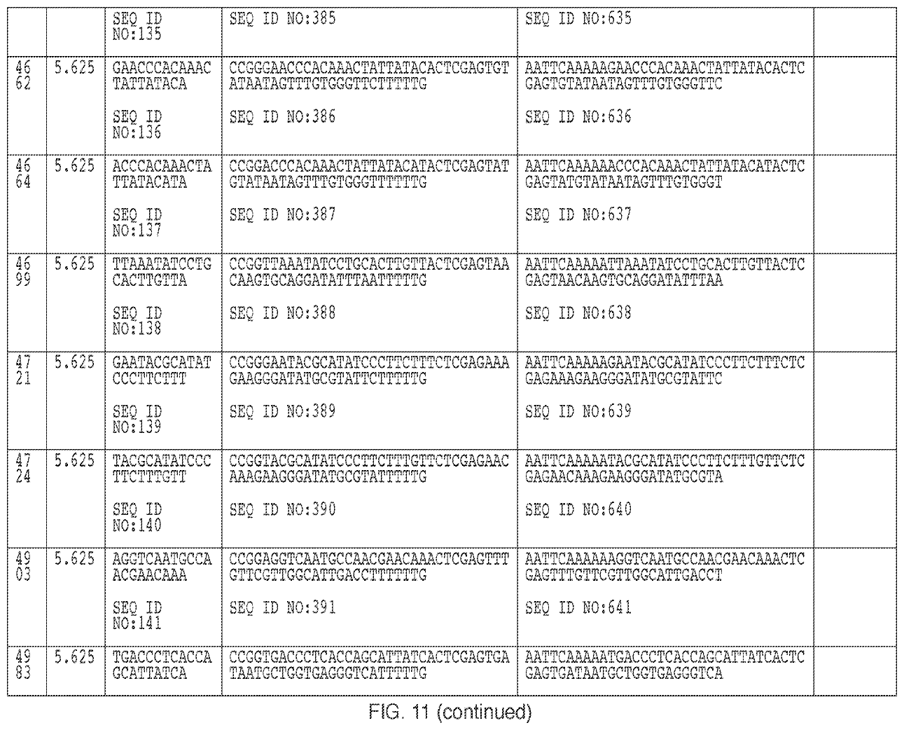

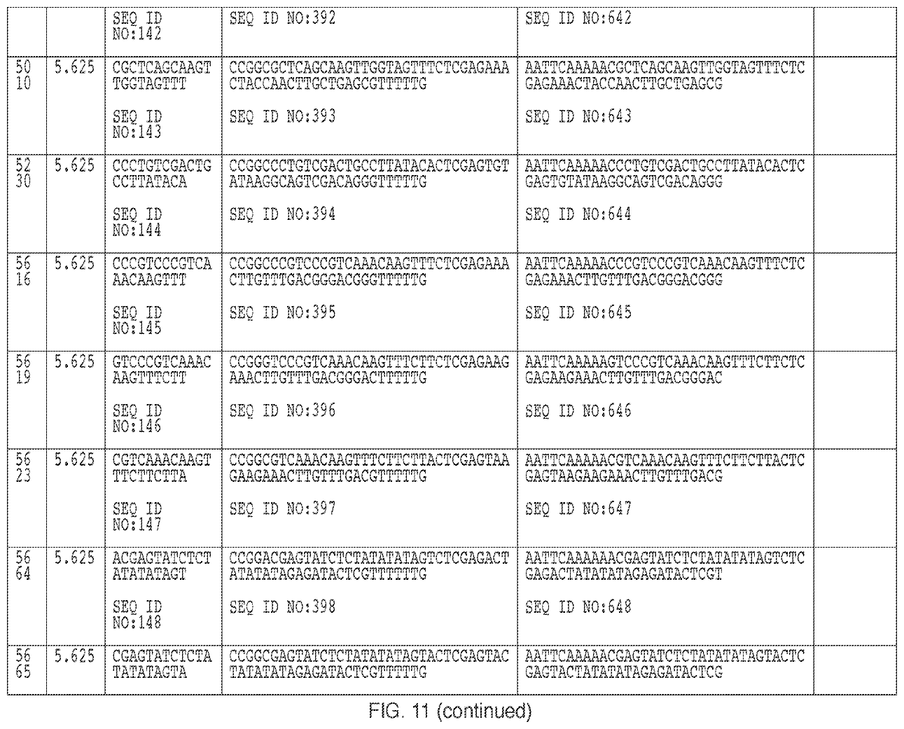

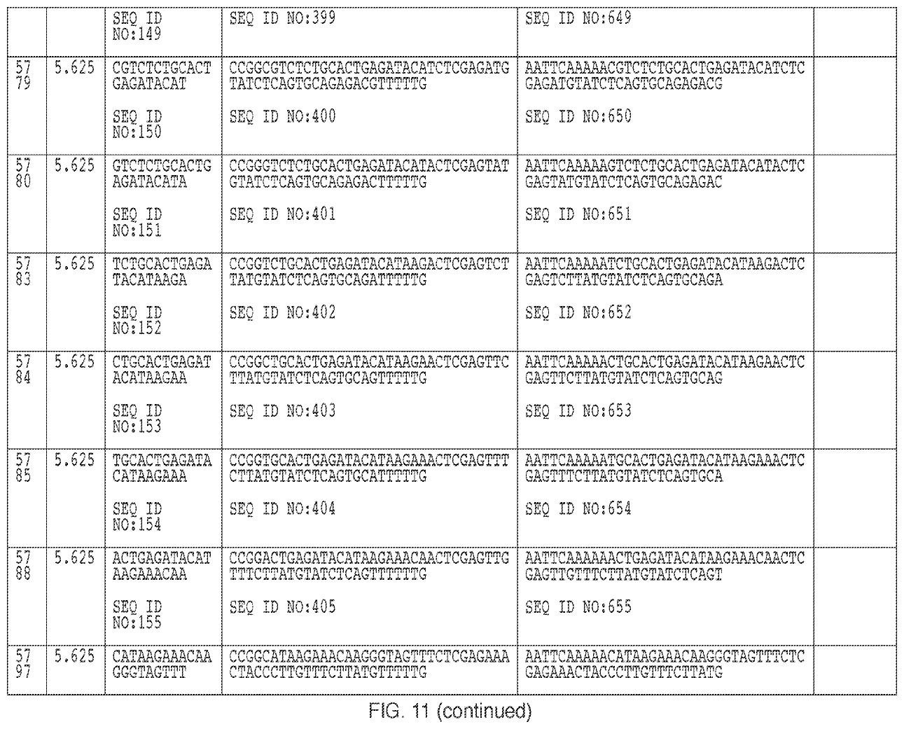

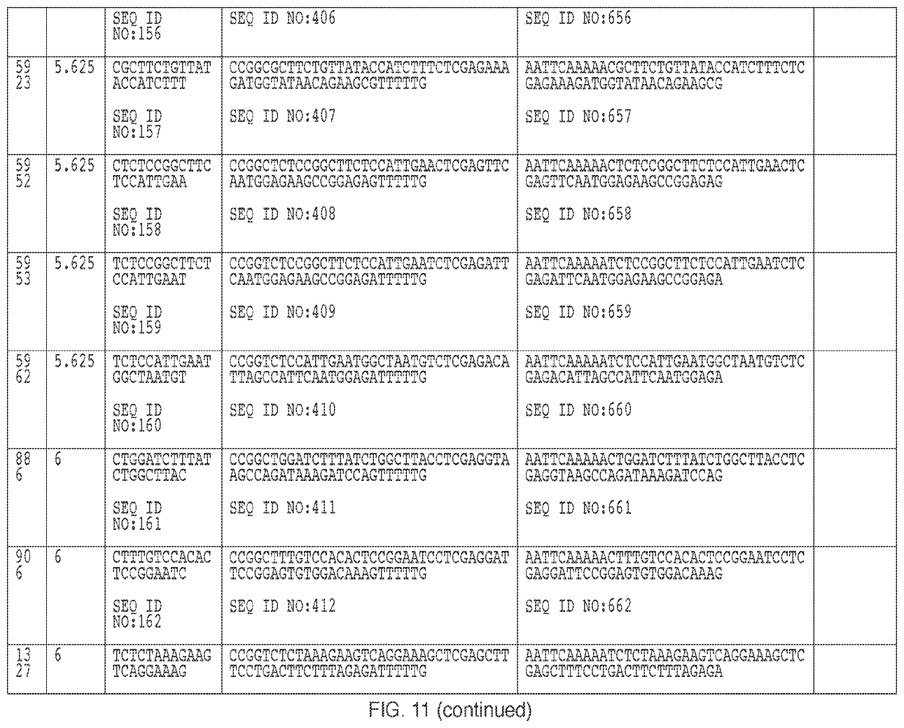

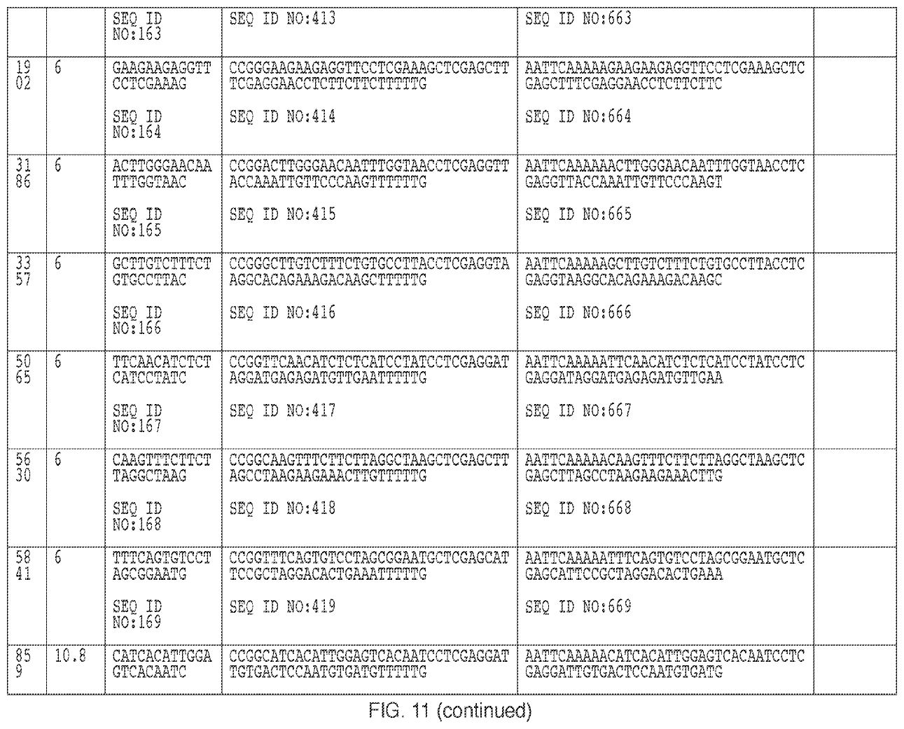

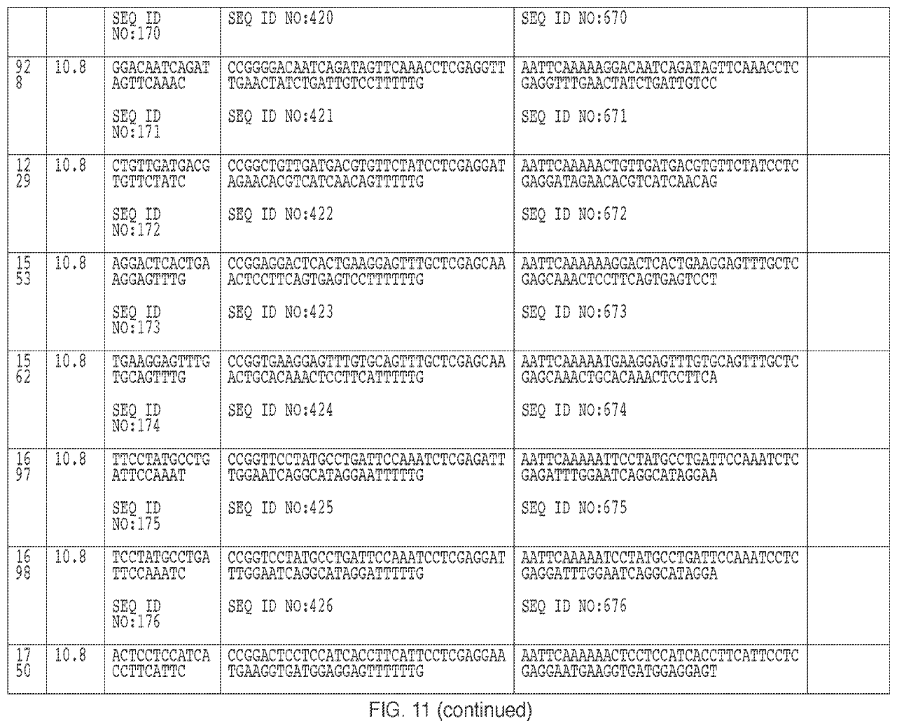

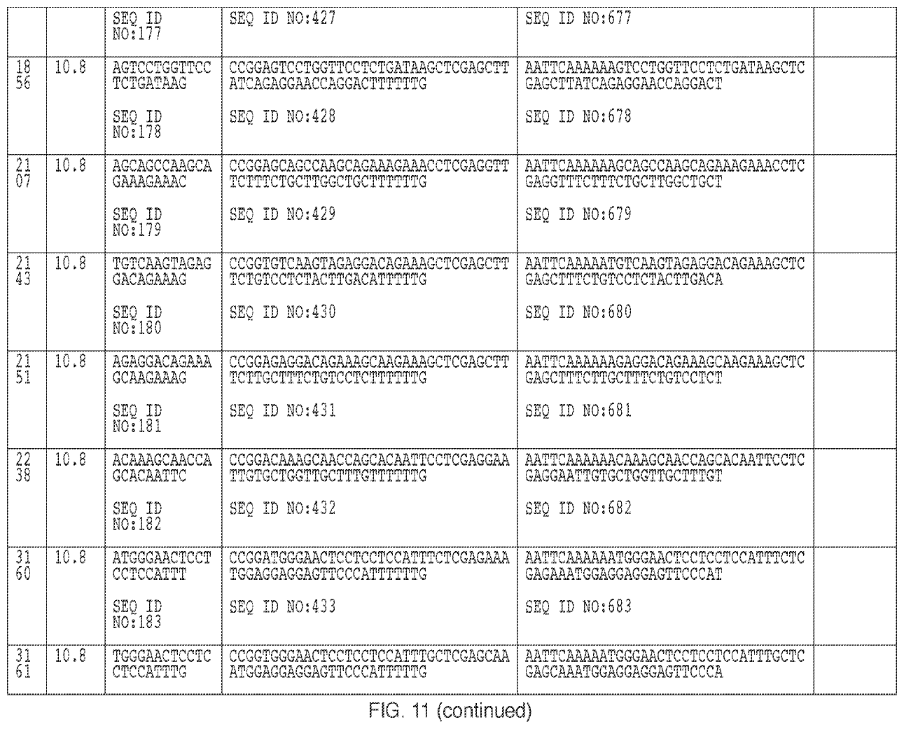

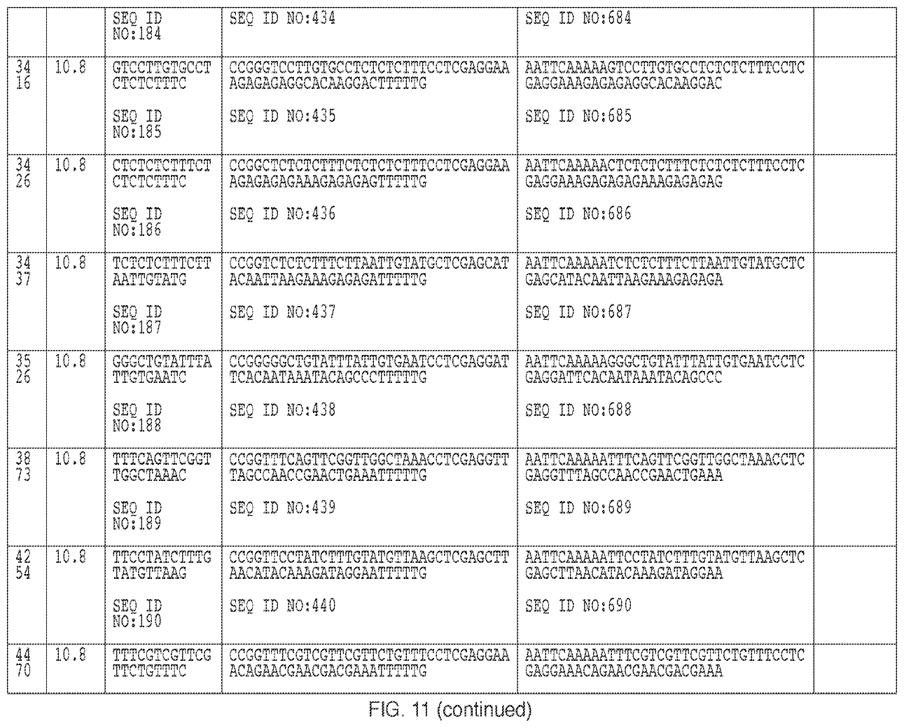

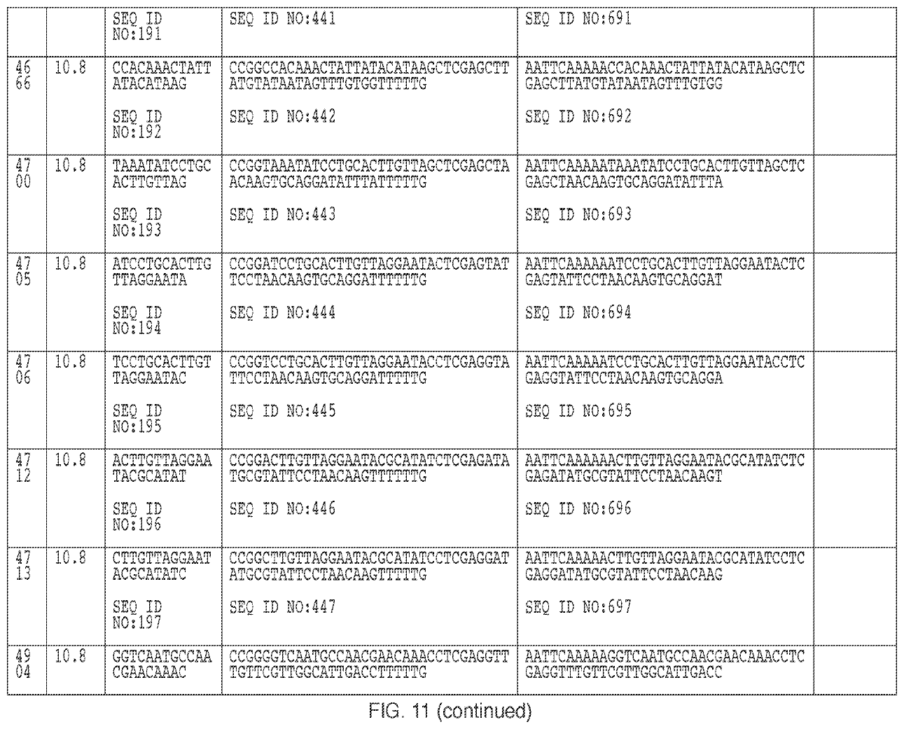

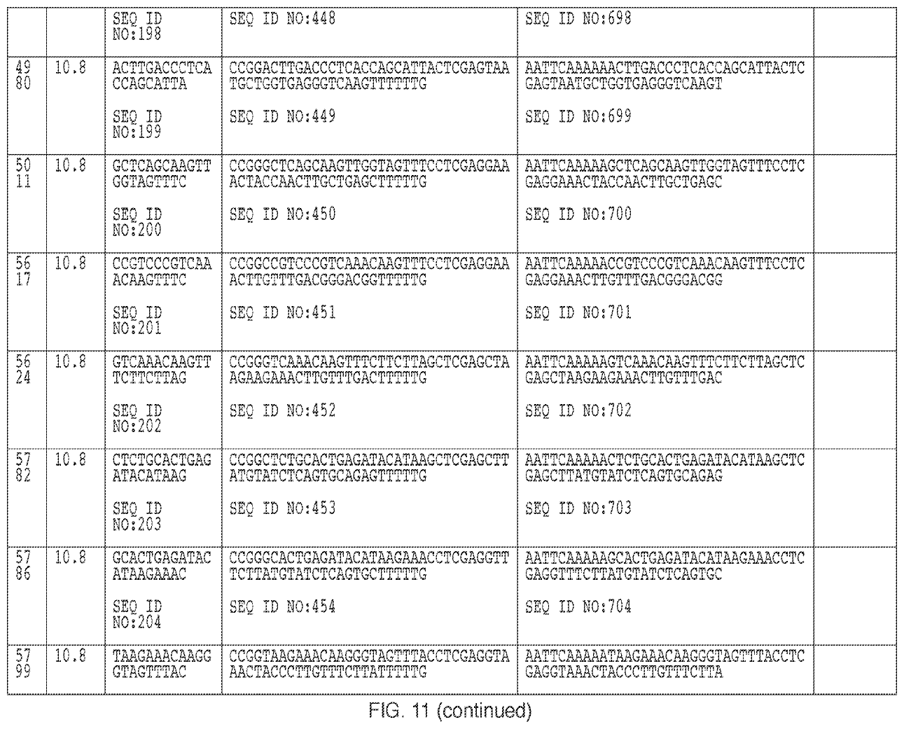

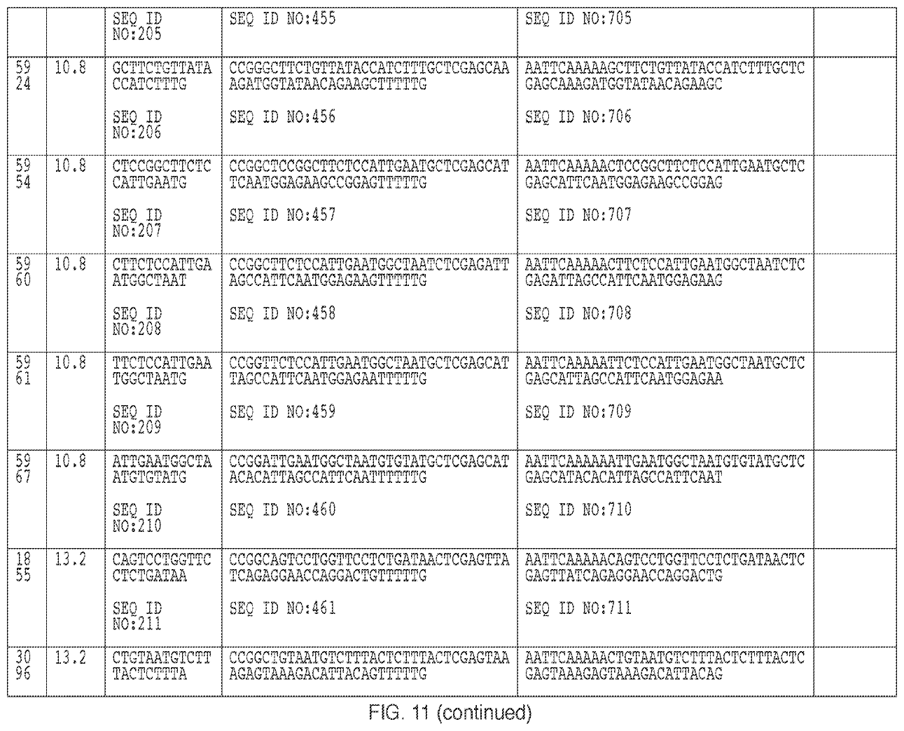

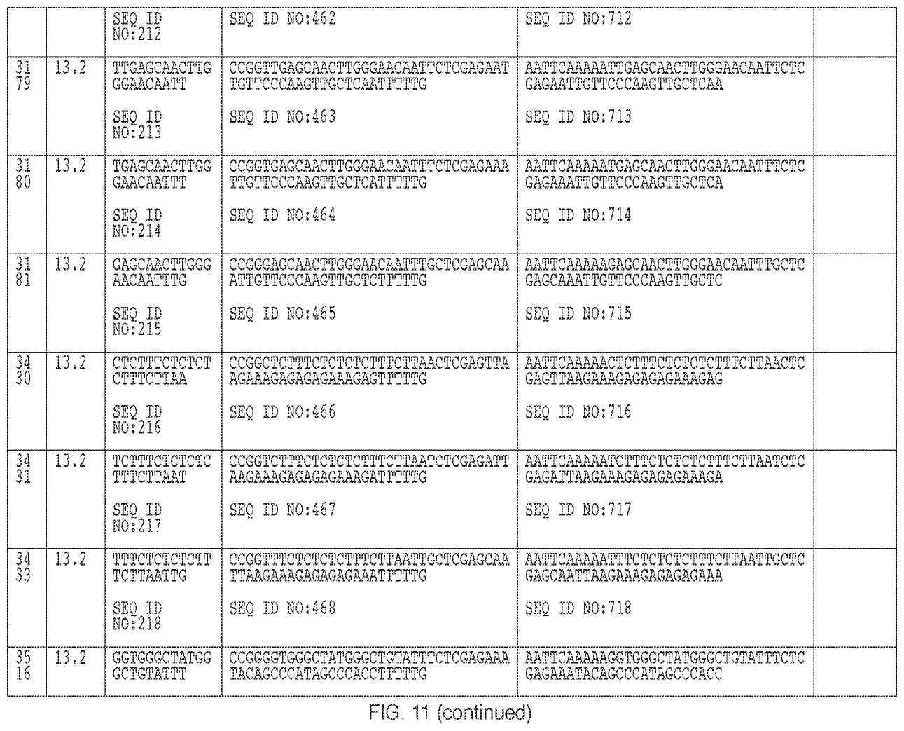

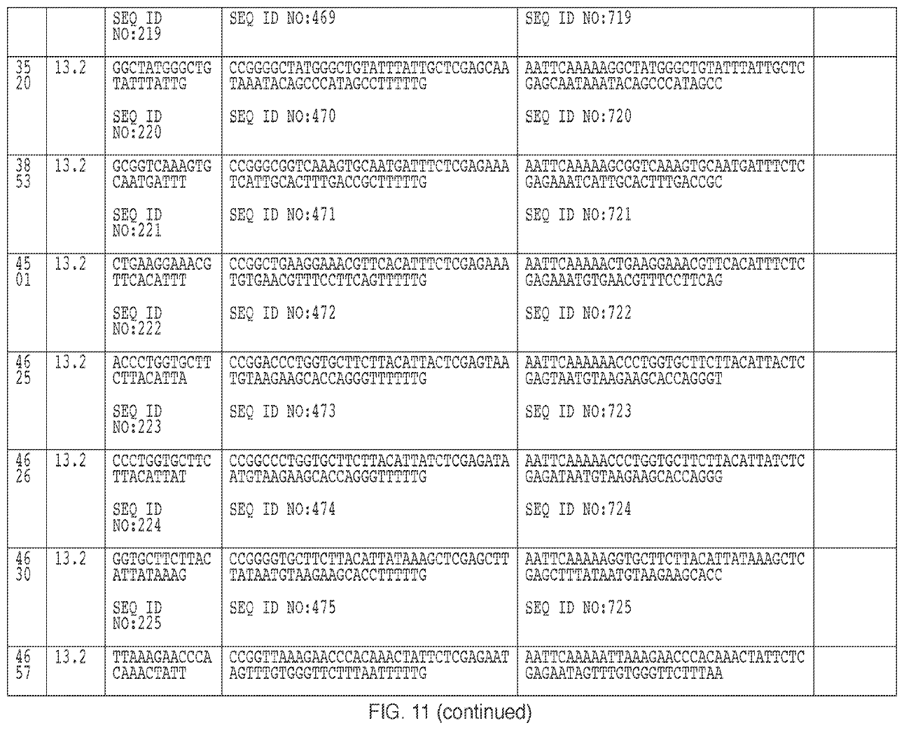

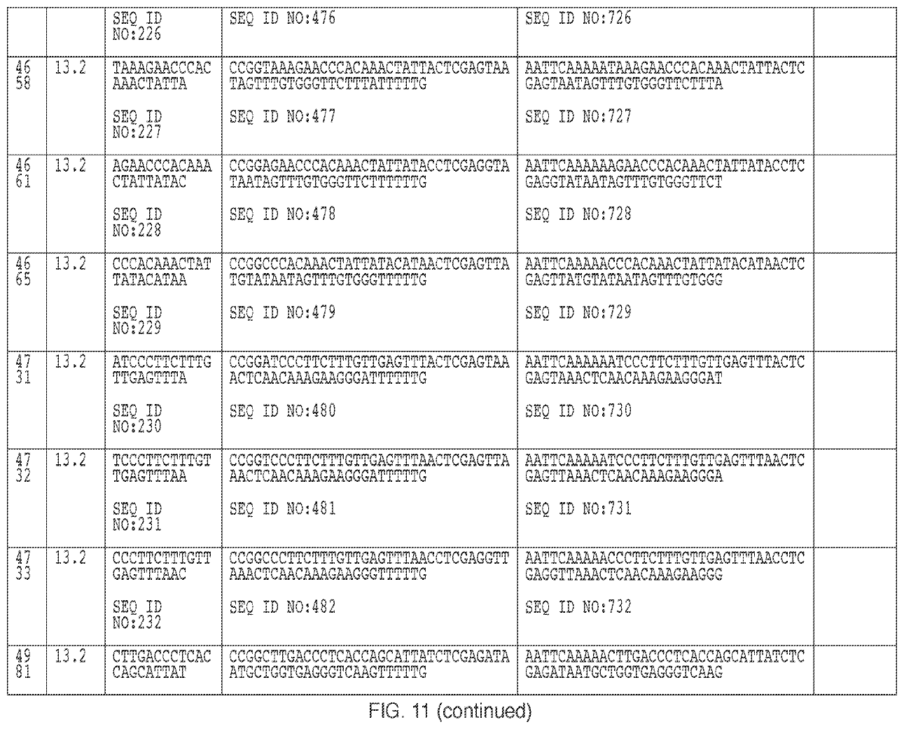

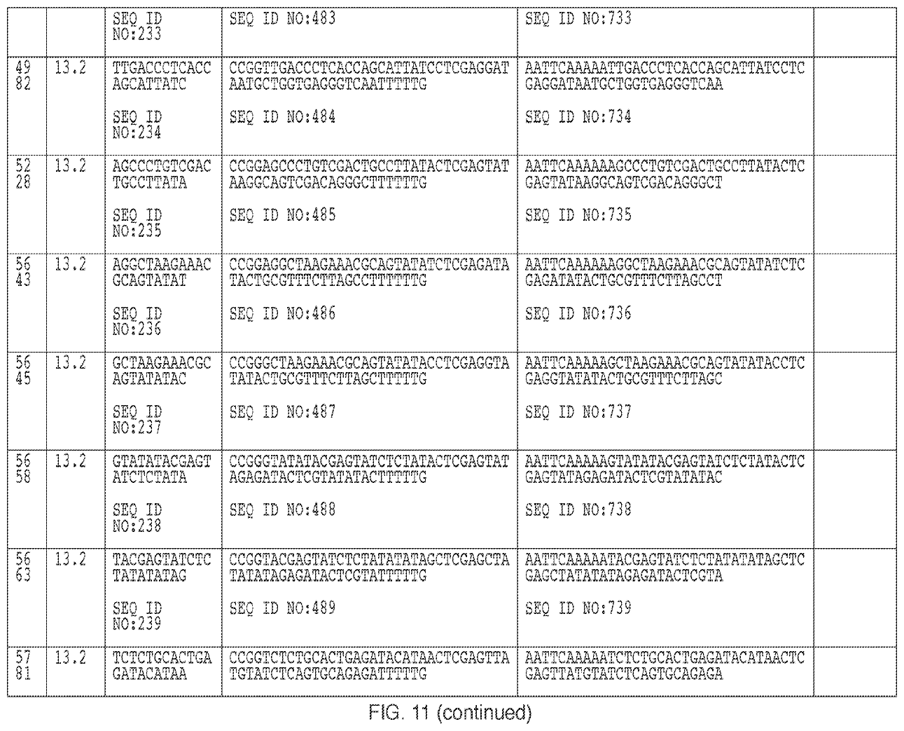

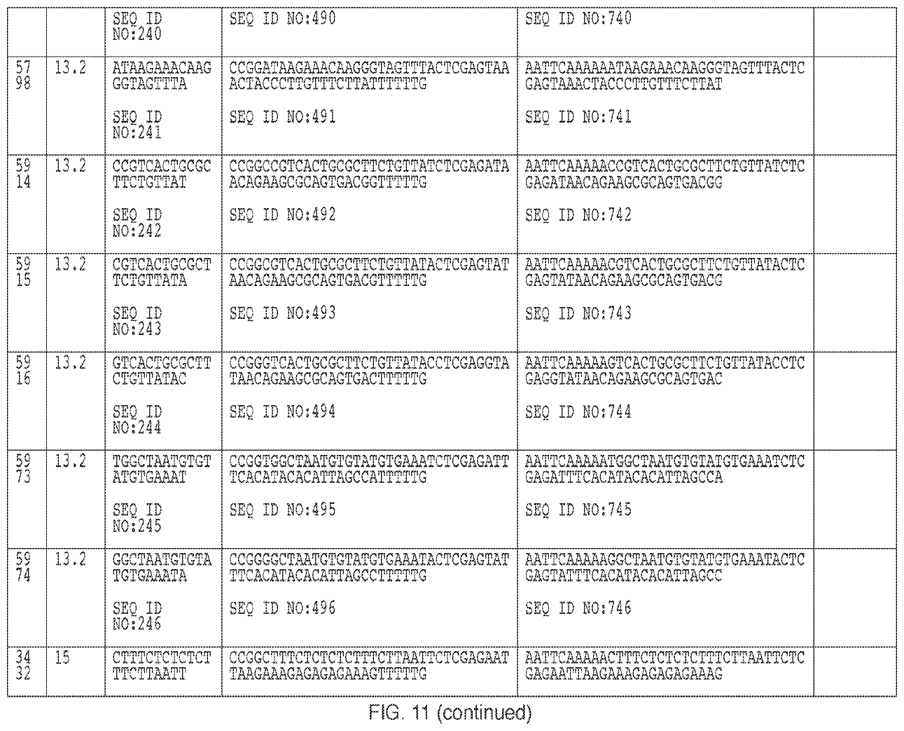

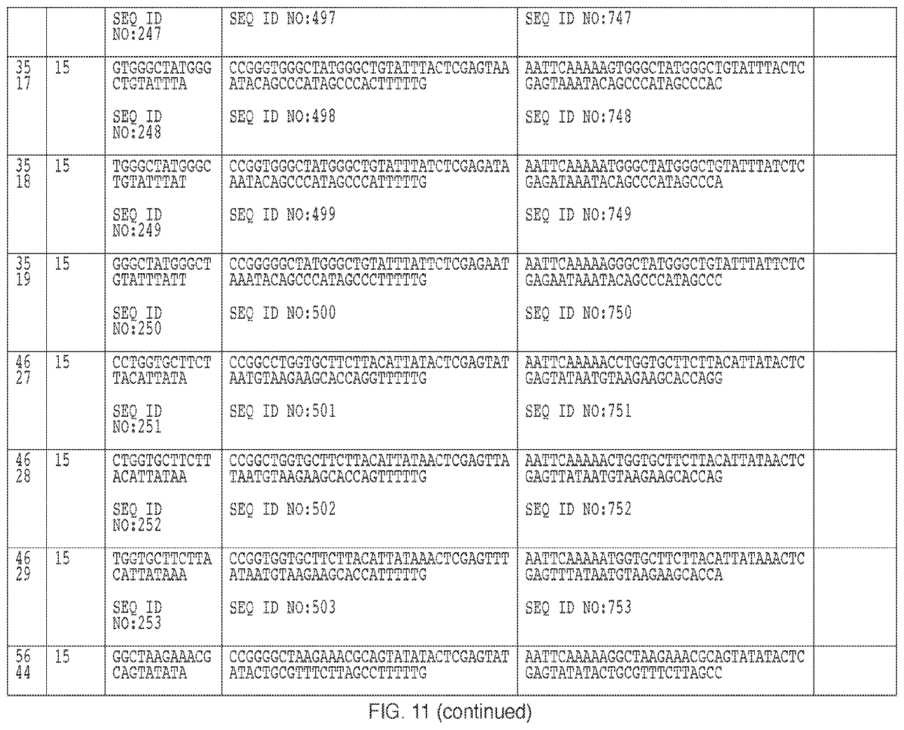

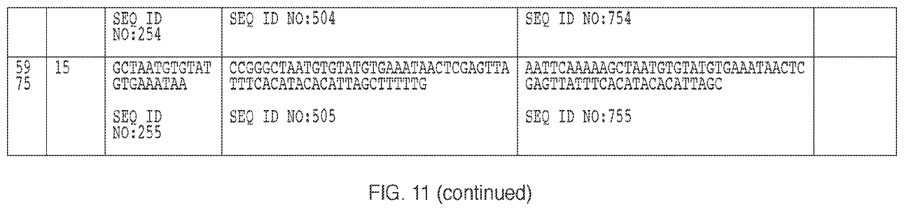

In some embodiments, an siRNA molecule targets the N-terminus of NFIX (e.g., within the sequence encoding the DNA-binding domain) or the promoter/regulatory region shown in FIG. 10. Example shRNA sequences targeting NFIX are found in Table 1:

| shRNA | Sequence |
|---|---|
| sh- | CCGGACATTGGAGTCACAATCAAAGCTCGAGCTTTGATTGTGACTCCAAT |
| NFIX | GTTTTTTG |
| #1 | (SEQ ID NO:_) |
| sh- | CCGGGGAATCCGGACAATCAGATAGCTCGAGCTATCTGATTGTCCGGATT |
| NFIX | CCTTTTTG |
| #2 | (SEQ ID NO:_) |
| | CCGGGCAGTCTCAGTCCTGGTTCCTCTCGAGAGGAACCAGGACTGAGACT |
| | GCTTTTT |
| | (SEQ ID NO:_) |
| | CCGGACTGGATCTTTATCTGGCTTACTCGAGTAAGCCAGATAAAGATCCA |
| | GTTTTTT |
| | (SEQ ID NO:_) |
| | CCGGCCGGCTTCTCTAAAGAAGTCACTCGAGTGACTTCTTTAGAGAAGCC |
| | GGTTTTT |
| | (SEQ ID NO:_) |
| | CCGGCACATCACATTGGAGTCACAACTCGAGTTGTGACTCCAATGTGATG |
| | TGTTTTT |
| | (SEQ ID NO:_) |
| | CCGGCCTGTTGATGACGTGTTCTATCTCGAGATAGAACACGTCATCAACA |
| | GGTTTTT |
| | (SEQ ID NO:_) |
| | CCGGATCTTTATCTGGCTTACTTTGCTCGAGCAAAGTAAGCCAGATAAAG |
| | ATTTTTTG |
| | (SEQ ID NO:_) |
| | CCGGTAGGCTAAGAAACGCAGTATACTCGAGTATACTGCGTTTCTTAGCC |
| | TATTTTTG (SEQ ID NO:_) |
| | CCGGCCCAACGGGCACTTAAGTTTCCTCGAGGAAACTTAAGTGCCCGTTG |
| | GGTTTTTG (SEQ ID NO:_) |

A viral vector based on any appropriate virus may be used to deliver a nucleic acid of the disclosure. In addition, hybrid viral systems may be of use. The choice of viral delivery system will depend on various parameters, such as the tissue targeted for delivery, transduction efficiency of the system, pathogenicity, immunological and toxicity concerns, and the like.

Commonly used classes of viral systems used in gene therapy can be categorized into two groups according to whether their genomes integrate into host cellular chromatin (retroviruses and lentiviruses) or persist in the cell nucleus predominantly as extrachromosomal episomes (adeno-associated virus, adenoviruses and herpesviruses). In one example, a viral vector integrates into a host cell's chromatin. In another example, a viral vector persists in a host cell's nucleus as an extrachomosomal episome.

In one example, a viral vector is from the Parvoviridae family. The Parvoviridae is a family of small single-stranded, non-enveloped DNA viruses with genomes approximately 5000 nucleotides long. Included among the family members is adeno-associated virus (AAV). In one example, a viral vector of the disclosure is an AAV. AAV is a dependent parvovirus that generally requires co-infection with another virus (typically an adenovirus or herpesvirus) to initiate and sustain a productive infectious cycle. In the absence of such a helper virus, AAV is still competent to infect or transduce a target cell by receptor-mediated binding and internalization, penetrating the nucleus in both non-dividing and dividing cells. Because progeny virus is not produced from AAV infection in the absence of helper virus, the extent of transduction is restricted only to the initial cells that are infected with the virus. Unlike retrovirus, adenovirus, and herpes simplex virus, AAV appears to lack human pathogenicity and toxicity (Kay et al., Nature 424:251, 2003). Since the genome normally encodes only two genes it is not surprising that, as a delivery vehicle, AAV is limited by a packaging capacity of 4.5 single stranded kilobases (kb). However, although this size restriction may limit the genes that can be delivered for replacement gene therapies, it does not adversely affect the packaging and expression of shorter sequences such as shRNA.

In one example, a viral vector is an adenoviral (AdV) vector. Adenoviruses are medium-sized double-stranded, non-enveloped DNA viruses with linear genomes that are between 26-48 kbp. Adenoviruses gain entry to a target cell by receptor-mediated binding and internalization, penetrating the nucleus in both non-dividing and dividing cells. Adenoviruses are heavily reliant on the host cell for survival and replication and are able to replicate in the nucleus of vertebrate cells using the host's replication machinery.

Another viral delivery system useful with the nucleic acid constructs of the disclosure is a system based on viruses from the family Retroviridae. Retroviruses comprise single-stranded RNA animal viruses that are characterized by two unique features. First, the genome of a retrovirus is diploid, consisting of two copies of the RNA. Second, the RNA is transcribed by the virion-associated enzyme reverse transcriptase into double-stranded DNA. This double-stranded DNA or provirus can then integrate into the host genome and be passed from parent cell to progeny cells as a stably-integrated component of the host genome.

In some examples, a viral vector is a lentivirus. Lentivirus vectors are often pseudotyped with vesicular steatites virus glycoprotein (VSV-G), and have been derived from the human immunodeficiency virus (HIV); visan-maedi, which causes encephalitis (visna) or pneumonia in sheep; equine infectious anemia virus (EIAV), which causes autoimmune hemolytic anemia and encephalopathy in horses; feline immunodeficiency virus (FIV), which causes immune deficiency in cats; bovine immunodeficiency virus (BIV) which causes lymphadenopathy and lymphocytosis in cattle; and simian immunodeficiency virus (SIV), which causes immune deficiency and encephalopathy in non-human primates. Vectors that are based on HIV generally retain <5% of the parental genome, and <25% of the genome is incorporated into packaging constructs, which minimizes the possibility of the generation of reverting replication-competent HIV. Biosafety has been further increased by the development of self-inactivating vectors that contain deletions of the regulatory elements in the downstream long-terminal-repeat sequence, eliminating transcription of the packaging signal that is required for vector mobilization. One of the main advantages to the use of lentiviral vectors is that gene transfer is persistent in most tissues or cell types, even following cell division of the transduced cell.

A lentiviral-based construct used to express a RNA of the disclosure comprises sequences from the 5′ and 3′ long terminal repeats (LTRs) of a lentivirus. In one example, the viral construct comprises an inactivated or self-inactivating 3′ LTR from a lentivirus. The 3′ LTR may be made self-inactivating by any method known in the art. For example, the U3 element of the 3′ LTR contains a deletion of its enhancer sequence, e.g., the TATA box, Sp1 and NF-kappa B sites. As a result of the self-inactivating 3′ LTR, the provirus that is integrated into the host genome will comprise an inactivated 5′ LTR. The LTR sequences may be LTR sequences from any lentivirus from any species. The lentiviral-based construct also may incorporate sequences for MMLV or MSCV, RSV or mammalian genes. In addition, the U3 sequence from the lentiviral 5′ LTR may be replaced with a promoter sequence in the viral construct. This may increase the titer of virus recovered from the packaging cell line. An enhancer sequence may also be included.

Other viral or non-viral systems known to those skilled in the art may be used to deliver the nucleic acid to cells of interest, including but not limited to gene-deleted adenovirus-transposon vectors (see Yant, et al., Nature Biotech. 20:999-1004, 2002); systems derived from Sindbis virus or Semliki forest virus (see Perri, et al., J. Virol. 74(20):9802-9807, 2002); systems derived from Newcastle disease virus or Sendai virus.

Antisense technology: Antisense nucleic acids are a class of nucleic acid-based compounds that can be used to inhibit an mRNA of NFIX comprising a cryptic exon. In case of doubt, the nucleic acid construct that inhibits the expression of NFIX and which can be incorporated into any of the compositions of the invention or used in the methods of treatment described herein can be, or can include, a nucleic acid sequence that is antisense to an NFIX sequence. Antisense nucleic acids may be single- or double-stranded deoxyribonucleic acid (DNA)-based, ribonucleic acid (RNA)-based, or DNA/RNA chemical analogue compounds. In general, antisense nucleic acids are designed to include a nucleotide sequence that is complementary or nearly complementary (e.g., at least 80% complimentary) to an mRNA (i.e., a mature, intronless mRNA) or pre-mRNA (i.e., a precursor mRNA containing introns) sequence transcribed from a given gene in order to promote binding between the antisense therapeutic and the pre-mRNA or mRNA. Without wishing to be bound by theory, it is believed that in most instances antisense therapeutics act by binding to an mRNA or pre-mRNA, thereby inhibiting protein translation, altering pre-mRNA splicing into mature mRNA, and/or causing destruction of mRNA. In most instances, the antisense therapeutic nucleotide sequence is complementary to a portion of a targeted gene's or mRNA's sense sequence. Nucleic acid constructs that inhibit the expression of NFIX include NFIX antisense oligonucleotides (ASOs) (e.g., single-stranded ASOs, NFIX shRNAs, NFIX siRNAs, and morpholino oligomers that impair NFIX mRNA translation into protein). The nucleic acid constructs useful within the compositions and methods described herein are not limited to those that inhibit NFIX by any particular mechanism of action. For example, a nucleic acid construct that inhibits the expression of NFIX may do so by binding directly to NFIX mRNA and obstructing protein translation, sterically blocking the ribosome complex, thereby inhibiting translation, activating RNase H to cleave and degrade NFIX mRNA in the ASO-NFIX mRNA complex, inhibiting 5′ cap formation, modulating RNA splicing, or blocking polyadenylation.

ASOs, useful in the compositions and methods described herein, comprise short oligonucleotide-based sequences that include an oligonucleotide sequence complementary to a target RNA sequence (here, in case of any doubt, NFIX). ASOs are typically between 8 to 50 nucleotides in length, for example, about 20 nucleotides in length (e.g. single-stranded oligonucleotides of 15-22 bp are typically efficiently taken up by cells), but may be as long as about 100 nucleotides (e.g., about 60 nucleotides). ASOs used in the present compositions and methods may include chemically modified nucleosides (for example, 2′-O-methylated nucleosides or 2′-O-(2-methoxyethyl) nucleosides) as well as modified internucleoside linkages (for example, phosphorothioate linkages).

Morpholino oligomers are oligonucleotide compounds that include DNA bases attached to a backbone of methylenemorpholine rings linked through phosphorodiamidate groups (see, e.g., PMID: 28252184 US991474582). Morpholino oligomers can be designed to bind to a specific NFIX sequence of interest, e.g., the N-terminus of NFIX.

Nuclease-editing systems: A nuclease genomic editing system can use a variety of nucleases to introduce a break or cut at a target genomic locus, including, without limitation, a Clustered Regularly Interspaced Short Palindromic Repeats (CRISPR) family nuclease or derivative thereof, a Transcription activator-like effector nuclease (TALEN) or derivative thereof, a zinc-finger nuclease (ZFN) or derivative thereof, and a homing endonuclease (HE) or derivative thereof. A derivative of any of the foregoing nucleases includes, for example, a mutant form, conjugated form, or otherwise modified form of the nuclease that retains sufficient functionality to inhibit NFIX to a therapeutically beneficial level.

In some embodiments, a CRISPR-mediated gene editing system can be used to engineer a host genome (i.e., a patient's genome) to encode an engineered nucleic acid, such as an engineered nucleic acid encoding one or more of the effector molecules described herein. CRISPR systems are described in more detail in Adli (Nature Communications, 9, Article number 1911, 2018). Generally, a CRISPR-mediated gene editing system comprises a CRISPR-associated (Cas) nuclease and an RNA that directs cleavage to a particular target sequence. An exemplary CRISPR-mediated gene editing system is the CRISPR/Cas9 system comprised of a Cas9 nuclease and an RNA comprising a CRISPR RNA (crRNA) domain and a trans-activating CRISPR (tracrRNA) domain. The crRNA typically has two RNA domains: a guide RNA sequence (gRNA) that directs specificity through base-pair hybridization to a target sequence (e.g., a genomic sequence) and an RNA domain that hybridizes to a tracrRNA. A tracrRNA can interact with and thereby promote recruitment of a nuclease (e.g., Cas9) to a genomic locus. The crRNA and tracrRNA polynucleotides can be separate polynucleotides. The crRNA and tracrRNA polynucleotides can be a single polynucleotide, also referred to as a single guide RNA (sgRNA). If necessary or desired, one of ordinary skill in the art can utilize publicly available services and websites to aid in the design of sgRNAs useful in inhibiting the expression of NFIX. For example, one can utilize the GPP sgRNA Designer available at The Broad Institute (www.portals.broadinstitute.org/gpp/public/analysis-tools/sgrna-design; see also, Doench et al., Nature Biotechnol. 34(2):184-191, 2016), CHOPCHOP (www.chopchop.cbu.uib.no/; see also Montague et al., Nucleic Acids Res. 42:W401-407, 2014), CRISPR RGEN Cas-designer (www.rgenome.net/cas-designer/; see also Park et al., Bioinformatics 31:4014-4016, 2015), E-CRISP (www.ecrisp.org/E-CRISP/designcrispr.html; see also Heigwer et al., Nature Methods 11(2):122-123, 2014), CRISPR MultiTargeter (www.multicrispr.net/; developed by Sergey Prykhozhij at the IWK Health Centre and Dalhousie University), Synthego CRISPR Design Tool (www.design.synthego.com/#/), and Benchling CRISPR Guide RNA design (www.benchling.com/crispr). Other CRISPR systems can be used, such as the Cpf1 system. Nucleases can include derivatives thereof, such as Cas9 functional mutants, e.g., a Cas9 “nickase” mutant that in general mediates cleavage of only a single strand of a defined nucleotide sequence as opposed to a complete double-stranded break typically produced by Cas9 enzymes.

In some embodiments, the components of a CRISPR system interact with each other to form a Ribonucleoprotein (RNP) complex to mediate sequence specific cleavage. In some CRISPR systems, each component can be separately produced and used to form the RNP complex. In some CRISPR systems, each component can be separately produced in vitro and contacted (i.e., “complexed”) with each other in vitro to form the RNP complex. The in vitro produced RNP can then be introduced into a cell by a variety of means including, without limitation, electroporation, lipid-mediated transfection, cell membrane deformation by physical means, lipid nanoparticles (LNP), virus like particles (VLP), and sonication. In a particular example, in vitro produced RNP complexes can be delivered to a cell using a Nucleofactor/Nucleofection® electroporation-based delivery system (Lonza®). Other electroporation systems include, without limitation, MaxCyte® electroporation systems, Miltenyi CliniMACS® electroporation systems, Neon electroporation systems, and BTX® electroporation systems. CRISPR nucleases, e.g., Cas9, can be produced in vitro (i.e., synthesized and purified) using a variety of protein production techniques known to those skilled in the art. CRISPR system RNAs, e.g., an sgRNA, can be produced in vitro (i.e., synthesized and purified) using a variety of RNA production techniques known to those skilled in the art, such as in vitro transcription or chemical synthesis.

In some CRISPR systems, each component (e.g., Cas9 and an sgRNA) can be separately encoded by a polynucleotide with each polynucleotide introduced into a cell together or separately. In some CRISPR systems, each component can be encoded by a single polynucleotide (e.g., a multi-promoter or multicistronic vector) and introduced into a cell. Following expression of each polynucleotide encoded CRISPR component within a cell (e.g., translation of a nuclease and transcription of CRISPR RNAs), an RNP complex can form within the cell and proceed to direct site-specific cleavage.

Some RNPs can be engineered to have moieties that promote delivery of the RNP into the nucleus. For example, a Cas9 nuclease can have a nuclear localization signal (NLS) domain such that if a Cas9 RNP complex is delivered into a cell's cytosol or following translation of Cas9 and subsequent RNP formation, the NLS can promote further trafficking of a Cas9 RNP into the nucleus.

The engineered cells described herein can be engineered using non-viral methods, e.g., the nuclease and/or CRISPR mediated gene editing systems described herein can be delivered to a cell using non-viral methods. The engineered cells described herein can be engineered using viral methods, e.g., the nuclease and/or CRISPR mediated gene editing systems described herein can be delivered to a cell using viral methods such as adenoviral, retroviral, lentiviral, or any of the other viral-based delivery methods described herein. In some embodiments, CRISPR target the NFIX gene locus on 19p13.13 (RefSeq DNA Sequence ID NC_000019.10).

In some CRISPR systems, more than one CRISPR composition can be provided such that each composition separately targets the same gene or general genomic locus at more than one target nucleotide sequence. For example, two separate CRISPR compositions can be provided to direct cleavage at two different target nucleotide sequences within a certain distance of each other. In some CRISPR systems, more than one CRISPR composition can be provided such that each separately target opposite strands of the same gene or general genomic locus. For example, two separate CRISPR “nickase” compositions can be provided to direct cleavage at the same gene or general genomic locus at opposite strands.

In general, the features of a CRISPR-mediated editing system described herein can apply to other nuclease-based genomic editing systems. TALEN is an engineered site-specific nuclease, which is composed of the DNA-binding domain of TALE (transcription activator-like effectors) and the catalytic domain of restriction endonuclease Fokl. By changing the amino acids present in the highly variable residue region of the monomers of the DNA binding domain, different artificial TALENs can be created to target various nucleotides sequences. The DNA binding domain subsequently directs the nuclease to the target sequences and creates a double-stranded break. TALEN-based systems are described in more detail in U.S. Ser. No. 12/965,590; U.S. Pat. Nos. 8,450,471; 8,440,431; 8,440,432; 10,172,880; and U.S. Ser. No. 13/738,381, all of which are incorporated by reference herein in their entirety. ZFN-based editing systems are described in more detail in U.S. Pat. Nos. 6,453,242; 6,534,261; 6,599,692; 6,503,717; 6,689,558; 7,030,215; 6,794,136; 7,067,317; 7,262,054; 7,070,934; 7,361,635; 7,253,273; and U.S. Patent Publication Nos. 2005/0064474; 2007/0218528; 2005/0267061, all incorporated herein by reference in their entireties.

Inducible Systems: A promoter may be one naturally associated with a gene or sequence, as may be obtained by isolating the 5′ non-coding sequences located upstream of the coding segment of a given gene or sequence. Such a promoter can be referred to as “endogenous.” In some embodiments, a coding nucleic acid sequence may be positioned under the control of a recombinant or heterologous promoter, which refers to a promoter that is not normally associated with the encoded sequence in its natural environment. Such promoters may include promoters of other genes, promoters isolated from another cell, and synthetic promoters or enhancers that are not “naturally occurring” such as, for example, those that contain different elements of different transcriptional regulatory regions and/or mutations that alter expression through methods of genetic engineering that are known in the art. In addition to producing nucleic acid sequences of promoters and enhancers synthetically, sequences may be produced using recombinant cloning and/or nucleic acid amplification technology, including polymerase chain reaction (PCR) (see, e.g., U.S. Pat. Nos. 4,683,202 and 5,928,906).

Promoters of an engineered nucleic acid may be “inducible promoters,” which refer to promoters that are characterized by regulating (e.g., activating) transcriptional activity when in the presence of, influenced by, or contacted with a signal. The signal may be endogenous or a normally exogenous condition (e.g., light), a compound (e.g., chemical or non-chemical compound), or a protein (e.g., cytokine or hormone) that contacts an inducible promoter in such a way as activate transcriptional activity from the inducible promoter. Activation of transcription may involve directly acting on a promoter to drive transcription or indirectly acting on a promoter by inactivation a repressor that is preventing the promoter from driving transcription. Conversely, deactivation of transcription may involve directly acting on a promoter to prevent transcription or indirectly acting on a promoter by activating a repressor that then acts on the promoter.

The inducible promoter used in the present methods of inhibiting NFIX can be one commonly used in the art, including any one or more of those highlighted in Table 2 below.

TABLE 2
Exemplary inducible promoter systems

| | Promoter and | Transcription | |
|---|---|---|---|
| System | operator | factor (TF) | Inducer molecule |
| AIR | PAIR (OalcA- PhCMVmin) | AlcR | Acetaldehyde |
| ART | PART (OARG- PhCMVmin) | ArgR-VP16 | 1-Arginine |
| BIT | PBIT3 (OBirA3- | BIT (BirA- | Biotin |
| | PhCMVmin) | VP16) | |
| Cumate- | PCR5 (OCu06- | cTA (CymR- | Cumate |
| activator | PhCMVmin) | VP16) | |
| Cumate- | PCR5 (OCuO6- | rcTA (rCymR- | Cumate |
| reverse activator | PhCMVmin) | VP16) | |
| E-OFF | PETR (OETR- PhCMVmin) | ET (E-VP16) | Erythromycin |
| NICE-OFF | PNIC (ONIC- | NT (HdnoR- | 6-Hydroxy-nicotine |
| | PhCMVmin) | VP16) | |
| PEACE | PTtgR1 (OTtgR- | TtgA1 (TtgR- | Phioretin |
| | PhCMVmin) | VP16) | |
| PIP-OFF | PPIR (OPIR- | PIT (PIP- | Pristinamycin I |
| | Phsp70min) | VP 16) | |
| QuoRex | PSCA (OscbR- | SCA (ScbR- | SCB1 |
| | PhCMVmin)PSPA | VP16) | |
| | (OpapRI- | | |
| | PhCMVmin) | | |
| Redox | PROP (OROP- | REDOX (REX- | NADH |
| | PhCMVmin) | VP 16) | |
| TET-OFF | PhCMV*-1 (OtetO7- | tTA (TetR- | Tetracycline |
| | PhCMVmin) | VP16) | |
| TET-ON | PhCMV*-1 (OtetO7- | rtTA (rTetR- | Doxycycline |
| | PhCMVmin) | VP16) | |
| TIGR | PCTA (OrheO- | CTA (RheA- | Heat |
| | PhCMVmin) | VP16) | |
| TraR | O7x(tra box)- PhCMVmin | p65-TraR | 3-Oxo-C8-HSL |
| VAC-OFF | P1VanO2 (OVanO2- | VanA1 (VanR- | Vanillic acid |
| | PhCMVmin) | VP16) | |
| Cumate- | PCuO | CymR | Cumate |
| repressor | (PCMV5-OCuO) | | |
| E-ON | PETRON8 | E-KRAB | Erythromycin |
| | (PSV40-OETR8) | | |
| NICE-ON | PNIC | NS (HdnoR- | 6-Hydroxy-nicotine |
| | (PSV40-ONIC8) | KRAB) | |
| PIP-ON | PPIRON | PIT3 (PIP- | Pristinamycin I |
| | (PSV40-OPIR3) | KRAB) | |
| Q-ON | PSCAON8 | SCS (ScbR- | SCB1 |
| | (PSV40-OscbR8) | KRAB) | |
| TET-ON, | OtetO-PHPRT | tTS-H4 (TetR- | Doxycycline |
| repressor- based | | HDAC4) | |
| T-REX | PTetO ( | TetR | Tetracycline |
| | PhCMV-OtetO2) | | |
| UREX | PUREX8 | mUTS (KRAB- | Uric acid |
| | (PSV40-OhucO8) | HucR) | |
| VAC-ON | PVanON8 (PhCMV- | VanA4 (VanR- | Vanillic acid |
| | OVanO8) | KRAB) | |
| QuoRexPIP- | OscbR8-OPIR3- | SCAPIT3 | SCB1Pristinamycin |
| ON(NOT IF | PhCMVmin | | I |
| gate) | | | |
| QuoRexE- | OscbR-OETR8- | SCAE-KRAB | SCB1Erythromycin |
| ON(NOT IF | PhCMVmin | | |
| gate) | | | |
| TET-OFFE- | OtetO7-OETR8- | tTAE-KRAB | Tetracycline- |
| ON(NOT IF | PhCMVmin | | Erythromycin |
| gate) | | | |
| TET- | OtetO7- | tTAPIT3E- | Tetracycline- |
| OFFPIP- | OPIR3-OETR8- | KRAB | Pristinamycin |
| ONE-ON | PhCMVmin | | IErythromycin |

Alternatively, a promoter may be specific to the tissue of interest, e.g., the erythrocyte lineage. In one embodiment, erythrocyte-specific expression is achieved by using the human β-globin promoter region and locus control region (LCR). In one embodiment, the human anykyrin-1 promoter region and locus control region (LCR) is used. In one embodiment, the human spectrin promoter region and locus control region (LCR) is used. It is further contemplated that a novel tissue-specific promoter for the erythrocyte lineage could be engineered using a unique marker of the cell type. Additional erythrocyte-specific promoters are presented in Moreau-Gaudry et al. (Blood 98(9):2664-2672, 2001).

Other gene-editing systems: Gene editing can be used to engineer a host genome to encode a nucleic acid, such as an engineered nucleic acid encoding one or more of the effector molecules described herein. In general, a “genomic editing system” refers to any system for integrating an exogenous gene into a host cell's genome. Genomic editing systems include, without limitation, a transposon system, a nuclease gene editing system, base-editing systems, prime-editing systems, and viral vector-based delivery platform.

A transposon system can be used to integrate an engineered nucleic acid, such as an engineered nucleic acid encoding one or more of the effector molecules described herein, into a host genome. Transposons generally comprise terminal inverted repeats (TIR) that flank a cargo/payload nucleic acid and a transposase. The transposon system can provide the transposon in cis or in trans with the TIR-flanked cargo. A transposon system can be a retrotransposon system or a DNA transposon system. In general, transposon systems integrate a cargo/payload (e.g., an engineered nucleic acid) randomly into a host genome. Examples of transposon systems include systems using a transposon of the Tc1/mariner transposon superfamily, such as a Sleeping Beauty transposon system, described in more detail in Hudecek et al. (Crit. Rev. Biochem. Mol. Biol. 52(4):355-380, 2017) and U.S. Pat. Nos. 6,489,458, 6,613,752 and 7,985,739, each of which is herein incorporated by reference herein. Another example of a transposon system includes a PiggyBac transposon system, described in more detail in U.S. Pat. Nos. 6,218,185 and 6,962,810, each of which is herein incorporated by reference herein.

A nuclease genomic editing system can be used to engineer a host genome to encode an engineered nucleic acid, such as an engineered nucleic acid encoding one or more of the effector molecules described herein. Without wishing to be bound by theory, in general, the nuclease-mediated gene editing systems used to introduce an exogenous gene take advantage of a cell's natural DNA repair mechanisms, particularly homologous recombination (HR) repair pathways. Briefly, following damage to genomic DNA (e.g., a double-stranded break), a cell can repair by using another DNA source with identical, or substantially identical, sequences at both its 5′ and 3′ ends as a template during DNA synthesis to repair the lesion. In a natural context, HR can use the other chromosome present in a cell as a template. In gene editing systems, exogenous polynucleotides are introduced into the cell to be used as a homologous recombination template (HRT or HR template). In general, any additional exogenous sequence not originally found in the chromosome with the lesion that is included between the 5′ and 3′ complimentary ends within the HRT (e.g., a gene or a portion of a gene) can be incorporated (i.e., “integrated”) into the given genomic locus during templated HR. Thus, a typical HR template for a given genomic locus has a nucleotide sequence identical to a first region of an endogenous genomic target locus, a nucleotide sequence identical to a second region of the endogenous genomic target locus, and a nucleotide sequence encoding a cargo/payload nucleic acid (e.g., any of the engineered nucleic acids described herein, such as any of the engineered nucleic acids encoding one or more effector molecules).

In some embodiments, a HR template can be linear. Examples of linear HR templates include, without limitation, a linearized plasmid vector, a ssDNA, a synthesized DNA, and a PCR amplified DNA. In some embodiments, a HR template can be circular, such as a plasmid. A circular template can include a supercoiled template.

The identical, or substantially identical, sequences found at the 5′ and 3′ ends of the HR template, with respect to the exogenous sequence to be introduced, are generally referred to as arms (HR arms). HR arms can be identical to regions of the endogenous genomic target locus (i.e., 100% identical). HR arms in some examples can be substantially identical to regions of the endogenous genomic target locus. While substantially identical HR arms can be used, it can be advantageous for HR arms to be identical as the efficiency of the HDR pathway may be impacted by HR arms having less than 100% identity.

Lipid-based delivery: Nucleic acids (e.g., any of the engineered nucleic acid constructs described herein) can be introduced into a cell using a lipid-mediated delivery system, which generally uses a structure having an outer lipid membrane enveloping an internal compartment. Lipid-based structures useful in the methods of the invention include, without limitation, lipid-based nanoparticles, liposomes, micelles, exosomes, vesicles, or extracellular vesicles. Lipid-mediated delivery systems can deliver a cargo (e.g., any of the engineered nucleic acids described herein) in vitro, in vivo, or ex vivo.

A liposome used according to the present embodiments can be made by different methods, as would be known to one of ordinary skill in the art. Preparations of liposomes are described in further detail in WO 2016/201323, International Applications PCT/US85/01161 and PCT/US89/05040, and U.S. Pat. Nos. 4,728,578, 4,728,575, 4,737,323, 4,533,254, 4,162,282, 4,310,505, and 4,921,706; each herein incorporated by reference for all purposes.

Liposomes can be cationic liposomes. Examples of useful cationic liposomes are described in more detail in U.S. Pat. Nos. 5,962,016; 5,030,453; 6,680,068, U.S. Application 2004/0208921, and International Patent Applications WO03/015757A1, WO04029213A2, and WO02/100435A1, each hereby incorporated by reference in their entirety.

Lipid-mediated gene delivery methods are described, for instance, in WO 96/18372; WO 93/24640; Mannino & Gould-Fogerite, BioTechniques 6(7):682-691, 1988; U.S. Pat. Nos. 5,279,833; 5,279,833; WO 91/06309; and Felgner et al., Proc. Natl. Acad. Sci. USA 84:7413-7414, 1987, each US Patent being incorporated by reference herein.

As used herein, the term “extracellular vesicle” or “EV” refers to a cell-derived vesicle comprising a membrane that encloses an internal space. In general, extracellular vesicles comprise all membrane-bound vesicles with smaller diameter than the cell from which they are derived. Generally extracellular vesicles range in diameter from 20 nm to 1000 nm and can comprise various macromolecular cargo either within the internal space, displayed on the external surface of the extracellular vesicle, and/or spanning the membrane. The cargo can comprise nucleic acids (e.g., any of the engineered nucleic acids described herein), proteins, carbohydrates, lipids, small molecules, and/or combinations thereof. By way of example and without limitation, extracellular vesicles include apoptotic bodies, fragments of cells, vesicles derived from cells by direct or indirect manipulation (e.g., by serial extrusion or treatment with alkaline solutions), vesiculated organelles, and vesicles produced by living cells (e.g., by direct plasma membrane budding or fusion of the late endosome with the plasma membrane). Extracellular vesicles can be derived from a living or dead organism, explanted tissues or organs, and/or cultured cells.

Lipid nanoparticles (LNPs), in general, are synthetic lipid structures that rely on the amphiphilic nature of lipids to form membranes and vesicle like structures (Riley and Vermerris, Nanomaterials, 7:94, 2017). In general, these vesicles deliver cargo/payloads, such as any of the engineered nucleic acids or viral systems described herein (i.e., the nucleic acid constructs that inhibit the expression of NFIX), by absorbing into the membrane of target cells and releasing the cargo into the cytosol. Lipids used in LNP formation can be cationic, anionic, or neutral. The lipids can be synthetic or naturally derived, and in some instances biodegradable. Lipids can include fats, cholesterol, phospholipids, lipid conjugates including, but not limited to, polyethyleneglycol (PEG) conjugates (PEGylated lipids), waxes, oils, glycerides, and fat-soluble vitamins. Lipid compositions generally include defined mixtures of materials, such as the cationic, neutral, anionic, and amphipathic lipids. In some instances, specific lipids are included to prevent LNP aggregation, prevent lipid oxidation, or provide functional chemical groups that facilitate attachment of additional moieties. Lipid composition can influence overall LNP size and stability. In an example, the lipid composition comprises dilinoleylmethyl-4-dimethylaminobutyrate (MC3) or MC3-like molecules. MC3 and MC3-like lipid compositions can be formulated to include one or more other lipids, such as a PEG or PEG-conjugated lipid, a sterol, or neutral lipids. In addition, LNPs can be further engineered or functionalized to facilitate targeting of specific cell types. Another consideration in LNP design is the balance between targeting efficiency and cytotoxicity.

Micelles, in general, are spherical synthetic lipid structures that are formed using single-chain lipids, where the single-chain lipid's hydrophilic head forms an outer layer or membrane and the single-chain lipid's hydrophobic tails form the micelle center. Micelles typically refer to lipid structures only containing a lipid mono-layer. Micelles are described in more detail in Quader et al. (Mol Ther. 25(7):1501-1513, 2017), which can provide guidance as needed, together with additional information known in the art, where a micelle is used in the compositions and methods described herein.

Nanomaterials: Nanomaterials can be used to deliver engineered nucleic acids (e.g., any of the engineered nucleic acids described herein). Nanomaterial vehicles can be made of non-immunogenic materials and generally avoid eliciting immunity to the delivery vector itself. These materials can include, but are not limited to, lipids, inorganic nanomaterials, and other polymeric materials. Nanomaterial particles are described in more detail in Riley et al. (Nanomaterials 7(5):94, 2017).

Compositions and methods for delivering mRNAs in vivo, such as naked plasmids or mRNA, are described in detail in Kowalski et al. (Mol Ther. 27(4):710-728, 2019) and Kaczmarek et al. (Genome Med. 9:60, 2017).

Electroporation: In some embodiments, a nucleic acid described herein is introduced into a cell or other target recipient ex vivo. Electroporation can be used to deliver polynucleotides to recipients. Briefly, electroporation is a method of internalizing a cargo into a target cell or other entity through application of an electrical field to transiently permeabilize the outer membrane or shell of the target cell or entity. In the example of cells, it is believed that a fraction of the cells remain viable. Cells and other target entities can be electroporated in vitro, in vivo, or ex vivo. A variety of commercial devices and protocols can be used for electroporation, such as, without limitation, Neon® Transfection System, MaxCyte® Flow Electroporation™, Lonza® Nucleofector™ systems, and Bio-Rad® electroporation systems.

Additional methods for introducing engineered nucleic acids (e.g., any of the engineered nucleic acids described herein) into a cell or other target entity include, without limitation, sonication, gene gun, hydrodynamic injection, and cell membrane deformation by physical means such as compression.

Targeting of cellular recipients: In some embodiments, cell populations for transduction and administration of the nucleic acids described herein can be targeted in vivo or ex vivo. Cells comprising a nucleic acid construct described herein can be administered through a parenteral route to the subject in need thereof and may include a pharmaceutically acceptable carrier (e.g., a medium compatible with cellular viability). In some embodiments, cells comprising the nucleic acid construct may be injected directly into a blood vessel, such as vein, artery, venule or arteriole, via, e.g., hydrodynamic injection or catheterization. Administration may be by a single injection or by two or more injections.

In some embodiments, ex vivo targeting of cells comprises expansion of the donor cells, transduction of the cells with a nucleic acid described herein, expansion of the transduced donor cells, and administration of the transduced donor cells to the subject in need thereof. In some embodiments, the ex vivo targeting further comprises a selection for cells transduced with the nucleic acid. Selection techniques are known in the art and include, without limitation, antibiotic selection and fluorescent protein expression.

Cell populations: Cells that can be genetically modified as described herein include, without limitation, stem cells (e.g., hematopoietic stem cells (HSCs)), progenitor cells (e.g., hematopoietic (e.g., myeloid or erythroid) progenitor cells (HPCs or EPCs)), erythroblasts (EBs) at various stages of development prior to enucleation, and combinations thereof (e.g., a population of cells may include cells at any one or more of these stages of differentiation). The cells to be treated (e.g., transduced) can be obtained from any tissue in which they normally reside (e.g., bone marrow, umbilical cord, placenta, mesenchyme, or blood (e.g., peripheral blood)), and the methods of treating a patient as described herein can include a step of obtaining any one or more of the cells described herein from a source (e.g., isolating HSCs, HPCs, or EBs from bone marrow, umbilical cord blood, or a blood vessel). As noted, the invention also encompasses non-naturally occurring, genetically modified cells in which the level of NFIX expression is decreased relative to that of a comparable, non-genetically modified cell of the same type, and that cell type may be any described here. Such cells or populations of cells may also be isolated cells or isolated populations of cells. In the methods of treatment, the cells administered to a patient having a hemoglobinopathy can be autologous (“self”) or non-autologous (“non-self” e.g., allogeneic, syngeneic or xenogeneic).

HSCs derived from the bone marrow (also known as bone marrow-derived stem cells, BMDSCs), give rise to committed HPCs that can generate the entire repertoire of mature blood cells over the lifetime of an organism. As noted above, cells within the scope of the present invention and useful in the methods described herein may vary in their state of differentiation, and one of ordinary skill in the art will recognize the transition of one cell type to another. For example, erythropoiesis is known as the process of differentiation by which a multipotent stem cell (e.g., a multipotent HPC) transitions to a terminally differentiated cell (e.g., an erythrocyte). Stages of differentiation in erythropoiesis include: multipotential hematopoietic stem cell, common myeloid progenitor, proerythroblast (pronormoblast), basophilic erythroblast, polychromatic erythroblast, and orthochromatic erythroblast (normoblast). Cell stage can be determined using techniques known in the art to identify the presence or absence of specific markers, e.g., flow cytometry, and any one or more of the cell types just referenced may be selected for genetic modification and/or administration to a patient, as described herein. If desired, one can characterize a cell (e.g., a hematopoietic progenitor cell (e.g., of the erythroid lineage) as having at least one of the following cell surface markers, which are characteristic of hematopoietic progenitor cells: CD34+, CD36, CD59+, CD71, CD133, Terl19, Thy1/CD90+, CD38^{lo/-}, and C-kit/CD117+. These markers can be assessed using techniques known in the art, e.g., flow cytometry.

Hemoglobinopathies: Patients treated according to the methods described herein can be suffering from a hemoglobinopathy or be suspected of having or at risk of developing a hemoglobinopathy. As noted, the hemoglobinopathy can be sickle cell disease (also known as sickle cell anemia (e.g., HbSC, HbSD, HbSE, or HbSO)) or β-thalassemia (also known as Cooley's anemia or Mediterranean anemia). In some instances, the thalassemia can be δβ-thalassemia, in which both the delta and beta globin genes are affected. If desired, hemoglobinopathies can be further characterized based on their severity. For example, a patient diagnosed with β-thalassemia may have thalassemia major or thalassemia intermedia. One of ordinary skill in the art is able to diagnose and characterize hemoglobinopathies.

Kits: In some embodiments, provided herein are kits. The kits may include a pharmaceutical composition as described herein, in suitable packaging, and written material that can include instructions for use (e.g., in treating a patient having a hemaglobinopathy), discussion of clinical studies, listing of side effects, and the like. Such kits may also include information, such as scientific literature references, package insert materials, clinical trial results, and/or summaries of these and the like, which indicate or establish the activities and/or advantages of the pharmaceutical composition, and/or which describe dosing, administration, side effects, drug interactions, or other information useful to the health care provider. Such information may be based on the results of various studies, for example, studies using experimental animals involving in vivo models and studies based on human clinical trials.

A genetically modified cell that is incorporated within a composition of the invention or used according to a method described herein (e.g., a hematopoietic stem cell, a hematopoietic progenitor cell, or an erythroblast) will have a decreased level of NFIX expression relative to that of a comparable, non-genetically modified cell of the same type. In some embodiments, the kit comprises the genetically modified cell in a pharmaceutically acceptable composition. Kits described herein contain instructions for use. Suitable packaging and additional articles for use (e.g., a measuring cup or other vessel for handling or transferring liquid preparations, foil wrapping to minimize exposure to air, and the like) are known in the art and may be included in the kit. In other embodiments, kits may further comprise devices that are used to administer the active agents. Examples of such devices include, but are not limited to, syringes, drip bags, patches, and inhalers. Kits described herein may be provided, marketed and/or promoted to health providers, including physicians, nurses, pharmacists, formulary officials, and the like. Kits can also, in some embodiments, be marketed directly to the consumer.

Kits may further comprise pharmaceutically acceptable vehicles that may be used to administer one or more active agents (i.e., a nucleic acid construct that inhibits the expression of NFIX). For example, if an active agent is provided in a solid form (e.g., frozen or desiccated) that must be reconstituted for parenteral administration, the kit can comprise a sealed container of a suitable vehicle in which the active agent may be dissolved or otherwise treated to form a composition (e.g., a particulate-free sterile solution) that is suitable for parenteral administration. Examples of pharmaceutically acceptable vehicles include, but are not limited to: Water for Injection USP, aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; non-aqueous vehicles such as, but not limited to, corn oil, cottonseed oil, peanut oil, sesame oil, ethyl oleate, isopropyl myristate, and benzyl benzoate; and cell or tissue culture media.

The present disclosure further encompasses anhydrous pharmaceutical compositions and dosage forms comprising an active ingredient, since water can facilitate the degradation of some compounds. For example, water may be added (e.g., about 5%) in the pharmaceutical arts as a means of simulating long-term storage in order to determine characteristics such as shelf-life or the stability of formulations over time. Anhydrous pharmaceutical compositions and dosage forms may be prepared using anhydrous or low moisture containing ingredients and low moisture or low humidity conditions. For example, pharmaceutical compositions and dosage forms which contain lactose may be made anhydrous if substantial contact with moisture and/or humidity during manufacturing, packaging, and/or storage is expected. An anhydrous pharmaceutical composition may be prepared and stored such that its anhydrous nature is maintained. Accordingly, anhydrous pharmaceutical compositions may be packaged using materials known to prevent exposure to water such that they may be included in suitable formulary kits. Examples of suitable packaging include, but are not limited to, hermetically sealed foils, plastic or the like, unit dose containers, blister packs, and strip packs.

### EXAMPLES

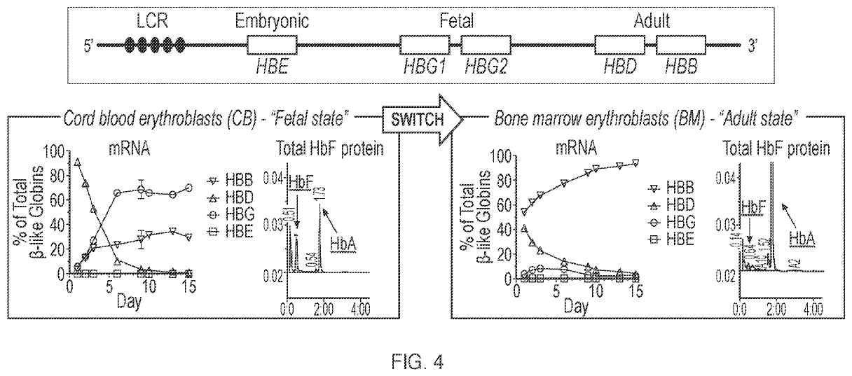

Fetal and adult state erythroblasts were used to identify a new HbF repressor: Red blood cells carry oxygen using hemoglobin, which is encoded by genes that are developmentally regulated during the embryonic, fetal, and adult stages of life. In human cord blood-derived erythroblasts (CB) and in neonates, gamma globin (HBG) gene expression is predominant, which corresponds to higher fetal hemoglobin or HbF, as shown in FIG. 4. A few months after birth, fetal-to-adult hemoglobin switching occurs; the gamma globin genes are silenced and expression of the adult form gene, HBB, is predominant. This fetal-to-adult hemoglobin switching, leading to the silencing of fetal hemoglobin, is of interest because induction of fetal hemoglobin can ameliorate the symptoms associated with hemoglobinopathies such as SCD and β-thalassemias. To identify novel lineage- and stage-specific repressors of fetal hemoglobin RNA-seq and ATAC-seq (assay for transposase-accessible chromatin) were performed on fetal and adult state CB and erythroblasts derived from adult bone marrow (BM), respectively.

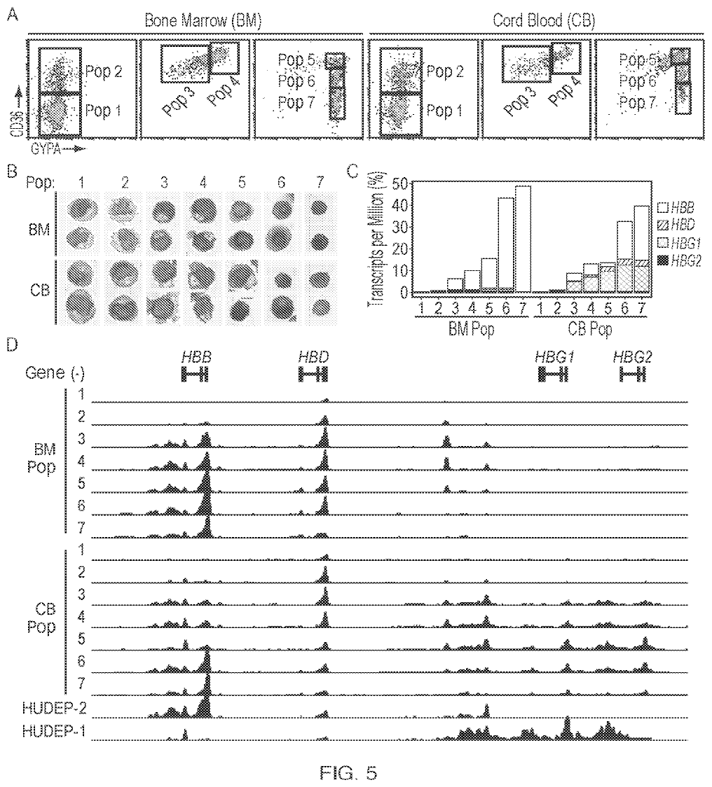

Using fluorescence-activated cell sorting (FACS), seven increasingly mature, stage-matched cell populations of fetal and adult state RBCs were isolated based on expression of erythroid surface proteins. Previous studies concerning the regulatory networks driving the fetal and adult states of hemoglobin have relied on asynchronous pooled erythroid cells at different stages of differentiation, which can confound the identification of lineage- and stage-specific regulators of fetal hemoglobin. Others have used sorted bone marrow cells, and more recently, performed comparative RNA-seq profiling of FACS sorted adult and neonatal cell populations. The sorting approach was built upon to include chromatin accessibility profiling of the stage-matched, sorted cells. Using the well-established three-phase erythroid differentiation system described by Giarratana (Blood 2011), BM- and CB-derived CD34+ cells were cultured and sorted to seven discrete BM and CB cell populations based on expression of two well-characterized erythroid surface markers (CD36 and Glycophorin A) (FIG. 5A). The purity and differentiation state of the sorted cell populations were confirmed by flow cytometry and, visually, by performing cytospin analysis. Representative images of the seven increasingly mature populations of cells were obtained and it was found that, with increasing maturity, both BM and CB cells decreased in size and hemoglobinization, and an increase in the ratio of the nucleus:cytoplasm was observed (FIG. 5B). With increasing maturity, each population from the BM and CB expressed increasing levels of total beta-globin like mRNA, with the majority of transcripts in BM-derived cells consisting of HBB transcripts and the majority of transcripts in CB-derived cells produced from HBG (FIG. 5C).

BM and CB cells exhibit distinct patterns of chromatin accessibility at the HBB locus: Next, the accessible chromatin at the HBB locus in the BM- and CB-derived cell populations was compared using ATAC-seq (FIG. 5D). Briefly, ATAC-seq is a highly sensitive technique that relies on the property of the transposase enzyme to integrate into active regulatory elements. It uses a hyperactive Tn5 transposase to integrate its adapter payload into regions of open or accessible chromatin-accessible chromatin sites of the human genome. The integrated adapter enables high throughput sequencing to identify regions of open (or active) chromatin. The resulting footprint sequence can be visualized in a bioinformatics software to reveal the presence or absence of DNA-binding proteins genome-wide.

In both BM- and CB-derived cells, chromatin accessibility is initiated at the HBD locus, as observed in the populations designated 2-4. In the BM-derived cells, increasing chromatin accessibility at the HBB promoter was observed throughout erythroid differentiation. In CB-derived cells, increased chromatin accessibility was observed at the HBG promoter in populations 1-5, and a reversion back to the HBB promoter in populations 6 and 7 was also observed, suggesting that HBG gene expression is transient in CB-derived erythroid cells. Immortalized erythroid cell lines, HUDEP-1 and HUDEP-2, which express fetal and adult hemoglobin respectively, were also included as controls for the ATAC-seq experiments and expected chromatin accessibility profiles were observed. In sum, accessibility of the chromatin was initiated at the HBD promoter; was greater at the HBB promoter in BM-derived cells; and was greater in the HBG promoter in CB-derived cells. HBG gene expression appears to be transient in CB-derived cells.

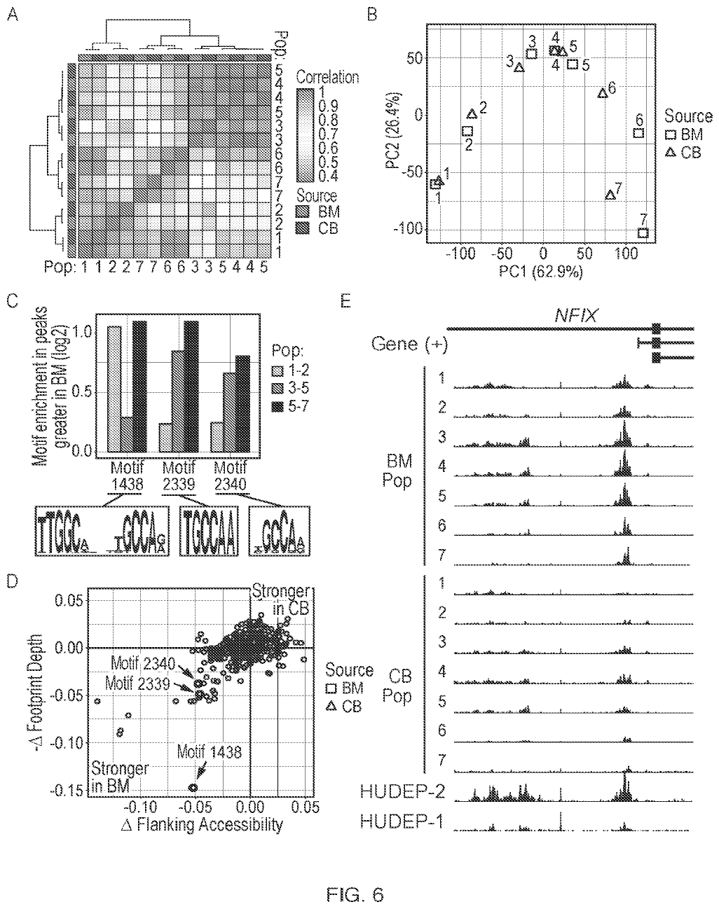

The majority of molecular changes during erythroid maturation are stage-specific: Next, hierarchical clustering and principal component analysis (PCA) of the RNA-seq and ATAC-seq data derived from sorted BM and CB cell populations was performed (FIGS. 6A and 6B). The hierarchical clustering and PCA on RNA-seq data showed that sorted populations clustered based on their differentiation state. Similarly, the hierarchical clustering and PCA on ATAC-seq peaks clustered the BM and CB cell populations together. This suggests the majority of molecular changes during erythroid differentiation are not specific to BM or CB lineages, but rather depend on the differentiation state of the cells.

Flanking accessibility and footprint depth at NF1 motifs were also increased in BM cells: To identify transcription factors driving the cell state, and potentially beta-like globin expression preference, a comprehensive analysis of DNA binding motifs within regions of differential chromatin accessibility was performed. Three consensus sequence motifs were identified from the NFI family of transcription factors that were enriched in ATAC-seq peaks that were larger in BM-derived cells relative to CB-derived cells (FIG. 6C). This observation is consistent with findings from Lessard et al. (Genome Med, 2015), which reported enrichment of NFI motifs in adult erythroblasts compared to fetal-state erythroblasts. Enrichment of these motifs in BM cells was confirmed by transcriptional footprint analysis (Corces et al., Science, 2018) that considers the depth of the ATAC-seq peaks (footprint depth) and their flanking accessibility, which together indicate the relative strength of transcription factor binding (FIG. 6D)

In BM cells, the NFIX promoter had increased chromatin accessibility: To advance the study, the chromatin accessibility in BM- and CB-derived cells at the promoters of four closely-related NF1 family transcription factors was compared: NFIA, B, C, and X. These transcription factors have a role in activation and repression of several target genes in various organs. Once again, HUDEP-1 and HUDEP-2 cells were included as controls. At the NFIX promoter, increased chromatin accessibility was observed in all BM-derived cells throughout differentiation relative to the CB-derived cells (FIG. 6E). These data suggest that NFIX is a putative fetal hemoglobin repressor in adult erythroid cells. Other reports in the literature have also implicated a role for NFIX in fetal hemoglobin regulation. A genome-wide association analysis (GWAS) study published by Danjou et al. (Nat. Genet. 47(11):1264-1271, 2015) identified a SNP just below statistical significance in an NFIX intron, which correlates with higher levels of HbF. The reported SNP is approximately 10 kb upstream of the NFIX differential chromatin accessibility region and is in close proximity to hypomethylated CpGs in adult erythroblasts reported by Lessard et al. (supra). The data generated, particularly when considered in light of these published reports, strongly suggest that NFIX functions as an HbF repressor, and the work described below validates its role in fetal hemoglobin regulation.

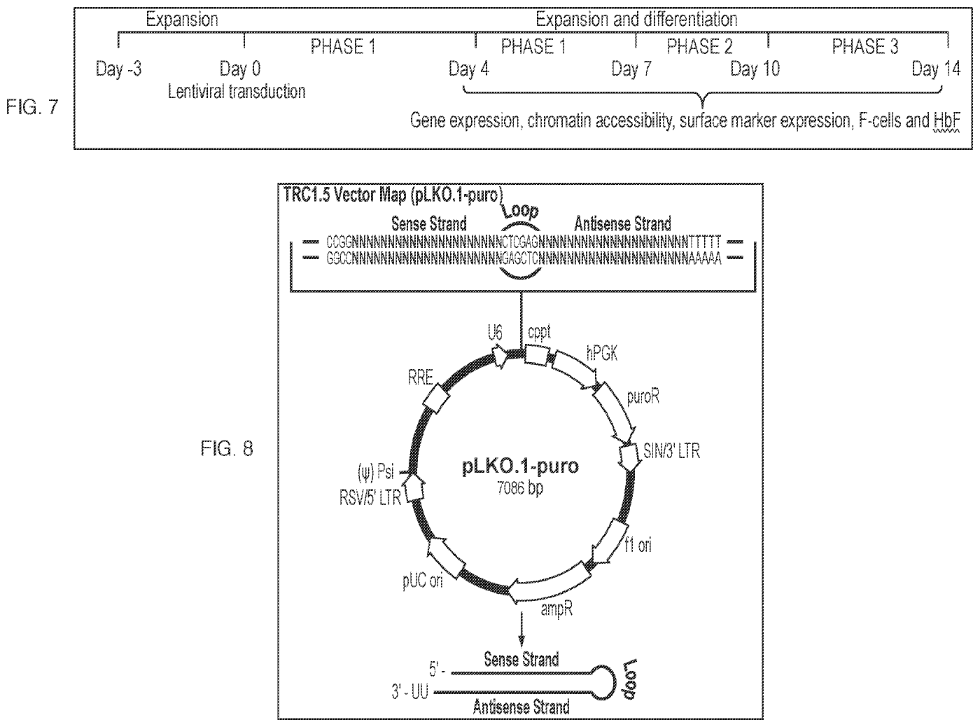

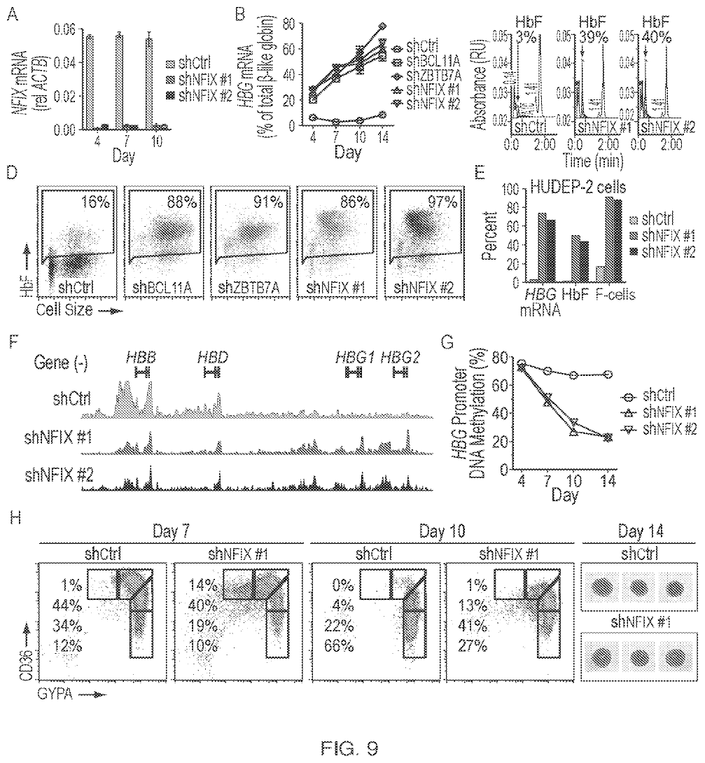

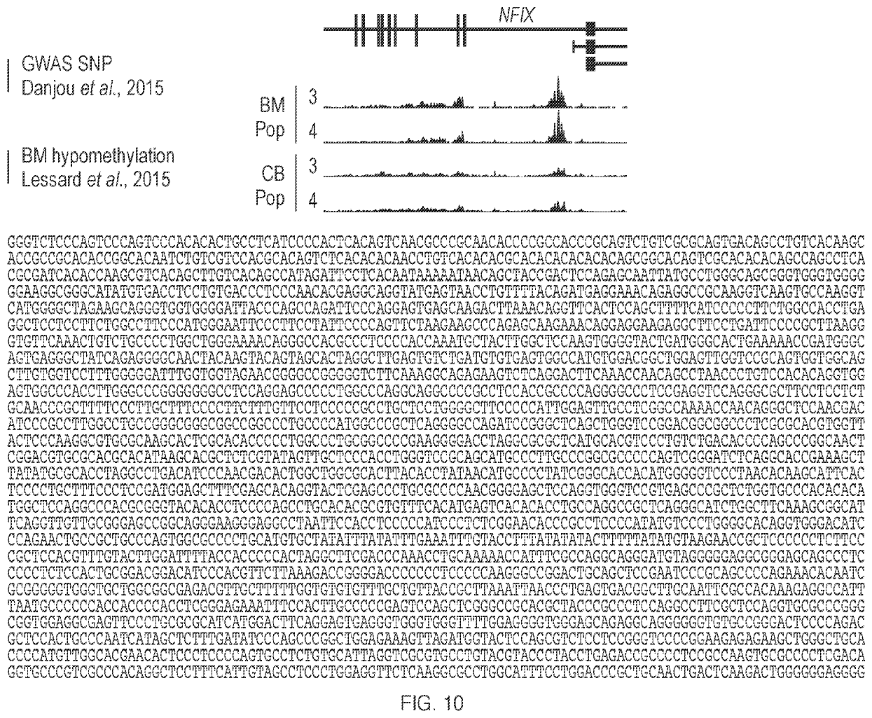

RNAi leads to robust knockdown of NFIX in BM-derived cells: To generate NFIX knockdowns in primary BM cells, lentiviral-mediated transduction was used to deliver five different short hairpin RNAs (shRNAs) on Day 0 of the three-phase erythroid culture system (see FIG. 7). Cells were harvested at different time points during culture to assess gene expression, chromatin accessibility, erythroid maturation and fetal globin induction. Knockdown of NFIX was confirmed by qPCR (FIG. 9A) and Western blot (not shown) and a >90% reduction of NFIX was observed at the transcript level and about a 70% reduction at the protein level. NFIX knockdown was achieved using two different shRNAs obtained from Sigma:

(SEQ ID NO:_)
CCGGACATTGGAGTCACAATCAAAGCTCGAGCTTTGATTG
TGACTCCAATGTTTTTTG
and
(SEQ ID NO:_)
CCGGGGAATCCGGACAATCAGATAGCTCGAGCTATCTGAT
TGTCCGGATTCCTTTTTG.

The shRNAs were delivered in the TRC1.5 vector construct shown in FIG. 8.

NFIX knockdown in primary BM cells leads to elevated HBG mRNA and HbF protein levels: The effect of NFIX knockdown on HBG gene expression was also assessed. It was found that >90% knockdown of NFIX transcripts, which was confirmed by Western blot, led to a dramatic time-dependent increase in HBG transcripts, to a level comparable to known HbF repressors, BCL11A and ZBTB7A (FIG. 9B and FIG. 9D). An increase in HBG transcripts correlated with an increase in HbF protein (FIG. 9C). The percentages of F-cells in NFIX knockdown cells were measured using a fluorescently-tagged antibody targeting HbF. An F-cell is a cell that contains a detectable level of HbF by flow cytometry. As can be seen in the middle panel of FIG. 9D, knockdown of NFIX leads to a 5-6-fold increase in the percentage of cells that are positive for fetal hemoglobin, as well as an increase in the amount of HbF per cell. The relative levels of HbF correlated with absolute HbF levels in the NFIX knocked down cells as measured by HPLC and were greater than the clinical curative benchmark of 30% HbF, a level observed in individuals with hereditary persistence of fetal hemoglobin (FIGS. 9C and 9D). Additionally, the HBG mRNA and HbF protein level findings were replicated in HUDEP-2 cells, further strengthening the validation of NFIX as a bona fide HbF repressor (FIG. 9E).

NFIX knockdown leads to functional changes at the HBG promoter: When chromatin accessibility at the HBG promoter was examined, it was found that NFIX knockdown leads to a reduction in chromatin accessibility at the HBB locus and an increase in chromatin accessibility at the HBG locus, representing an adult-to-fetal hemoglobin switch (FIG. 9F). DNA methylation at several CpGs upstream of the HBG1 and HBG2 transcription start sites was assessed. DNA methylation of one of the CpGs is shown in FIG. 9G and, as can be seen, NFIX knockdown leads to a decrease in DNA methylation at the HBG promoter. This suggests de-repression of the HBG locus. The reduction in CpG methylation was also observed for 5 other CpGs at the HBG promoter as well.

NFIX-knockdown cells are capable of terminal erythroid differentiation: During each phase of the erythroid culture described above, the impact of NFIX knockdown on erythroid differentiation was also assessed. A delay in erythroid maturation on Days 7 and 10 of erythroid culture was observed as represented by the surface marker expression profiles of the control and KD cells (FIG. 9H). However, by Day 14 the NFIX-knocked down cells matured to produce enucleated hemoglobinized reticulocytes, suggesting that these genetically modified cells are capable of terminal erythroid differentiation.

Taken together, the studies described here indicate that NFIX is a fetal hemoglobin repressor. Using FACS, discrete BM and CB cell populations were sorted, which were subjected to whole transcriptome profiling and ATAC-seq. Chromatin accessibility analysis identified motifs for NFI family transcription factors to be enriched under ATAC-seq peaks that were larger in BM cells relative to CB populations. A region of increased chromatin accessibility at the NFIX promoter was observed in BM cells compared to the CB cells. RNAi KD of NFIX in both primary BM cells and HUDEP-2 cells led to a time-dependent induction of gamma globin mRNA and HbF protein, thereby validating the role of NFIX as an HbF repressor.

1. A method of treating a hemoglobinopathy in a patient in need thereof, the method comprising administering to the patient an effective amount of a pharmaceutically acceptable composition comprising a genetically modified cell, wherein the cell comprises a nucleic acid construct that inhibits the expression of NFIX within the cell.

2. The method of claim 1, wherein the hemoglobinopathy is a β-thalassemia.

3. The method of claim 1, wherein the hemoglobinopathy is sickle cell disease (SCD).

4. The method of claim 1, wherein the cell is a hematopoietic stem cell, a hematopoietic progenitor cell, or an erythroblast at any stage of development prior to enucleation.

5. The method of claim 4, wherein the cell is autologous to the patient.

6. The method of claim 1, wherein the nucleic acid construct comprises an shRNA or antisense nucleic acid sequence that inhibits the expression of NFIX

7. The method of claim 6, wherein the shRNA or antisense nucleic acid sequence base pairs with a sequence within the region of NFIX that encodes a DNA-binding domain or within the NFIX promoter/regulatory region (SEQ ID NO:761).

8. The method of claim 7, wherein the DNA-binding domain has the sequence
(SEQ ID [[NO:_]]NO: 1)
KQKWASRLLAKLRKDIRPEFREDFVLTITGKKPPCCVLSNPDQKGKIRR
IDCLRQADKVWRLDLVMVILFKGIPLESTDGERLYKSPQCSNPGLCVQP
HHIGV.

9. The method of claim 6, wherein the shRNA or antisense nucleic acid sequence base pairs with a sequence within Exon 2 and/or Exon 3 of NFIX.

10. The method of claim 1, wherein the nucleic acid construct comprises an shRNA comprising a sequence selected from the group consisting of:
(SEQ ID [[NO: ]]259)
CCGGGCAGTCTCAGTCCTGGTTCCTCTCGAGAGGAACCAGGACTGAGAC
TGCTTTTT;
(SEQ ID [[NO: ]]261)
CCGGACTGGATCTTTATCTGGCTTACTCGAGTAAGCCAGATAAAGATCC
AGTTTTTT;
(SEQ ID [[NO: ]]760)
CCGGCCGGCTTCTCTAAAGAAGTCACTCGAGTGACTTCTTTAGAGAAGC
CGGTTTTT;
(SEQ ID [[NO: ]]257)
CCGGCACATCACATTGGAGTCACAACTCGAGTTGTGACTCCAATGTGAT
GTGTTTTT;
(SEQ ID [[NO: ]]256)
CCGGCCTGTTGATGACGTGTTCTATCTCGAGATAGAACACGTCATCAAC
AGGTTTTT;
(SEQ ID [[NO: ]]262)
CCGGATCTTTATCTGGCTTACTTTGCTCGAGCAAAGTAAGCCAGATAAA
GATTTTTTG;
(SEQ ID [[NO:_]]2)
CCGGACATTGGAGTCACAATCAAAGCTCGAGCTTTGATTGTGACTCCAA
TGTTTTTTG;
(SEQ ID [[NO:_]]3)
CCGGGGAATCCGGACAATCAGATAGCTCGAGCTATCTGATTGTCCGGAT
TCCTTTTTG;
(SEQ ID [[NO:_]]263)
CCGGTAGGCTAAGAAACGCAGTATACTCGAGTATACTGCGTTTCTTAGC
CTATTTTTG;
and
(SEQ ID [[NO: ]]260)
CCGGCCCAACGGGCACTTAAGTTTCCTCGAGGAAACTTAAGTGCCCGTT
GGGTTTTTG.
or a sequence at least 90% identical thereto, optionally wherein the shRNA terminates at the residue immediately prior to the terminal poly-T sequence or comprises only the internally complementary sequences.

11. The method of claim 10, wherein the nucleic acid construct comprises an shRNA comprising
(SEQ ID [[NO:_]]2)
CCGGACATTGGAGTCACAATCAAAGCTCGAGCTTTGATTGTGACTCCAA
TGTTTTTTG
or
(SEQ ID [[NO:_]]3)
CCGGGGAATCCGGACAATCAGATAGCTCGAGCTATCTGATTGTCCGGAT
TCCTTTTTG.

12. The method of claim 1, wherein the genetically modified cell comprises the components of a nuclease-editing system.

13. The method of claim 12, wherein the nuclease-editing system is CRISPR or the genetically modified cell comprises a transposon.

14. The method of claim 13, wherein the genetically modified cell comprises a guide RNA that base pairs with a sequence within the region of NFIX that encodes a DNA-binding domain or within the NFIX promoter/regulatory region (SEQ ID NO:761).

15. A non-naturally occurring, genetically modified cell, wherein the level of NFIX expression in the cell is decreased relative to that of a comparable, non-genetically modified cell of the same type.

16. The cell of claim 15, wherein the cell is a hematopoietic stem cell, a hematopoietic progenitor cell, or an erythroblast at any stage of development prior to enucleation.

17. The cell of claim 15, wherein the cell comprises an shRNA or antisense nucleic acid sequence that inhibits the expression of NFIX.

18. A pharmaceutically acceptable composition comprising the cell of claim 15.

19. The pharmaceutically acceptable composition of claim 18, formulated for intravenous administration.

20. A kit comprising the cell of claim 15, optionally within a pharmaceutically acceptable composition, and instructions for use.


SEQUENCE LISTING (764 sequences)

SEQ ID NO: 1 - protein, 103 aa, Homo sapiens
KQKWASRLLAKLRKDIRPEFREDFVLTITGKKPPCCVLSNPDQKGKIRRIDCLRQADKVWRLDLVMVILFKGIPLESTDGERLYKSPQCSNPGLCVQPHHIGV

SEQ ID NO: 2 - DNA, 58 bp, Artificial Sequence
  Description of Artificial Sequence Synthetic oligonucleotide
ccggacattggagtcacaatcaaagctcgagctttgattgtgactccaatgttttttg

SEQ ID NO: 3 - DNA, 58 bp, Artificial Sequence
  Description of Artificial Sequence Synthetic oligonucleotide
ccggggaatccggacaatcagatagctcgagctatctgattgtccggattcctttttg

SEQ ID NO: 4 - protein, 510 aa, Homo sapiens
MEMSWARQSLAGIPAACDEFHPFIEALLPHVRAFSYTWFNLQARKRKYFKKHEKRMSKDEERAVKDELLGEKPEIKQKWASRLLAKLRKDIRPEFREDFVLTITGKKPPCCVLSNPDQKGKIRRIDCLRQADKVWRLDLVMVILFKGIPLESTDGERLYKSPQCSNPGLCVQPHHIGVTIKELDLYLAYFVHTPESGQSDSSNQQGDADIKPLPNGHLSFQDCFVTSGVWNVTELVRVSQTPVATASGPNFSLADLESPSYYNINQVTLGRRSITSPPSTSTTKRPKSIDDSEMESPVDDVFYPGTGRSPAAGSSQSSGWPNDVDAGPASLKKSGKLDFCSALSSQGSSPRMAFTHHPLPVLAGVRPGSPRATASALHFPSTSIIQQSSPYFTHPTIRYHHHHGQDSLKEFVQFVCSDGSGQATGQPNGSGQGKVPGSFLLPPPPPVARPVPLPMPDSKSTSTAPDGAALTPPSPSFATTGASSANRFVSIGPRDGNFLNIPQQSQSWFL

SEQ ID NO: 5 - DNA, 1533 bp, Homo sapiens
atggagatgtcatgggcgcgacagagcctggcggggataccagcagcgtgtgatgagttccacccgttcatcgaggcactgctgcctcacgtccgcgctttctcctacacctggttcaacctgcaggcgcggaagcgcaagtacttcaagaagcatgaaaagcggatgtcgaaggacgaggagcgggcggtgaaggacgagctgctgggcgagaagcccgagatcaagcagaagtgggcatcccggctgctggccaagctgcgcaaggacatccggcccgagttccgcgaggacttcgtgctgaccatcacgggcaagaagcccccctgctgcgtgctctccaaccccgaccagaagggcaagatccggcggattgactgcctgcgccaggctgacaaggtgtggcggctggacctggtcatggtgattttgtttaaggggatccccctggaaagtactgatggggagcggctctacaagtcgcctcagtgctcgaaccccggcctgtgcgtccagccacatcacattggagtcacaatcaaagaactggatctttatctggcttactttgtccacactccggaatccggacaatcagatagttcaaaccagcaaggagatgcggacatcaaaccactgcccaacgggcacttaagtttccaggactgttttgtgacttccggggtctggaatgtgacggagctggtgagagtatcacagactcctgttgcaacagcatcagggcccaacttctccctggcggacctggagagtcccagctactacaacatcaaccaggtgaccctggggcggcggtccatcacctcccctccttccaccagcaccaccaagcgccccaagtccatcgatgacagtgagatggagagccctgttgatgacgtgttctatcccgggacaggccgttccccagcagctggcagcagccagtccagcgggtggcccaacgatgtggatgcaggcccggcttctctaaagaagtcaggaaagctggacttctgcagtgccctctcctctcagggcagctccccgcgcatggctttcacccaccacccgctgcctgtgcttgctggagtcagaccagggagcccccgggccacagcatcagccctgcacttcccctccacgtccatcatccagcagtcgagcccgtatttcacgcacccgaccatccgctaccaccaccaccacgggcaggactcactgaaggagtttgtgcagtttgtgtgctcggatggctcgggccaggccaccggacagcccaacggtagcggccagggcaaagtcccggggtcatttttgctaccgccgccgcctccagtggccagacctgtgccccttcctatgcctgattccaaatccaccagcactgccccagacggcgccgccttgactcctccatcaccttcattcgcaacgacaggcgcctcctctgccaaccggtttgtcagcatcggaccccgggacggcaactttctgaacatcccacagcagtctcagtcctggttcctctga

SEQ ID NO: 6 - protein, 441 aa, Homo sapiens
MYSPYCLTQDEFHPFIEALLPHVRAFSYTWFNLQARKRKYFKKHEKRMSKDEERAVKDELLGEKPEIKQKWASRLLAKLRKDIRPEFREDFVLTITGKKPPCCVLSNPDQKGKIRRIDCLRQADKVWRLDLVMVILFKGIPLESTDGERLYKSPQCSNPGLCVQPHHIGVTIKELDLYLAYFVHTPESGQSDSSNQQGDADIKPLPNGHLSFQDCFVTSGVWNVTELVRVSQTPVATASGPNFSLADLESPSYYNINQVTLGRRSITSPPSTSTTKRPKSIDDSEMESPVDDVFYPGTGRSPAAGSSQSSGWPNDVDAGPASLKKSGKLDFCSALSSQGSSPRMAFTHHPLPVLAGVRPGSPRATASALHFPSTSIIQQSSPYFTHPTIRYHHHHGQDSLKEFVQFVCSDGSGQATGQHSQRQAPPLPTGLSASDPGTATF

SEQ ID NO: 7 - DNA, 1326 bp, Homo sapiens
atgtactccccgtactgcctcacccaggatgagttccacccgttcatcgaggcactgctgcctcacgtccgcgctttctcctacacctggttcaacctgcaggcgcggaagcgcaagtacttcaagaagcatgaaaagcggatgtcgaaggacgaggagcgggcggtgaaggacgagctgctgggcgagaagcccgagatcaagcagaagtgggcatcccggctgctggccaagctgcgcaaggacatccggcccgagttccgcgaggacttcgtgctgaccatcacgggcaagaagcccccctgctgcgtgctctccaaccccgaccagaagggcaagatccggcggattgactgcctgcgccaggctgacaaggtgtggcggctggacctggtcatggtgattttgtttaaggggatccccctggaaagtactgatggggagcggctctacaagtcgcctcagtgctcgaaccccggcctgtgcgtccagccacatcacattggagtcacaatcaaagaactggatctttatctggcttactttgtccacactccggaatccggacaatcagatagttcaaaccagcaaggagatgcggacatcaaaccactgcccaacgggcacttaagtttccaggactgttttgtgacttccggggtctggaatgtgacggagctggtgagagtatcacagactcctgttgcaacagcatcagggcccaacttctccctggcggacctggagagtcccagctactacaacatcaaccaggtgaccctggggcggcggtccatcacctcccctccttccaccagcaccaccaagcgccccaagtccatcgatgacagtgagatggagagccctgttgatgacgtgttctatcccgggacaggccgttccccagcagctggcagcagccagtccagcgggtggcccaacgatgtggatgcaggcccggcttctctaaagaagtcaggaaagctggacttctgcagtgccctctcctctcagggcagctccccgcgcatggctttcacccaccacccgctgcctgtgcttgctggagtcagaccagggagcccccgggccacagcatcagccctgcacttcccctccacgtccatcatccagcagtcgagcccgtatttcacgcacccgaccatccgctaccaccaccaccacgggcaggactcactgaaggagtttgtgcagtttgtgtgctcggatggctcgggccaggccaccggacagcattcgcaacgacaggcgcctcctctgccaaccggtttgtcagcatcggaccccgggacggcaactttctga

SEQ ID NO: 8 - protein, 433 aa, Homo sapiens
MDEFHPFIEALLPHVRAFSYTWFNLQARKRKYFKKHEKRMSKDEERAVKDELLGEKPEIKQKWASRLLAKLRKDIRPEFREDFVLTITGKKPPCCVLSNPDQKGKIRRIDCLRQADKVWRLDLVMVILFKGIPLESTDGERLYKSPQCSNPGLCVQPHHIGVTIKELDLYLAYFVHTPESGQSDSSNQQGDADIKPLPNGHLSFQDCFVTSGVWNVTELVRVSQTPVATASGPNFSLADLESPSYYNINQVTLGRRSITSPPSTSTTKRPKSIDDSEMESPVDDVFYPGTGRSPAAGSSQSSGWPNDVDAGPASLKKSGKLDFCSALSSQGSSPRMAFTHHPLPVLAGVRPGSPRATASALHFPSTSIIQQSSPYFTHPTIRYHHHHGQDSLKEFVQFVCSDGSGQATGQHSQRQAPPLPTGLSASDPGTATF

SEQ ID NO: 9 - DNA, 1302 bp, Homo sapiens
atggatgagttccacccgttcatcgaggcactgctgcctcacgtccgcgctttctcctacacctggttcaacctgcaggcgcggaagcgcaagtacttcaagaagcatgaaaagcggatgtcgaaggacgaggagcgggcggtgaaggacgagctgctgggcgagaagcccgagatcaagcagaagtgggcatcccggctgctggccaagctgcgcaaggacatccggcccgagttccgcgaggacttcgtgctgaccatcacgggcaagaagcccccctgctgcgtgctctccaaccccgaccagaagggcaagatccggcggattgactgcctgcgccaggctgacaaggtgtggcggctggacctggtcatggtgattttgtttaaggggatccccctggaaagtactgatggggagcggctctacaagtcgcctcagtgctcgaaccccggcctgtgcgtccagccacatcacattggagtcacaatcaaagaactggatctttatctggcttactttgtccacactccggaatccggacaatcagatagttcaaaccagcaaggagatgcggacatcaaaccactgcccaacgggcacttaagtttccaggactgttttgtgacttccggggtctggaatgtgacggagctggtgagagtatcacagactcctgttgcaacagcatcagggcccaacttctccctggcggacctggagagtcccagctactacaacatcaaccaggtgaccctggggcggcggtccatcacctcccctccttccaccagcaccaccaagcgccccaagtccatcgatgacagtgagatggagagccctgttgatgacgtgttctatcccgggacaggccgttccccagcagctggcagcagccagtccagcgggtggcccaacgatgtggatgcaggcccggcttctctaaagaagtcaggaaagctggacttctgcagtgccctctcctctcagggcagctccccgcgcatggctttcacccaccacccgctgcctgtgcttgctggagtcagaccagggagcccccgggccacagcatcagccctgcacttcccctccacgtccatcatccagcagtcgagcccgtatttcacgcacccgaccatccgctaccaccaccaccacgggcaggactcactgaaggagtttgtgcagtttgtgtgctcggatggctcgggccaggccaccggacagcattcgcaacgacaggcgcctcctctgccaaccggtttgtcagcatcggaccccgggacggcaactttctga

SEQ ID NO: 10 - DNA, 21 bp, Homo sapiens
cctgttgatgacgtgttctat

SEQ ID NO: 11 - DNA, 21 bp, Homo sapiens
cacatcacattggagtcacaa

SEQ ID NO: 12 - DNA, 21 bp, Homo sapiens
ccggacaatcagatagttcaa

SEQ ID NO: 13 - DNA, 21 bp, Homo sapiens
ggaatccggacaatcagatag

SEQ ID NO: 14 - DNA, 21 bp, Homo sapiens
cccaacgggcacttaagtttc

SEQ ID NO: 15 - DNA, 21 bp, Homo sapiens
acattggagtcacaatcaaag

SEQ ID NO: 16 - DNA, 21 bp, Homo sapiens
atctttatctggcttactttg

SEQ ID NO: 17 - DNA, 21 bp, Homo sapiens
taggctaagaaacgcagtata

SEQ ID NO: 18 - DNA, 21 bp, Homo sapiens
tggacctggtcatggtgattt

SEQ ID NO: 19 - DNA, 21 bp, Homo sapiens
ggatgagttccacccgttcat

SEQ ID NO: 20 - DNA, 21 bp, Homo sapiens
tttctcctacacctggttcaa

SEQ ID NO: 21 - DNA, 21 bp, Homo sapiens
ttctcctacacctggttcaac

SEQ ID NO: 22 - DNA, 21 bp, Homo sapiens
gcggaagcgcaagtacttcaa

SEQ ID NO: 23 - DNA, 21 bp, Homo sapiens
cggaagcgcaagtacttcaag

SEQ ID NO: 24 - DNA, 21 bp, Homo sapiens
ggaagcgcaagtacttcaaga

SEQ ID NO: 25 - DNA, 21 bp, Homo sapiens
gaagcgcaagtacttcaagaa

SEQ ID NO: 26 - DNA, 21 bp, Homo sapiens
agcccgagatcaagcagaagt

SEQ ID NO: 27 - DNA, 21 bp, Homo sapiens
ctggacctggtcatggtgatt

SEQ ID NO: 28 - DNA, 21 bp, Homo sapiens
tgcgtccagccacatcacatt

SEQ ID NO: 29 - DNA, 21 bp, Homo sapiens
acatcacattggagtcacaat

SEQ ID NO: 30 - DNA, 21 bp, Homo sapiens
atcacattggagtcacaatca

SEQ ID NO: 31 - DNA, 21 bp, Homo sapiens
cattggagtcacaatcaaaga

SEQ ID NO: 32 - DNA, 21 bp, Homo sapiens
ttggagtcacaatcaaagaac

SEQ ID NO: 33 - DNA, 21 bp, Homo sapiens
tggagtcacaatcaaagaact

SEQ ID NO: 34 - DNA, 21 bp, Homo sapiens
acaatcaaagaactggatctt

SEQ ID NO: 35 - DNA, 21 bp, Homo sapiens
tggatctttatctggcttact

SEQ ID NO: 36 - DNA, 21 bp, Homo sapiens
tctttatctggcttactttgt

SEQ ID NO: 37 - DNA, 21 bp, Homo sapiens
ctttatctggcttactttgtc

SEQ ID NO: 38 - DNA, 21 bp, Homo sapiens
acactccggaatccggacaat

SEQ ID NO: 39 - DNA, 21 bp, Homo sapiens
actccggaatccggacaatca

SEQ ID NO: 40 - DNA, 21 bp, Homo sapiens
ccggaatccggacaatcagat

SEQ ID NO: 41 - DNA, 21 bp, Homo sapiens
cggaatccggacaatcagata

SEQ ID NO: 42 - DNA, 21 bp, Homo sapiens
gaatccggacaatcagatagt

SEQ ID NO: 43 - DNA, 21 bp, Homo sapiens
atccggacaatcagatagttc

SEQ ID NO: 44 - DNA, 21 bp, Homo sapiens
tccggacaatcagatagttca

SEQ ID NO: 45 - DNA, 21 bp, Homo sapiens
agcaaggagatgcggacatca

SEQ ID NO: 46 - DNA, 21 bp, Homo sapiens
gcaaggagatgcggacatcaa

SEQ ID NO: 47 - DNA, 21 bp, Homo sapiens
acttaagtttccaggactgtt

SEQ ID NO: 48 - DNA, 21 bp, Homo sapiens
cttaagtttccaggactgttt

SEQ ID NO: 49 - DNA, 21 bp, Homo sapiens
tgacggagctggtgagagtat

SEQ ID NO: 50 - DNA, 21 bp, Homo sapiens
acggagctggtgagagtatca

SEQ ID NO: 51 - DNA, 21 bp, Homo sapiens
tggagagtcccagctactaca

SEQ ID NO: 52 - DNA, 21 bp, Homo sapiens
ggagagtcccagctactacaa

SEQ ID NO: 53 - DNA, 21 bp, Homo sapiens
gagagtcccagctactacaac

SEQ ID NO: 54 - DNA, 21 bp, Homo sapiens
agagtcccagctactacaaca

SEQ ID NO: 55 - DNA, 21 bp, Homo sapiens
gagtcccagctactacaacat

SEQ ID NO: 56 - DNA, 21 bp, Homo sapiens
agtcccagctactacaacatc

SEQ ID NO: 57 - DNA, 21 bp, Homo sapiens
gtcccagctactacaacatca

SEQ ID NO: 58 - DNA, 21 bp, Homo sapiens
tcccagctactacaacatcaa

SEQ ID NO: 59 - DNA, 21 bp, Homo sapiens
cccagctactacaacatcaac

SEQ ID NO: 60 - DNA, 21 bp, Homo sapiens
gtccatcgatgacagtgagat

SEQ ID NO: 61 - DNA, 21 bp, Homo sapiens
tgagatggagagccctgttga

SEQ ID NO: 62 - DNA, 21 bp, Homo sapiens
gagatggagagccctgttgat

SEQ ID NO: 63 - DNA, 21 bp, Homo sapiens
agatggagagccctgttgatg

SEQ ID NO: 64 - DNA, 21 bp, Homo sapiens
gatggagagccctgttgatga

SEQ ID NO: 65 - DNA, 21 bp, Homo sapiens
gccctgttgatgacgtgttct

SEQ ID NO: 66 - DNA, 21 bp, Homo sapiens
ccctgttgatgacgtgttcta

SEQ ID NO: 67 - DNA, 21 bp, Homo sapiens
ttctctaaagaagtcaggaaa

SEQ ID NO: 68 - DNA, 21 bp, Homo sapiens
agtcaggaaagctggacttct

SEQ ID NO: 69 - DNA, 21 bp, Homo sapiens
caggactcactgaaggagttt

SEQ ID NO: 70 - DNA, 21 bp, Homo sapiens
ggactcactgaaggagtttgt

SEQ ID NO: 71 - DNA, 21 bp, Homo sapiens
gactcactgaaggagtttgtg

SEQ ID NO: 72 - DNA, 21 bp, Homo sapiens
ctgaaggagtttgtgcagttt

SEQ ID NO: 73 - DNA, 21 bp, Homo sapiens
gaaggagtttgtgcagtttgt

SEQ ID NO: 74 - DNA, 21 bp, Homo sapiens
cttcctatgcctgattccaaa

SEQ ID NO: 75 - DNA, 21 bp, Homo sapiens
cctatgcctgattccaaatcc

SEQ ID NO: 76 - DNA, 21 bp, Homo sapiens
tgactcctccatcaccttcat

SEQ ID NO: 77 - DNA, 21 bp, Homo sapiens
gggacggcaactttctgaaca

SEQ ID NO: 78 - DNA, 21 bp, Homo sapiens
ggacggcaactttctgaacat

SEQ ID NO: 79 - DNA, 21 bp, Homo sapiens
gacggcaactttctgaacatc

SEQ ID NO: 80 - DNA, 21 bp, Homo sapiens
ctcagtcctggttcctctgat

SEQ ID NO: 81 - DNA, 21 bp, Homo sapiens
tcagtcctggttcctctgata

SEQ ID NO: 82 - DNA, 21 bp, Homo sapiens
tcctctgataagatcgacaaa

SEQ ID NO: 83 - DNA, 21 bp, Homo sapiens
agaagaagaggttcctcgaaa

SEQ ID NO: 84 - DNA, 21 bp, Homo sapiens
accagcagccaagcagaaaga

SEQ ID NO: 85 - DNA, 21 bp, Homo sapiens
gcagccaagcagaaagaaaca

SEQ ID NO: 86 - DNA, 21 bp, Homo sapiens
cagccaagcagaaagaaacac

SEQ ID NO: 87 - DNA, 21 bp, Homo sapiens
acgcgacatggactctgtcaa

SEQ ID NO: 88 - DNA, 21 bp, Homo sapiens
gcgacatggactctgtcaagt

SEQ ID NO: 89 - DNA, 21 bp, Homo sapiens
cgacatggactctgtcaagta

SEQ ID NO: 90 - DNA, 21 bp, Homo sapiens
gacatggactctgtcaagtag

SEQ ID NO: 91 - DNA, 21 bp, Homo sapiens
acatggactctgtcaagtaga

SEQ ID NO: 92 - DNA, 21 bp, Homo sapiens
gtagaggacagaaagcaagaa

SEQ ID NO: 93 - DNA, 21 bp, Homo sapiens
agcaagaaaggatgcagaact

SEQ ID NO: 94 - DNA, 21 bp, Homo sapiens
agcaaccagcacaattctgaa

SEQ ID NO: 95 - DNA, 21 bp, Homo sapiens
gagggccagcttgtaaagatg

SEQ ID NO: 96 - DNA, 21 bp, Homo sapiens
agggccagcttgtaaagatga

SEQ ID NO: 97 - DNA, 21 bp, Homo sapiens
gggccagcttgtaaagatgat

SEQ ID NO: 98 - DNA, 21 bp, Homo sapiens
ggccagcttgtaaagatgatg

SEQ ID NO: 99 - DNA, 21 bp, Homo sapiens
gctagttgcgtgtcccagaat

SEQ ID NO: 100 - DNA, 21 bp, Homo sapiens
tcccagaatggagggtgttct

SEQ ID NO: 101 - DNA, 21 bp, Homo sapiens
ctctgtacagtccgtaggaaa

SEQ ID NO: 102 - DNA, 21 bp, Homo sapiens
agtacttcaagaagcatgaaa

SEQ ID NO: 103 - DNA, 21 bp, Homo sapiens
tcacattggagtcacaatcaa

SEQ ID NO: 104 - DNA, 21 bp, Homo sapiens
cacattggagtcacaatcaaa

SEQ ID NO: 105 - DNA, 21 bp, Homo sapiens
attggagtcacaatcaaagaa

SEQ ID NO: 106 - DNA, 21 bp, Homo sapiens
ggatctttatctggcttactt

SEQ ID NO: 107 - DNA, 21 bp, Homo sapiens
gatctttatctggcttacttt

SEQ ID NO: 108 - DNA, 21 bp, Homo sapiens
cggacaatcagatagttcaaa

SEQ ID NO: 109 - DNA, 21 bp, Homo sapiens
caaggagatgcggacatcaaa

SEQ ID NO: 110 - DNA, 21 bp, Homo sapiens
tgcccaacgggcacttaagtt

SEQ ID NO: 111 - DNA, 21 bp, Homo sapiens
agcagtcgagcccgtatttca

SEQ ID NO: 112 - DNA, 21 bp, Homo sapiens
gactcctccatcaccttcatt

SEQ ID NO: 113 - DNA, 21 bp, Homo sapiens
gtcctggttcctctgataaga

SEQ ID NO: 114 - DNA, 21 bp, Homo sapiens
tcctggttcctctgataagat

SEQ ID NO: 115 - DNA, 21 bp, Homo sapiens
ccagcagccaagcagaaagaa

SEQ ID NO: 116 - DNA, 21 bp, Homo sapiens
tagaggacagaaagcaagaaa

SEQ ID NO: 117 - DNA, 21 bp, Homo sapiens
caaagcaaccagcacaattct

SEQ ID NO: 118 - DNA, 21 bp, Homo sapiens
tggagggccagcttgtaaaga

SEQ ID NO: 119 - DNA, 21 bp, Homo sapiens
ggagggccagcttgtaaagat

SEQ ID NO: 120 - DNA, 21 bp, Homo sapiens
tttagctgtaatgtctttact

SEQ ID NO: 121 - DNA, 21 bp, Homo sapiens
gggaactcctcctccatttga

SEQ ID NO: 122 - DNA, 21 bp, Homo sapiens
tttgagcaacttgggaacaat

SEQ ID NO: 123 - DNA, 21 bp, Homo sapiens
ctctctttctctctctttctt

SEQ ID NO: 124 - DNA, 21 bp, Homo sapiens
tctctttctctctctttctta

SEQ ID NO: 125 - DNA, 21 bp, Homo sapiens
ttctctctctttcttaattgt

SEQ ID NO: 126 - DNA, 21 bp, Homo sapiens
tctctctctttcttaattgta

SEQ ID NO: 127 - DNA, 21 bp, Homo sapiens
ctctctctttcttaattgtat

SEQ ID NO: 128 - DNA, 21 bp, Homo sapiens
gctatgggctgtatttattgt

SEQ ID NO: 129 - DNA, 21 bp, Homo sapiens
gctgtatttattgtgaatcta

SEQ ID NO: 130 - DNA, 21 bp, Homo sapiens
gcgcggtcaaagtgcaatgat

SEQ ID NO: 131 - DNA, 21 bp, Homo sapiens
cgcggtcaaagtgcaatgatt

SEQ ID NO: 132 - DNA, 21 bp, Homo sapiens
ttcagttcggttggctaaaca

SEQ ID NO: 133 - DNA, 21 bp, Homo sapiens
tcctatctttgtatgttaaga

SEQ ID NO: 134 - DNA, 21 bp, Homo sapiens
atcaaccaacacaagatcaaa

SEQ ID NO: 135 - DNA, 21 bp, Homo sapiens
tttaaagaacccacaaactat

SEQ ID NO: 136 - DNA, 21 bp, Homo sapiens
gaacccacaaactattataca

SEQ ID NO: 137 - DNA, 21 bp, Homo sapiens
acccacaaactattatacata

SEQ ID NO: 138 - DNA, 21 bp, Homo sapiens
ttaaatatcctgcacttgtta

SEQ ID NO: 139 - DNA, 21 bp, Homo sapiens
gaatacgcatatcccttcttt

SEQ ID NO: 140 - DNA, 21 bp, Homo sapiens
tacgcatatcccttctttgtt

SEQ ID NO: 141 - DNA, 21 bp, Homo sapiens
aggtcaatgccaacgaacaaa

SEQ ID NO: 142 - DNA, 21 bp, Homo sapiens
tgaccctcaccagcattatca

SEQ ID NO: 143 - DNA, 21 bp, Homo sapiens
cgctcagcaagttggtagttt

SEQ ID NO: 144 - DNA, 21 bp, Homo sapiens
ccctgtcgactgccttataca

SEQ ID NO: 145 - DNA, 21 bp, Homo sapiens
cccgtcccgtcaaacaagttt

SEQ ID NO: 146 - DNA, 21 bp, Homo sapiens
gtcccgtcaaacaagtttctt

SEQ ID NO: 147 - DNA, 21 bp, Homo sapiens
cgtcaaacaagtttcttctta

SEQ ID NO: 148 - DNA, 21 bp, Homo sapiens
acgagtatctctatatatagt

SEQ ID NO: 149 - DNA, 21 bp, Homo sapiens
cgagtatctctatatatagta

SEQ ID NO: 150 - DNA, 21 bp, Homo sapiens
cgtctctgcactgagatacat

SEQ ID NO: 151 - DNA, 21 bp, Homo sapiens
gtctctgcactgagatacata

SEQ ID NO: 152 - DNA, 21 bp, Homo sapiens
tctgcactgagatacataaga

SEQ ID NO: 153 - DNA, 21 bp, Homo sapiens
ctgcactgagatacataagaa

SEQ ID NO: 154 - DNA, 21 bp, Homo sapiens
tgcactgagatacataagaaa

SEQ ID NO: 155 - DNA, 21 bp, Homo sapiens
actgagatacataagaaacaa

SEQ ID NO: 156 - DNA, 21 bp, Homo sapiens
cataagaaacaagggtagttt

SEQ ID NO: 157 - DNA, 21 bp, Homo sapiens
cgcttctgttataccatcttt

SEQ ID NO: 158 - DNA, 21 bp, Homo sapiens
ctctccggcttctccattgaa

SEQ ID NO: 159 - DNA, 21 bp, Homo sapiens
tctccggcttctccattgaat

SEQ ID NO: 160 - DNA, 21 bp, Homo sapiens
tctccattgaatggctaatgt

SEQ ID NO: 161 - DNA, 21 bp, Homo sapiens
ctggatctttatctggcttac

SEQ ID NO: 162 - DNA, 21 bp, Homo sapiens
ctttgtccacactccggaatc

SEQ ID NO: 163 - DNA, 21 bp, Homo sapiens
tctctaaagaagtcaggaaag

SEQ ID NO: 164 - DNA, 21 bp, Homo sapiens
gaagaagaggttcctcgaaag

SEQ ID NO: 165 - DNA, 21 bp, Homo sapiens
acttgggaacaatttggtaac

SEQ ID NO: 166 - DNA, 21 bp, Homo sapiens
gcttgtctttctgtgccttac

SEQ ID NO: 167 - DNA, 21 bp, Homo sapiens
ttcaacatctctcatcctatc

SEQ ID NO: 168 - DNA, 21 bp, Homo sapiens
caagtttcttcttaggctaag

SEQ ID NO: 169 - DNA, 21 bp, Homo sapiens
tttcagtgtcctagcggaatg

SEQ ID NO: 170 - DNA, 21 bp, Homo sapiens
catcacattggagtcacaatc

SEQ ID NO: 171 - DNA, 21 bp, Homo sapiens
ggacaatcagatagttcaaac

SEQ ID NO: 172 - DNA, 21 bp, Homo sapiens
ctgttgatgacgtgttctatc

SEQ ID NO: 173 - DNA, 21 bp, Homo sapiens
aggactcactgaaggagtttg

SEQ ID NO: 174 - DNA, 21 bp, Homo sapiens
tgaaggagtttgtgcagtttg

SEQ ID NO: 175 - DNA, 21 bp, Homo sapiens
ttcctatgcctgattccaaat

SEQ ID NO: 176 - DNA, 21 bp, Homo sapiens
tcctatgcctgattccaaatc

SEQ ID NO: 177 - DNA, 21 bp, Homo sapiens
actcctccatcaccttcattc

SEQ ID NO: 178 - DNA, 21 bp, Homo sapiens
agtcctggttcctctgataag

SEQ ID NO: 179 - DNA, 21 bp, Homo sapiens
agcagccaagcagaaagaaac

SEQ ID NO: 180 - DNA, 21 bp, Homo sapiens
tgtcaagtagaggacagaaag

SEQ ID NO: 181 - DNA, 21 bp, Homo sapiens
agaggacagaaagcaagaaag

SEQ ID NO: 182 - DNA, 21 bp, Homo sapiens
acaaagcaaccagcacaattc

SEQ ID NO: 183 - DNA, 21 bp, Homo sapiens
atgggaactcctcctccattt

SEQ ID NO: 184 - DNA, 21 bp, Homo sapiens
tgggaactcctcctccatttg

SEQ ID NO: 185 - DNA, 21 bp, Homo sapiens
gtccttgtgcctctctctttc

SEQ ID NO: 186 - DNA, 21 bp, Homo sapiens
ctctctctttctctctctttc

SEQ ID NO: 187 - DNA, 21 bp, Homo sapiens
tctctctttcttaattgtatg

SEQ ID NO: 188 - DNA, 21 bp, Homo sapiens
gggctgtatttattgtgaatc

SEQ ID NO: 189 - DNA, 21 bp, Homo sapiens
tttcagttcggttggctaaac

SEQ ID NO: 190 - DNA, 21 bp, Homo sapiens
ttcctatctttgtatgttaag

SEQ ID NO: 191 - DNA, 21 bp, Homo sapiens
tttcgtcgttcgttctgtttc

SEQ ID NO: 192 - DNA, 21 bp, Homo sapiens
ccacaaactattatacataag

SEQ ID NO: 193 - DNA, 21 bp, Homo sapiens
taaatatcctgcacttgttag

SEQ ID NO: 194 - DNA, 21 bp, Homo sapiens
atcctgcacttgttaggaata

SEQ ID NO: 195 - DNA, 21 bp, Homo sapiens
tcctgcacttgttaggaatac

SEQ ID NO: 196 - DNA, 21 bp, Homo sapiens
acttgttaggaatacgcatat

SEQ ID NO: 197 - DNA, 21 bp, Homo sapiens
cttgttaggaatacgcatatc

SEQ ID NO: 198 - DNA, 21 bp, Homo sapiens
ggtcaatgccaacgaacaaac

SEQ ID NO: 199 - DNA, 21 bp, Homo sapiens
acttgaccctcaccagcatta

SEQ ID NO: 200 - DNA, 21 bp, Homo sapiens
gctcagcaagttggtagtttc

SEQ ID NO: 201 - DNA, 21 bp, Homo sapiens
ccgtcccgtcaaacaagtttc

SEQ ID NO: 202 - DNA, 21 bp, Homo sapiens
gtcaaacaagtttcttcttag

SEQ ID NO: 203 - DNA, 21 bp, Homo sapiens
ctctgcactgagatacataag

SEQ ID NO: 204 - DNA, 21 bp, Homo sapiens
gcactgagatacataagaaac

SEQ ID NO: 205 - DNA, 21 bp, Homo sapiens
taagaaacaagggtagtttac

SEQ ID NO: 206 - DNA, 21 bp, Homo sapiens
gcttctgttataccatctttg

SEQ ID NO: 207 - DNA, 21 bp, Homo sapiens
ctccggcttctccattgaatg

SEQ ID NO: 208 - DNA, 21 bp, Homo sapiens
cttctccattgaatggctaat

SEQ ID NO: 209 - DNA, 21 bp, Homo sapiens
ttctccattgaatggctaatg

SEQ ID NO: 210 - DNA, 21 bp, Homo sapiens
attgaatggctaatgtgtatg

SEQ ID NO: 211 - DNA, 21 bp, Homo sapiens
cagtcctggttcctctgataa

SEQ ID NO: 212 - DNA, 21 bp, Homo sapiens
ctgtaatgtctttactcttta

SEQ ID NO: 213 - DNA, 21 bp, Homo sapiens
ttgagcaacttgggaacaatt

SEQ ID NO: 214 - DNA, 21 bp, Homo sapiens
tgagcaacttgggaacaattt

SEQ ID NO: 215 - DNA, 21 bp, Homo sapiens
gagcaacttgggaacaatttg

SEQ ID NO: 216 - DNA, 21 bp, Homo sapiens
ctctttctctctctttcttaa

SEQ ID NO: 217 - DNA, 21 bp, Homo sapiens
tctttctctctctttcttaat

SEQ ID NO: 218 - DNA, 21 bp, Homo sapiens
tttctctctctttcttaattg

SEQ ID NO: 219 - DNA, 21 bp, Homo sapiens
ggtgggctatgggctgtattt

SEQ ID NO: 220 - DNA, 21 bp, Homo sapiens
ggctatgggctgtatttattg

SEQ ID NO: 221 - DNA, 21 bp, Homo sapiens
gcggtcaaagtgcaatgattt

SEQ ID NO: 222 - DNA, 21 bp, Homo sapiens
ctgaaggaaacgttcacattt

SEQ ID NO: 223 - DNA, 21 bp, Homo sapiens
accctggtgcttcttacatta

SEQ ID NO: 224 - DNA, 21 bp, Homo sapiens
ccctggtgcttcttacattat

SEQ ID NO: 225 - DNA, 21 bp, Homo sapiens
ggtgcttcttacattataaag

SEQ ID NO: 226 - DNA, 21 bp, Homo sapiens
ttaaagaacccacaaactatt

SEQ ID NO: 227 - DNA, 21 bp, Homo sapiens
taaagaacccacaaactatta

SEQ ID NO: 228 - DNA, 21 bp, Homo sapiens
agaacccacaaactattatac

SEQ ID NO: 229 - DNA, 21 bp, Homo sapiens
cccacaaactattatacataa

SEQ ID NO: 230 - DNA, 21 bp, Homo sapiens
atcccttctttgttgagttta

SEQ ID NO: 231 - DNA, 21 bp, Homo sapiens
tcccttctttgttgagtttaa

SEQ ID NO: 232 - DNA, 21 bp, Homo sapiens
cccttctttgttgagtttaac

SEQ ID NO: 233 - DNA, 21 bp, Homo sapiens
cttgaccctcaccagcattat

SEQ ID NO: 234 - DNA, 21 bp, Homo sapiens
ttgaccctcaccagcattatc

SEQ ID NO: 235 - DNA, 21 bp, Homo sapiens
agccctgtcgactgccttata

SEQ ID NO: 236 - DNA, 21 bp, Homo sapiens
aggctaagaaacgcagtatat

SEQ ID NO: 237 - DNA, 21 bp, Homo sapiens
gctaagaaacgcagtatatac

SEQ ID NO: 238 - DNA, 21 bp, Homo sapiens
gtatatacgagtatctctata

SEQ ID NO: 239 - DNA, 21 bp, Homo sapiens
tacgagtatctctatatatag

SEQ ID NO: 240 - DNA, 21 bp, Homo sapiens
tctctgcactgagatacataa

SEQ ID NO: 241 - DNA, 21 bp, Homo sapiens
ataagaaacaagggtagttta

SEQ ID NO: 242 - DNA, 21 bp, Homo sapiens
ccgtcactgcgcttctgttat

SEQ ID NO: 243 - DNA, 21 bp, Homo sapiens
cgtcactgcgcttctgttata

SEQ ID NO: 244 - DNA, 21 bp, Homo sapiens
gtcactgcgcttctgttatac

SEQ ID NO: 245 - DNA, 21 bp, Homo sapiens
tggctaatgtgtatgtgaaat

SEQ ID NO: 246 - DNA, 21 bp, Homo sapiens
ggctaatgtgtatgtgaaata

SEQ ID NO: 247 - DNA, 21 bp, Homo sapiens
ctttctctctctttcttaatt

SEQ ID NO: 248 - DNA, 21 bp, Homo sapiens
gtgggctatgggctgtattta

SEQ ID NO: 249 - DNA, 21 bp, Homo sapiens
tgggctatgggctgtatttat

SEQ ID NO: 250 - DNA, 21 bp, Homo sapiens
gggctatgggctgtatttatt

SEQ ID NO: 251 - DNA, 21 bp, Homo sapiens
cctggtgcttcttacattata

SEQ ID NO: 252 - DNA, 21 bp, Homo sapiens
ctggtgcttcttacattataa

SEQ ID NO: 253 - DNA, 21 bp, Homo sapiens
tggtgcttcttacattataaa

SEQ ID NO: 254 - DNA, 21 bp, Homo sapiens
ggctaagaaacgcagtatata

SEQ ID NO: 255 - DNA, 21 bp, Homo sapiens
gctaatgtgtatgtgaaataa

SEQ ID NO: 256 - DNA, 57 bp, Artificial Sequence
  Description of Artificial Sequence Synthetic oligonucleotide
ccggcctgttgatgacgtgttctatctcgagatagaacacgtcatcaacaggttttt

SEQ ID NO: 257 - DNA, 57 bp, Artificial Sequence
  Description of Artificial Sequence Synthetic oligonucleotide
ccggcacatcacattggagtcacaactcgagttgtgactccaatgtgatgtgttttt

SEQ ID NO: 258 - DNA, 58 bp, Artificial Sequence
  Description of Artificial Sequence Synthetic oligonucleotide
ccggccggacaatcagatagttcaactcgagttgaactatctgattgtccggtttttg

SEQ ID NO: 259 - DNA, 57 bp, Artificial Sequence
  Description of Artificial Sequence Synthetic oligonucleotide
ccgggcagtctcagtcctggttcctctcgagaggaaccaggactgagactgcttttt

SEQ ID NO: 260 - DNA, 58 bp, Artificial Sequence
  Description of Artificial Sequence Synthetic oligonucleotide
ccggcccaacgggcacttaagtttcctcgaggaaacttaagtgcccgttgggtttttg

SEQ ID NO: 261 - DNA, 57 bp, Artificial Sequence
  Description of Artificial Sequence Synthetic oligonucleotide
ccggactggatctttatctggcttactcgagtaagccagataaagatccagtttttt

SEQ ID NO: 262 - DNA, 58 bp, Artificial Sequence
  Description of Artificial Sequence Synthetic oligonucleotide
ccggatctttatctggcttactttgctcgagcaaagtaagccagataaagattttttg

SEQ ID NO: 263 - DNA, 58 bp, Artificial Sequence
  Description of Artificial Sequence Synthetic oligonucleotide
ccggtaggctaagaaacgcagtatactcgagtatactgcgtttcttagcctatttttg

SEQ ID NO: 264 - DNA, 58 bp, Artificial Sequence
  Description of Artificial Sequence Synthetic oligonucleotide
ccggtggacctggtcatggtgatttctcgagaaatcaccatgaccaggtccatttttg

SEQ ID NO: 265 - DNA, 58 bp, Artificial Sequence
  Description of Artificial Sequence Synthetic oligonucleotide
ccggggatgagttccacccgttcatctcgagatgaacgggtggaactcatcctttttg

SEQ ID NO: 266 - DNA, 58 bp, Artificial Sequence
  Description of Artificial Sequence Synthetic oligonucleotide
ccggtttctcctacacctggttcaactcgagttgaaccaggtgtaggagaaatttttg

SEQ ID NO: 267 - DNA, 58 bp, Artificial Sequence
  Description of Artificial Sequence Synthetic oligonucleotide
ccggttctcctacacctggttcaacctcgaggttgaaccaggtgtaggagaatttttg

SEQ ID NO: 268 - DNA, 58 bp, Artificial Sequence
  Description of Artificial Sequence Synthetic oligonucleotide
ccgggcggaagcgcaagtacttcaactcgagttgaagtacttgcgcttccgctttttg

SEQ ID NO: 269 - DNA, 58 bp, Artificial Sequence
  Description of Artificial Sequence Synthetic oligonucleotide
ccggcggaagcgcaagtacttcaagctcgagcttgaagtacttgcgcttccgtttttg

SEQ ID NO: 270 - DNA, 58 bp, Artificial Sequence
  Description of Artificial Sequence Synthetic oligonucleotide
ccggggaagcgcaagtacttcaagactcgagtcttgaagtacttgcgcttcctttttg

SEQ ID NO: 271 - DNA, 58 bp, Artificial Sequence
  Description of Artificial Sequence Synthetic oligonucleotide
ccgggaagcgcaagtacttcaagaactcgagttcttgaagtacttgcgcttctttttg

SEQ ID NO: 272 - DNA, 58 bp, Artificial Sequence
  Description of Artificial Sequence Synthetic oligonucleotide
ccggagcccgagatcaagcagaagtctcgagacttctgcttgatctcgggcttttttg

SEQ ID NO: 273 - DNA, 58 bp, Artificial Sequence
  Description of Artificial Sequence Synthetic oligonucleotide
ccggctggacctggtcatggtgattctcgagaatcaccatgaccaggtccagtttttg

SEQ ID NO: 274 - DNA, 58 bp, Artificial Sequence
  Description of Artificial Sequence Synthetic oligonucleotide
ccggtgcgtccagccacatcacattctcgagaatgtgatgtggctggacgcatttttg

SEQ ID NO: 275 - DNA, 58 bp, Artificial Sequence
  Description of Artificial Sequence Synthetic oligonucleotide
ccggacatcacattggagtcacaatctcgagattgtgactccaatgtgatgttttttg

SEQ ID NO: 276 - DNA, 58 bp, Artificial Sequence
  Description of Artificial Sequence Synthetic oligonucleotide
ccggatcacattggagtcacaatcactcgagtgattgtgactccaatgtgattttttg

SEQ ID NO: 277 - DNA, 58 bp, Artificial Sequence
  Description of Artificial Sequence Synthetic oligonucleotide
ccggcattggagtcacaatcaaagactcgagtctttgattgtgactccaatgtttttg

SEQ ID NO: 278 - DNA, 58 bp, Artificial Sequence
  Description of Artificial Sequence Synthetic oligonucleotide
ccggttggagtcacaatcaaagaacctcgaggttctttgattgtgactccaatttttg

SEQ ID NO: 279 - DNA, 58 bp, Artificial Sequence
  Description of Artificial Sequence Synthetic oligonucleotide
ccggtggagtcacaatcaaagaactctcgagagttctttgattgtgactccatttttg

SEQ ID NO: 280 - DNA, 58 bp, Artificial Sequence
  Description of Artificial Sequence Synthetic oligonucleotide
ccggacaatcaaagaactggatcttctcgagaagatccagttctttgattgttttttg

SEQ ID NO: 281 - DNA, 58 bp, Artificial Sequence
  Description of Artificial Sequence Synthetic oligonucleotide
ccggtggatctttatctggcttactctcgagagtaagccagataaagatccatttttg

SEQ ID NO: 282 - DNA, 58 bp, Artificial Sequence
  Description of Artificial Sequence Synthetic oligonucleotide
ccggtctttatctggcttactttgtctcgagacaaagtaagccagataaagatttttg

SEQ ID NO: 283 - DNA, 58 bp, Artificial Sequence
  Description of Artificial Sequence Synthetic oligonucleotide
ccggctttatctggcttactttgtcctcgaggacaaagtaagccagataaagtttttg

SEQ ID NO: 284 - DNA, 58 bp, Artificial Sequence
  Description of Artificial Sequence Synthetic oligonucleotide
ccggacactccggaatccggacaatctcgagattgtccggattccggagtgttttttg

SEQ ID NO: 285 - DNA, 58 bp, Artificial Sequence
  Description of Artificial Sequence Synthetic oligonucleotide
ccggactccggaatccggacaatcactcgagtgattgtccggattccggagttttttg

SEQ ID NO: 286 - DNA, 58 bp, Artificial Sequence
  Description of Artificial Sequence Synthetic oligonucleotide
ccggccggaatccggacaatcagatctcgagatctgattgtccggattccggtttttg

SEQ ID NO: 287 - DNA, 58 bp, Artificial Sequence
  Description of Artificial Sequence Synthetic oligonucleotide
ccggcggaatccggacaatcagatactcgagtatctgattgtccggattccgtttttg

SEQ ID NO: 288 - DNA, 58 bp, Artificial Sequence
  Description of Artificial Sequence Synthetic oligonucleotide
ccgggaatccggacaatcagatagtctcgagactatctgattgtccggattctttttg

SEQ ID NO: 289 - DNA, 58 bp, Artificial Sequence
  Description of Artificial Sequence Synthetic oligonucleotide
ccggatccggacaatcagatagttcctcgaggaactatctgattgtccggattttttg

SEQ ID NO: 290 - DNA, 58 bp, Artificial Sequence
  Description of Artificial Sequence Synthetic oligonucleotide
ccggtccggacaatcagatagttcactcgagtgaactatctgattgtccggatttttg

SEQ ID NO: 291 - DNA, 58 bp, Artificial Sequence
  Description of Artificial Sequence Synthetic oligonucleotide
ccggagcaaggagatgcggacatcactcgagtgatgtccgcatctccttgcttttttg

SEQ ID NO: 292 - DNA, 58 bp, Artificial Sequence
  Description of Artificial Sequence Synthetic oligonucleotide
ccgggcaaggagatgcggacatcaactcgagttgatgtccgcatctccttgctttttg

SEQ ID NO: 293 - DNA, 58 bp, Artificial Sequence
  Description of Artificial Sequence Synthetic oligonucleotide
ccggacttaagtttccaggactgttctcgagaacagtcctggaaacttaagttttttg

SEQ ID NO: 294 - DNA, 58 bp, Artificial Sequence
  Description of Artificial Sequence Synthetic oligonucleotide
ccggcttaagtttccaggactgtttctcgagaaacagtcctggaaacttaagtttttg

SEQ ID NO: 295 - DNA, 58 bp, Artificial Sequence
  Description of Artificial Sequence Synthetic oligonucleotide
ccggtgacggagctggtgagagtatctcgagatactctcaccagctccgtcatttttg

SEQ ID NO: 296 - DNA, 58 bp, Artificial Sequence
  Description of Artificial Sequence Synthetic oligonucleotide
ccggacggagctggtgagagtatcactcgagtgatactctcaccagctccgttttttg

SEQ ID NO: 297 - DNA, 58 bp, Artificial Sequence
  Description of Artificial Sequence Synthetic oligonucleotide
ccggtggagagtcccagctactacactcgagtgtagtagctgggactctccatttttg

SEQ ID NO: 298 - DNA, 58 bp, Artificial Sequence
  Description of Artificial Sequence Synthetic oligonucleotide
ccggggagagtcccagctactacaactcgagttgtagtagctgggactctcctttttg

SEQ ID NO: 299 - DNA, 58 bp, Artificial Sequence
  Description of Artificial Sequence Synthetic oligonucleotide
ccgggagagtcccagctactacaacctcgaggttgtagtagctgggactctctttttg

SEQ ID NO: 300 - DNA, 58 bp, Artificial Sequence
  Description of Artificial Sequence Synthetic oligonucleotide
ccggagagtcccagctactacaacactcgagtgttgtagtagctgggactcttttttg

SEQ ID NO: 301 - DNA, 58 bp, Artificial Sequence
  Description of Artificial Sequence Synthetic oligonucleotide
ccgggagtcccagctactacaacatctcgagatgttgtagtagctgggactctttttg

SEQ ID NO: 302 - DNA, 58 bp, Artificial Sequence
  Description of Artificial Sequence Synthetic oligonucleotide
ccggagtcccagctactacaacatcctcgaggatgttgtagtagctgggacttttttg

SEQ ID NO: 303 - DNA, 58 bp, Artificial Sequence
  Description of Artificial Sequence Synthetic oligonucleotide
ccgggtcccagctactacaacatcactcgagtgatgttgtagtagctgggactttttg

SEQ ID NO: 304 - DNA, 58 bp, Artificial Sequence
  Description of Artificial Sequence Synthetic oligonucleotide
ccggtcccagctactacaacatcaactcgagttgatgttgtagtagctgggatttttg

SEQ ID NO: 305 - DNA, 58 bp, Artificial Sequence
  Description of Artificial Sequence Synthetic oligonucleotide
ccggcccagctactacaacatcaacctcgaggttgatgttgtagtagctgggtttttg

SEQ ID NO: 306 - DNA, 58 bp, Artificial Sequence
  Description of Artificial Sequence Synthetic oligonucleotide
ccgggtccatcgatgacagtgagatctcgagatctcactgtcatcgatggactttttg

SEQ ID NO: 307 - DNA, 58 bp, Artificial Sequence
  Description of Artificial Sequence Synthetic oligonucleotide
ccggtgagatggagagccctgttgactcgagtcaacagggctctccatctcatttttg

SEQ ID NO: 308 - DNA, 58 bp, Artificial Sequence
  Description of Artificial Sequence Synthetic oligonucleotide
ccgggagatggagagccctgttgatctcgagatcaacagggctctccatctctttttg

SEQ ID NO: 309 - DNA, 58 bp, Artificial Sequence
  Description of Artificial Sequence Synthetic oligonucleotide
ccggagatggagagccctgttgatgctcgagcatcaacagggctctccatcttttttg

SEQ ID NO: 310 - DNA, 58 bp, Artificial Sequence
  Description of Artificial Sequence Synthetic oligonucleotide
ccgggatggagagccctgttgatgactcgagtcatcaacagggctctccatctttttg

SEQ ID NO: 311 - DNA, 58 bp, Artificial Sequence
  Description of Artificial Sequence Synthetic oligonucleotide
ccgggccctgttgatgacgtgttctctcgagagaacacgtcatcaacagggctttttg

SEQ ID NO: 312 - DNA, 58 bp, Artificial Sequence
  Description of Artificial Sequence Synthetic oligonucleotide
ccggccctgttgatgacgtgttctactcgagtagaacacgtcatcaacagggtttttg

SEQ ID NO: 313 - DNA, 58 bp, Artificial Sequence
  Description of Artificial Sequence Synthetic oligonucleotide
ccggttctctaaagaagtcaggaaactcgagtttcctgacttctttagagaatttttg

SEQ ID NO: 314 - DNA, 58 bp, Artificial Sequence
  Description of Artificial Sequence Synthetic oligonucleotide
ccggagtcaggaaagctggacttctctcgagagaagtccagctttcctgacttttttg

SEQ ID NO: 315 - DNA, 58 bp, Artificial Sequence
  Description of Artificial Sequence Synthetic oligonucleotide
ccggcaggactcactgaaggagtttctcgagaaactccttcagtgagtcctgtttttg

SEQ ID NO: 316 - DNA, 58 bp, Artificial Sequence
  Description of Artificial Sequence Synthetic oligonucleotide
ccggggactcactgaaggagtttgtctcgagacaaactccttcagtgagtcctttttg

SEQ ID NO: 317 - DNA, 58 bp, Artificial Sequence
  Description of Artificial Sequence Synthetic oligonucleotide
ccgggactcactgaaggagtttgtgctcgagcacaaactccttcagtgagtctttttg

SEQ ID NO: 318 - DNA, 58 bp, Artificial Sequence
  Description of Artificial Sequence Synthetic oligonucleotide
ccggctgaaggagtttgtgcagtttctcgagaaactgcacaaactccttcagtttttg

SEQ ID NO: 319 - DNA, 58 bp, Artificial Sequence
  Description of Artificial Sequence Synthetic oligonucleotide
ccgggaaggagtttgtgcagtttgtctcgagacaaactgcacaaactccttctttttg

SEQ ID NO: 320 - DNA, 58 bp, Artificial Sequence
  Description of Artificial Sequence Synthetic oligonucleotide
ccggcttcctatgcctgattccaaactcgagtttggaatcaggcataggaagtttttg

SEQ ID NO: 321 - DNA, 58 bp, Artificial Sequence
  Description of Artificial Sequence Synthetic oligonucleotide
ccggcctatgcctgattccaaatccctcgagggatttggaatcaggcataggtttttg

SEQ ID NO: 322 - DNA, 58 bp, Artificial Sequence
  Description of Artificial Sequence Synthetic oligonucleotide
ccggtgactcctccatcaccttcatctcgagatgaaggtgatggaggagtcatttttg

SEQ ID NO: 323 - DNA, 58 bp, Artificial Sequence
  Description of Artificial Sequence Synthetic oligonucleotide
ccgggggacggcaactttctgaacactcgagtgttcagaaagttgccgtccctttttg

SEQ ID NO: 324 - DNA, 58 bp, Artificial Sequence
  Description of Artificial Sequence Synthetic oligonucleotide
ccggggacggcaactttctgaacatctcgagatgttcagaaagttgccgtcctttttg

SEQ ID NO: 325 - DNA, 58 bp, Artificial Sequence
  Description of Artificial Sequence Synthetic oligonucleotide
ccgggacggcaactttctgaacatcctcgaggatgttcagaaagttgccgtctttttg

SEQ ID NO: 326 - DNA, 58 bp, Artificial Sequence
  Description of Artificial Sequence Synthetic oligonucleotide
ccggctcagtcctggttcctctgatctcgagatcagaggaaccaggactgagtttttg

SEQ ID NO: 327 - DNA, 58 bp, Artificial Sequence
  Description of Artificial Sequence Synthetic oligonucleotide
ccggtcagtcctggttcctctgatactcgagtatcagaggaaccaggactgatttttg

SEQ ID NO: 328 - DNA, 58 bp, Artificial Sequence
  Description of Artificial Sequence Synthetic oligonucleotide
ccggcctggttcctctgataagatcctcgaggatcttatcagaggaaccaggtttttg

SEQ ID NO: 329 - DNA, 58 bp, Artificial Sequence
  Description of Artificial Sequence Synthetic oligonucleotide
ccggtcctctgataagatcgacaaactcgagtttgtcgatcttatcagaggatttttg

SEQ ID NO: 330 - DNA, 58 bp, Artificial Sequence
  Description of Artificial Sequence Synthetic oligonucleotide
ccggagaagaagaggttcctcgaaactcgagtttcgaggaacctcttcttcttttttg

SEQ ID NO: 331 - DNA, 58 bp, Artificial Sequence
  Description of Artificial Sequence Synthetic oligonucleotide
ccggaccagcagccaagcagaaagactcgagtctttctgcttggctgctggttttttg

SEQ ID NO: 332 - DNA, 58 bp, Artificial Sequence
  Description of Artificial Sequence Synthetic oligonucleotide
ccgggcagccaagcagaaagaaacactcgagtgtttctttctgcttggctgctttttg

SEQ ID NO: 333 - DNA, 58 bp, Artificial Sequence
  Description of Artificial Sequence Synthetic oligonucleotide
ccggcagccaagcagaaagaaacacctcgaggtgtttctttctgcttggctgtttttg

SEQ ID NO: 334 - DNA, 58 bp, Artificial Sequence
  Description of Artificial Sequence Synthetic oligonucleotide
ccggacgcgacatggactctgtcaactcgagttgacagagtccatgtcgcgttttttg

SEQ ID NO: 335 - DNA, 58 bp, Artificial Sequence
  Description of Artificial Sequence Synthetic oligonucleotide
ccgggcgacatggactctgtcaagtctcgagacttgacagagtccatgtcgctttttg

SEQ ID NO: 336 - DNA, 58 bp, Artificial Sequence
  Description of Artificial Sequence Synthetic oligonucleotide
ccggcgacatggactctgtcaagtactcgagtacttgacagagtccatgtcgtttttg

SEQ ID NO: 337 - DNA, 58 bp, Artificial Sequence
  Description of Artificial Sequence Synthetic oligonucleotide
ccgggacatggactctgtcaagtagctcgagctacttgacagagtccatgtctttttg

SEQ ID NO: 338 - DNA, 58 bp, Artificial Sequence
  Description of Artificial Sequence Synthetic oligonucleotide
ccggacatggactctgtcaagtagactcgagtctacttgacagagtccatgttttttg

SEQ ID NO: 339 - DNA, 58 bp, Artificial Sequence
  Description of Artificial Sequence Synthetic oligonucleotide
ccgggtagaggacagaaagcaagaactcgagttcttgctttctgtcctctactttttg

SEQ ID NO: 340 - DNA, 58 bp, Artificial Sequence
  Description of Artificial Sequence Synthetic oligonucleotide
ccggagcaagaaaggatgcagaactctcgagagttctgcatcctttcttgcttttttg

SEQ ID NO: 341 - DNA, 58 bp, Artificial Sequence
  Description of Artificial Sequence Synthetic oligonucleotide
ccggagcaaccagcacaattctgaactcgagttcagaattgtgctggttgcttttttg

SEQ ID NO: 342 - DNA, 58 bp, Artificial Sequence
  Description of Artificial Sequence Synthetic oligonucleotide
ccgggagggccagcttgtaaagatgctcgagcatctttacaagctggccctctttttg

SEQ ID NO: 343 - DNA, 58 bp, Artificial Sequence
  Description of Artificial Sequence Synthetic oligonucleotide
ccggagggccagcttgtaaagatgactcgagtcatctttacaagctggcccttttttg

SEQ ID NO: 344 - DNA, 58 bp, Artificial Sequence
  Description of Artificial Sequence Synthetic oligonucleotide
ccgggggccagcttgtaaagatgatctcgagatcatctttacaagctggccctttttg

SEQ ID NO: 345 - DNA, 58 bp, Artificial Sequence
  Description of Artificial Sequence Synthetic oligonucleotide
ccggggccagcttgtaaagatgatgctcgagcatcatctttacaagctggcctttttg

SEQ ID NO: 346 - DNA, 58 bp, Artificial Sequence
  Description of Artificial Sequence Synthetic oligonucleotide
ccgggctagttgcgtgtcccagaatctcgagattctgggacacgcaactagctttttg

SEQ ID NO: 347 - DNA, 58 bp, Artificial Sequence
  Description of Artificial Sequence Synthetic oligonucleotide
ccggtcccagaatggagggtgttctctcgagagaacaccctccattctgggatttttg

SEQ ID NO: 348 - DNA, 58 bp, Artificial Sequence
  Description of Artificial Sequence Synthetic oligonucleotide
ccggctctgtacagtccgtaggaaactcgagtttcctacggactgtacagagtttttg

SEQ ID NO: 349 - DNA, 58 bp, Artificial Sequence
  Description of Artificial Sequence Synthetic oligonucleotide
ccggagtacttcaagaagcatgaaactcgagtttcatgcttcttgaagtacttttttg

SEQ ID NO: 350 - DNA, 58 bp, Artificial Sequence
  Description of Artificial Sequence Synthetic oligonucleotide
ccggtcacattggagtcacaatcaactcgagttgattgtgactccaatgtgatttttg

SEQ ID NO: 351 - DNA, 58 bp, Artificial Sequence
  Description of Artificial Sequence Synthetic oligonucleotide
ccggcacattggagtcacaatcaaactcgagtttgattgtgactccaatgtgtttttg

SEQ ID NO: 352 - DNA, 58 bp, Artificial Sequence
  Description of Artificial Sequence Synthetic oligonucleotide
ccggattggagtcacaatcaaagaactcgagttctttgattgtgactccaattttttg

SEQ ID NO: 353 - DNA, 58 bp, Artificial Sequence
  Description of Artificial Sequence Synthetic oligonucleotide
ccggggatctttatctggcttacttctcgagaagtaagccagataaagatcctttttg

SEQ ID NO: 354 - DNA, 58 bp, Artificial Sequence
  Description of Artificial Sequence Synthetic oligonucleotide
ccgggatctttatctggcttactttctcgagaaagtaagccagataaagatctttttg

SEQ ID NO: 355 - DNA, 58 bp, Artificial Sequence
  Description of Artificial Sequence Synthetic oligonucleotide
ccggcggacaatcagatagttcaaactcgagtttgaactatctgattgtccgtttttg

SEQ ID NO: 356 - DNA, 58 bp, Artificial Sequence
  Description of Artificial Sequence Synthetic oligonucleotide
ccggcaaggagatgcggacatcaaactcgagtttgatgtccgcatctccttgtttttg

SEQ ID NO: 357 - DNA, 58 bp, Artificial Sequence
  Description of Artificial Sequence Synthetic oligonucleotide
ccggtgcccaacgggcacttaagttctcgagaacttaagtgcccgttgggcatttttg

SEQ ID NO: 358 - DNA, 58 bp, Artificial Sequence
  Description of Artificial Sequence Synthetic oligonucleotide
ccgggcccaacgggcacttaagtttctcgagaaacttaagtgcccgttgggctttttg

SEQ ID NO: 359 - DNA, 58 bp, Artificial Sequence
  Description of Artificial Sequence Synthetic oligonucleotide
ccggagcagtcgagcccgtatttcactcgagtgaaatacgggctcgactgcttttttg

SEQ ID NO: 360 - DNA, 58 bp, Artificial Sequence
  Description of Artificial Sequence Synthetic oligonucleotide
ccgggactcctccatcaccttcattctcgagaatgaaggtgatggaggagtctttttg

SEQ ID NO: 361 - DNA, 58 bp, Artificial Sequence
  Description of Artificial Sequence Synthetic oligonucleotide
ccgggtcctggttcctctgataagactcgagtcttatcagaggaaccaggactttttg

SEQ ID NO: 362 - DNA, 58 bp, Artificial Sequence
  Description of Artificial Sequence Synthetic oligonucleotide
ccggtcctggttcctctgataagatctcgagatcttatcagaggaaccaggatttttg

SEQ ID NO: 363 - DNA, 58 bp, Artificial Sequence
  Description of Artificial Sequence Synthetic oligonucleotide
ccggccagcagccaagcagaaagaactcgagttctttctgcttggctgctggtttttg

SEQ ID NO: 364 - DNA, 58 bp, Artificial Sequence
  Description of Artificial Sequence Synthetic oligonucleotide
ccggcagcagccaagcagaaagaaactcgagtttctttctgcttggctgctgtttttg

SEQ ID NO: 365 - DNA, 58 bp, Artificial Sequence
  Description of Artificial Sequence Synthetic oligonucleotide
ccggtagaggacagaaagcaagaaactcgagtttcttgctttctgtcctctatttttg

SEQ ID NO: 366 - DNA, 58 bp, Artificial Sequence
  Description of Artificial Sequence Synthetic oligonucleotide
ccggcaaagcaaccagcacaattctctcgagagaattgtgctggttgctttgtttttg

SEQ ID NO: 367 - DNA, 58 bp, Artificial Sequence
  Description of Artificial Sequence Synthetic oligonucleotide
ccggtggagggccagcttgtaaagactcgagtctttacaagctggccctccatttttg

SEQ ID NO: 368 - DNA, 58 bp, Artificial Sequence
  Description of Artificial Sequence Synthetic oligonucleotide
ccggggagggccagcttgtaaagatctcgagatctttacaagctggccctcctttttg

SEQ ID NO: 369 - DNA, 58 bp, Artificial Sequence
  Description of Artificial Sequence Synthetic oligonucleotide
ccggtttagctgtaatgtctttactctcgagagtaaagacattacagctaaatttttg

SEQ ID NO: 370 - DNA, 58 bp, Artificial Sequence
  Description of Artificial Sequence Synthetic oligonucleotide
ccgggggaactcctcctccatttgactcgagtcaaatggaggaggagttccctttttg

SEQ ID NO: 371 - DNA, 58 bp, Artificial Sequence
  Description of Artificial Sequence Synthetic oligonucleotide
ccggtttgagcaacttgggaacaatctcgagattgttcccaagttgctcaaatttttg

SEQ ID NO: 372 - DNA, 58 bp, Artificial Sequence
  Description of Artificial Sequence Synthetic oligonucleotide
ccggctctctttctctctctttcttctcgagaagaaagagagagaaagagagtttttg

SEQ ID NO: 373 - DNA, 58 bp, Artificial Sequence
  Description of Artificial Sequence Synthetic oligonucleotide
ccggtctctttctctctctttcttactcgagtaagaaagagagagaaagagatttttg

SEQ ID NO: 374 - DNA, 58 bp, Artificial Sequence
  Description of Artificial Sequence Synthetic oligonucleotide
ccggttctctctctttcttaattgtctcgagacaattaagaaagagagagaatttttg

SEQ ID NO: 375 - DNA, 58 bp, Artificial Sequence
  Description of Artificial Sequence Synthetic oligonucleotide
ccggtctctctctttcttaattgtactcgagtacaattaagaaagagagagatttttg

SEQ ID NO: 376 - DNA, 58 bp, Artificial Sequence
  Description of Artificial Sequence Synthetic oligonucleotide
ccggctctctctttcttaattgtatctcgagatacaattaagaaagagagagtttttg

SEQ ID NO: 377 - DNA, 58 bp, Artificial Sequence
  Description of Artificial Sequence Synthetic oligonucleotide
ccgggctatgggctgtatttattgtctcgagacaataaatacagcccatagctttttg

SEQ ID NO: 378 - DNA, 58 bp, Artificial Sequence
  Description of Artificial Sequence Synthetic oligonucleotide
ccgggctgtatttattgtgaatctactcgagtagattcacaataaatacagctttttg

SEQ ID NO: 379 - DNA, 58 bp, Artificial Sequence
  Description of Artificial Sequence Synthetic oligonucleotide
ccgggcgcggtcaaagtgcaatgatctcgagatcattgcactttgaccgcgctttttg

SEQ ID NO: 380 - DNA, 58 bp, Artificial Sequence
  Description of Artificial Sequence Synthetic oligonucleotide
ccggcgcggtcaaagtgcaatgattctcgagaatcattgcactttgaccgcgtttttg

SEQ ID NO: 381 - DNA, 58 bp, Artificial Sequence
  Description of Artificial Sequence Synthetic oligonucleotide
ccggttcagttcggttggctaaacactcgagtgtttagccaaccgaactgaatttttg

SEQ ID NO: 382 - DNA, 58 bp, Artificial Sequence
  Description of Artificial Sequence Synthetic oligonucleotide
ccggtcctatctttgtatgttaagactcgagtcttaacatacaaagataggatttttg

SEQ ID NO: 383 - DNA, 58 bp, Artificial Sequence
  Description of Artificial Sequence Synthetic oligonucleotide
ccggatcaaccaacacaagatcaaactcgagtttgatcttgtgttggttgattttttg

SEQ ID NO: 384 - DNA, 58 bp, Artificial Sequence
  Description of Artificial Sequence Synthetic oligonucleotide
ccgggtgcttcttacattataaagtctcgagactttataatgtaagaagcactttttg

SEQ ID NO: 385 - DNA, 58 bp, Artificial Sequence
  Description of Artificial Sequence Synthetic oligonucleotide
ccggtttaaagaacccacaaactatctcgagatagtttgtgggttctttaaatttttg

SEQ ID NO: 386 - DNA, 58 bp, Artificial Sequence
  Description of Artificial Sequence Synthetic oligonucleotide
ccgggaacccacaaactattatacactcgagtgtataatagtttgtgggttctttttg

SEQ ID NO: 387 - DNA, 58 bp, Artificial Sequence
  Description of Artificial Sequence Synthetic oligonucleotide
ccggacccacaaactattatacatactcgagtatgtataatagtttgtgggttttttg

SEQ ID NO: 388 - DNA, 58 bp, Artificial Sequence
  Description of Artificial Sequence Synthetic oligonucleotide
ccggttaaatatcctgcacttgttactcgagtaacaagtgcaggatatttaatttttg

SEQ ID NO: 389 - DNA, 58 bp, Artificial Sequence
  Description of Artificial Sequence Synthetic oligonucleotide
ccgggaatacgcatatcccttctttctcgagaaagaagggatatgcgtattctttttg

SEQ ID NO: 390 - DNA, 58 bp, Artificial Sequence
  Description of Artificial Sequence Synthetic oligonucleotide
ccggtacgcatatcccttctttgttctcgagaacaaagaagggatatgcgtatttttg

SEQ ID NO: 391 - DNA, 58 bp, Artificial Sequence
  Description of Artificial Sequence Synthetic oligonucleotide
ccggaggtcaatgccaacgaacaaactcgagtttgttcgttggcattgaccttttttg

SEQ ID NO: 392 - DNA, 58 bp, Artificial Sequence
  Description of Artificial Sequence Synthetic oligonucleotide
ccggtgaccctcaccagcattatcactcgagtgataatgctggtgagggtcatttttg

SEQ ID NO: 393 - DNA, 58 bp, Artificial Sequence
  Description of Artificial Sequence Synthetic oligonucleotide
ccggcgctcagcaagttggtagtttctcgagaaactaccaacttgctgagcgtttttg

SEQ ID NO: 394 - DNA, 58 bp, Artificial Sequence
  Description of Artificial Sequence Synthetic oligonucleotide
ccggccctgtcgactgccttatacactcgagtgtataaggcagtcgacagggtttttg

SEQ ID NO: 395 - DNA, 58 bp, Artificial Sequence
  Description of Artificial Sequence Synthetic oligonucleotide
ccggcccgtcccgtcaaacaagtttctcgagaaacttgtttgacgggacgggtttttg

SEQ ID NO: 396 - DNA, 58 bp, Artificial Sequence
  Description of Artificial Sequence Synthetic oligonucleotide
ccgggtcccgtcaaacaagtttcttctcgagaagaaacttgtttgacgggactttttg

SEQ ID NO: 397 - DNA, 58 bp, Artificial Sequence
  Description of Artificial Sequence Synthetic oligonucleotide
ccggcgtcaaacaagtttcttcttactcgagtaagaagaaacttgtttgacgtttttg

SEQ ID NO: 398 - DNA, 58 bp, Artificial Sequence
  Description of Artificial Sequence Synthetic oligonucleotide
ccggacgagtatctctatatatagtctcgagactatatatagagatactcgttttttg

SEQ ID NO: 399 - DNA, 58 bp, Artificial Sequence
  Description of Artificial Sequence Synthetic oligonucleotide
ccggcgagtatctctatatatagtactcgagtactatatatagagatactcgtttttg

SEQ ID NO: 400 - DNA, 58 bp, Artificial Sequence
  Description of Artificial Sequence Synthetic oligonucleotide
ccggcgtctctgcactgagatacatctcgagatgtatctcagtgcagagacgtttttg

SEQ ID NO: 401 - DNA, 58 bp, Artificial Sequence
  Description of Artificial Sequence Synthetic oligonucleotide
ccgggtctctgcactgagatacatactcgagtatgtatctcagtgcagagactttttg

SEQ ID NO: 402 - DNA, 58 bp, Artificial Sequence
  Description of Artificial Sequence Synthetic oligonucleotide
ccggtctgcactgagatacataagactcgagtcttatgtatctcagtgcagatttttg

SEQ ID NO: 403 - DNA, 58 bp, Artificial Sequence
  Description of Artificial Sequence Synthetic oligonucleotide
ccggctgcactgagatacataagaactcgagttcttatgtatctcagtgcagtttttg

SEQ ID NO: 404 - DNA, 58 bp, Artificial Sequence
  Description of Artificial Sequence Synthetic oligonucleotide
ccggtgcactgagatacataagaaactcgagtttcttatgtatctcagtgcatttttg

SEQ ID NO: 405 - DNA, 58 bp, Artificial Sequence
  Description of Artificial Sequence Synthetic oligonucleotide
ccggactgagatacataagaaacaactcgagttgtttcttatgtatctcagttttttg

SEQ ID NO: 406 - DNA, 58 bp, Artificial Sequence
  Description of Artificial Sequence Synthetic oligonucleotide
ccggcataagaaacaagggtagtttctcgagaaactacccttgtttcttatgtttttg

SEQ ID NO: 407 - DNA, 58 bp, Artificial Sequence
  Description of Artificial Sequence Synthetic oligonucleotide
ccggcgcttctgttataccatctttctcgagaaagatggtataacagaagcgtttttg

SEQ ID NO: 408 - DNA, 58 bp, Artificial Sequence
  Description of Artificial Sequence Synthetic oligonucleotide
ccggctctccggcttctccattgaactcgagttcaatggagaagccggagagtttttg

SEQ ID NO: 409 - DNA, 58 bp, Artificial Sequence
  Description of Artificial Sequence Synthetic oligonucleotide
ccggtctccggcttctccattgaatctcgagattcaatggagaagccggagatttttg

SEQ ID NO: 410 - DNA, 58 bp, Artificial Sequence
  Description of Artificial Sequence Synthetic oligonucleotide
ccggtctccattgaatggctaatgtctcgagacattagccattcaatggagatttttg

SEQ ID NO: 411 - DNA, 58 bp, Artificial Sequence
  Description of Artificial Sequence Synthetic oligonucleotide
ccggctggatctttatctggcttacctcgaggtaagccagataaagatccagtttttg

SEQ ID NO: 412 - DNA, 58 bp, Artificial Sequence
  Description of Artificial Sequence Synthetic oligonucleotide
ccggctttgtccacactccggaatcctcgaggattccggagtgtggacaaagtttttg

SEQ ID NO: 413 - DNA, 58 bp, Artificial Sequence
  Description of Artificial Sequence Synthetic oligonucleotide
ccggtctctaaagaagtcaggaaagctcgagctttcctgacttctttagagatttttg

SEQ ID NO: 414 - DNA, 58 bp, Artificial Sequence
  Description of Artificial Sequence Synthetic oligonucleotide
ccgggaagaagaggttcctcgaaagctcgagctttcgaggaacctcttcttctttttg

SEQ ID NO: 415 - DNA, 58 bp, Artificial Sequence
  Description of Artificial Sequence Synthetic oligonucleotide
ccggacttgggaacaatttggtaacctcgaggttaccaaattgttcccaagttttttg

SEQ ID NO: 416 - DNA, 58 bp, Artificial Sequence
  Description of Artificial Sequence Synthetic oligonucleotide
ccgggcttgtctttctgtgccttacctcgaggtaaggcacagaaagacaagctttttg

SEQ ID NO: 417 - DNA, 58 bp, Artificial Sequence
  Description of Artificial Sequence Synthetic oligonucleotide
ccggttcaacatctctcatcctatcctcgaggataggatgagagatgttgaatttttg

SEQ ID NO: 418 - DNA, 58 bp, Artificial Sequence
  Description of Artificial Sequence Synthetic oligonucleotide
ccggcaagtttcttcttaggctaagctcgagcttagcctaagaagaaacttgtttttg

SEQ ID NO: 419 - DNA, 58 bp, Artificial Sequence
  Description of Artificial Sequence Synthetic oligonucleotide
ccggtttcagtgtcctagcggaatgctcgagcattccgctaggacactgaaatttttg

SEQ ID NO: 420 - DNA, 58 bp, Artificial Sequence
  Description of Artificial Sequence Synthetic oligonucleotide
ccggcatcacattggagtcacaatcctcgaggattgtgactccaatgtgatgtttttg

SEQ ID NO: 421 - DNA, 58 bp, Artificial Sequence
  Description of Artificial Sequence Synthetic oligonucleotide
ccggggacaatcagatagttcaaacctcgaggtttgaactatctgattgtcctttttg

SEQ ID NO: 422 - DNA, 58 bp, Artificial Sequence
  Description of Artificial Sequence Synthetic oligonucleotide
ccggctgttgatgacgtgttctatcctcgaggatagaacacgtcatcaacagtttttg

SEQ ID NO: 423 - DNA, 58 bp, Artificial Sequence
  Description of Artificial Sequence Synthetic oligonucleotide
ccggaggactcactgaaggagtttgctcgagcaaactccttcagtgagtccttttttg

SEQ ID NO: 424 - DNA, 58 bp, Artificial Sequence
  Description of Artificial Sequence Synthetic oligonucleotide
ccggtgaaggagtttgtgcagtttgctcgagcaaactgcacaaactccttcatttttg

SEQ ID NO: 425 - DNA, 58 bp, Artificial Sequence
  Description of Artificial Sequence Synthetic oligonucleotide
ccggttcctatgcctgattccaaatctcgagatttggaatcaggcataggaatttttg

SEQ ID NO: 426 - DNA, 58 bp, Artificial Sequence
  Description of Artificial Sequence Synthetic oligonucleotide
ccggtcctatgcctgattccaaatcctcgaggatttggaatcaggcataggatttttg

SEQ ID NO: 427 - DNA, 58 bp, Artificial Sequence
  Description of Artificial Sequence Synthetic oligonucleotide
ccggactcctccatcaccttcattcctcgaggaatgaaggtgatggaggagttttttg

SEQ ID NO: 428 - DNA, 58 bp, Artificial Sequence
  Description of Artificial Sequence Synthetic oligonucleotide
ccggagtcctggttcctctgataagctcgagcttatcagaggaaccaggacttttttg

SEQ ID NO: 429 - DNA, 58 bp, Artificial Sequence
  Description of Artificial Sequence Synthetic oligonucleotide
ccggagcagccaagcagaaagaaacctcgaggtttctttctgcttggctgcttttttg

SEQ ID NO: 430 - DNA, 58 bp, Artificial Sequence
  Description of Artificial Sequence Synthetic oligonucleotide
ccggtgtcaagtagaggacagaaagctcgagctttctgtcctctacttgacatttttg

SEQ ID NO: 431 - DNA, 58 bp, Artificial Sequence
  Description of Artificial Sequence Synthetic oligonucleotide
ccggagaggacagaaagcaagaaagctcgagctttcttgctttctgtcctcttttttg

SEQ ID NO: 432 - DNA, 58 bp, Artificial Sequence
  Description of Artificial Sequence Synthetic oligonucleotide
ccggacaaagcaaccagcacaattcctcgaggaattgtgctggttgctttgttttttg

SEQ ID NO: 433 - DNA, 58 bp, Artificial Sequence
  Description of Artificial Sequence Synthetic oligonucleotide
ccggatgggaactcctcctccatttctcgagaaatggaggaggagttcccattttttg

SEQ ID NO: 434 - DNA, 58 bp, Artificial Sequence
  Description of Artificial Sequence Synthetic oligonucleotide
ccggtgggaactcctcctccatttgctcgagcaaatggaggaggagttcccatttttg

SEQ ID NO: 435 - DNA, 58 bp, Artificial Sequence
  Description of Artificial Sequence Synthetic oligonucleotide
ccgggtccttgtgcctctctctttcctcgaggaaagagagaggcacaaggactttttg

SEQ ID NO: 436 - DNA, 58 bp, Artificial Sequence
  Description of Artificial Sequence Synthetic oligonucleotide
ccggctctctctttctctctctttcctcgaggaaagagagagaaagagagagtttttg

SEQ ID NO: 437 - DNA, 58 bp, Artificial Sequence
  Description of Artificial Sequence Synthetic oligonucleotide
ccggtctctctttcttaattgtatgctcgagcatacaattaagaaagagagatttttg

SEQ ID NO: 438 - DNA, 58 bp, Artificial Sequence
  Description of Artificial Sequence Synthetic oligonucleotide
ccgggggctgtatttattgtgaatcctcgaggattcacaataaatacagccctttttg

SEQ ID NO: 439 - DNA, 58 bp, Artificial Sequence
  Description of Artificial Sequence Synthetic oligonucleotide
ccggtttcagttcggttggctaaacctcgaggtttagccaaccgaactgaaatttttg

SEQ ID NO: 440 - DNA, 58 bp, Artificial Sequence
  Description of Artificial Sequence Synthetic oligonucleotide
ccggttcctatctttgtatgttaagctcgagcttaacatacaaagataggaatttttg

SEQ ID NO: 441 - DNA, 58 bp, Artificial Sequence
  Description of Artificial Sequence Synthetic oligonucleotide
ccggtttcgtcgttcgttctgtttcctcgaggaaacagaacgaacgacgaaatttttg

SEQ ID NO: 442 - DNA, 58 bp, Artificial Sequence
  Description of Artificial Sequence Synthetic oligonucleotide
ccggccacaaactattatacataagctcgagcttatgtataatagtttgtggtttttg

SEQ ID NO: 443 - DNA, 58 bp, Artificial Sequence
  Description of Artificial Sequence Synthetic oligonucleotide
ccggtaaatatcctgcacttgttagctcgagctaacaagtgcaggatatttatttttg

SEQ ID NO: 444 - DNA, 58 bp, Artificial Sequence
  Description of Artificial Sequence Synthetic oligonucleotide
ccggatcctgcacttgttaggaatactcgagtattcctaacaagtgcaggattttttg

SEQ ID NO: 445 - DNA, 58 bp, Artificial Sequence
  Description of Artificial Sequence Synthetic oligonucleotide
ccggtcctgcacttgttaggaatacctcgaggtattcctaacaagtgcaggatttttg

SEQ ID NO: 446 - DNA, 58 bp, Artificial Sequence
  Description of Artificial Sequence Synthetic oligonucleotide
ccggacttgttaggaatacgcatatctcgagatatgcgtattcctaacaagttttttg

SEQ ID NO: 447 - DNA, 58 bp, Artificial Sequence
  Description of Artificial Sequence Synthetic oligonucleotide
ccggcttgttaggaatacgcatatcctcgaggatatgcgtattcctaacaagtttttg

SEQ ID NO: 448 - DNA, 58 bp, Artificial Sequence
  Description of Artificial Sequence Synthetic oligonucleotide
ccggggtcaatgccaacgaacaaacctcgaggtttgttcgttggcattgacctttttg

SEQ ID NO: 449 - DNA, 58 bp, Artificial Sequence
  Description of Artificial Sequence Synthetic oligonucleotide
ccggacttgaccctcaccagcattactcgagtaatgctggtgagggtcaagttttttg

SEQ ID NO: 450 - DNA, 58 bp, Artificial Sequence
  Description of Artificial Sequence Synthetic oligonucleotide
ccgggctcagcaagttggtagtttcctcgaggaaactaccaacttgctgagctttttg

SEQ ID NO: 451 - DNA, 58 bp, Artificial Sequence
  Description of Artificial Sequence Synthetic oligonucleotide
ccggccgtcccgtcaaacaagtttcctcgaggaaacttgtttgacgggacggtttttg

SEQ ID NO: 452 - DNA, 58 bp, Artificial Sequence
  Description of Artificial Sequence Synthetic oligonucleotide
ccgggtcaaacaagtttcttcttagctcgagctaagaagaaacttgtttgactttttg

SEQ ID NO: 453 - DNA, 58 bp, Artificial Sequence
  Description of Artificial Sequence Synthetic oligonucleotide
ccggctctgcactgagatacataagctcgagcttatgtatctcagtgcagagtttttg

SEQ ID NO: 454 - DNA, 58 bp, Artificial Sequence
  Description of Artificial Sequence Synthetic oligonucleotide
ccgggcactgagatacataagaaacctcgaggtttcttatgtatctcagtgctttttg

SEQ ID NO: 455 - DNA, 58 bp, Artificial Sequence
  Description of Artificial Sequence Synthetic oligonucleotide
ccggtaagaaacaagggtagtttacctcgaggtaaactacccttgtttcttatttttg

SEQ ID NO: 456 - DNA, 58 bp, Artificial Sequence
  Description of Artificial Sequence Synthetic oligonucleotide
ccgggcttctgttataccatctttgctcgagcaaagatggtataacagaagctttttg

SEQ ID NO: 457 - DNA, 58 bp, Artificial Sequence
  Description of Artificial Sequence Synthetic oligonucleotide
ccggctccggcttctccattgaatgctcgagcattcaatggagaagccggagtttttg

SEQ ID NO: 458 - DNA, 58 bp, Artificial Sequence
  Description of Artificial Sequence Synthetic oligonucleotide
ccggcttctccattgaatggctaatctcgagattagccattcaatggagaagtttttg

SEQ ID NO: 459 - DNA, 58 bp, Artificial Sequence
  Description of Artificial Sequence Synthetic oligonucleotide
ccggttctccattgaatggctaatgctcgagcattagccattcaatggagaatttttg

SEQ ID NO: 460 - DNA, 58 bp, Artificial Sequence
  Description of Artificial Sequence Synthetic oligonucleotide
ccggattgaatggctaatgtgtatgctcgagcatacacattagccattcaattttttg

SEQ ID NO: 461 - DNA, 58 bp, Artificial Sequence
  Description of Artificial Sequence Synthetic oligonucleotide
ccggcagtcctggttcctctgataactcgagttatcagaggaaccaggactgtttttg

SEQ ID NO: 462 - DNA, 58 bp, Artificial Sequence
  Description of Artificial Sequence Synthetic oligonucleotide
ccggctgtaatgtctttactctttactcgagtaaagagtaaagacattacagtttttg

SEQ ID NO: 463 - DNA, 58 bp, Artificial Sequence
  Description of Artificial Sequence Synthetic oligonucleotide
ccggttgagcaacttgggaacaattctcgagaattgttcccaagttgctcaatttttg

SEQ ID NO: 464 - DNA, 58 bp, Artificial Sequence
  Description of Artificial Sequence Synthetic oligonucleotide
ccggtgagcaacttgggaacaatttctcgagaaattgttcccaagttgctcatttttg

SEQ ID NO: 465 - DNA, 58 bp, Artificial Sequence
  Description of Artificial Sequence Synthetic oligonucleotide
ccgggagcaacttgggaacaatttgctcgagcaaattgttcccaagttgctctttttg

SEQ ID NO: 466 - DNA, 58 bp, Artificial Sequence
  Description of Artificial Sequence Synthetic oligonucleotide
ccggctctttctctctctttcttaactcgagttaagaaagagagagaaagagtttttg

SEQ ID NO: 467 - DNA, 58 bp, Artificial Sequence
  Description of Artificial Sequence Synthetic oligonucleotide
ccggtctttctctctctttcttaatctcgagattaagaaagagagagaaagatttttg

SEQ ID NO: 468 - DNA, 58 bp, Artificial Sequence
  Description of Artificial Sequence Synthetic oligonucleotide
ccggtttctctctctttcttaattgctcgagcaattaagaaagagagagaaatttttg

SEQ ID NO: 469 - DNA, 58 bp, Artificial Sequence
  Description of Artificial Sequence Synthetic oligonucleotide
ccggggtgggctatgggctgtatttctcgagaaatacagcccatagcccacctttttg

SEQ ID NO: 470 - DNA, 58 bp, Artificial Sequence
  Description of Artificial Sequence Synthetic oligonucleotide
ccggggctatgggctgtatttattgctcgagcaataaatacagcccatagcctttttg

SEQ ID NO: 471 - DNA, 58 bp, Artificial Sequence
  Description of Artificial Sequence Synthetic oligonucleotide
ccgggcggtcaaagtgcaatgatttctcgagaaatcattgcactttgaccgctttttg

SEQ ID NO: 472 - DNA, 58 bp, Artificial Sequence
  Description of Artificial Sequence Synthetic oligonucleotide
ccggctgaaggaaacgttcacatttctcgagaaatgtgaacgtttccttcagtttttg

SEQ ID NO: 473 - DNA, 58 bp, Artificial Sequence
  Description of Artificial Sequence Synthetic oligonucleotide
ccggaccctggtgcttcttacattactcgagtaatgtaagaagcaccagggttttttg

SEQ ID NO: 474 - DNA, 58 bp, Artificial Sequence
  Description of Artificial Sequence Synthetic oligonucleotide
ccggccctggtgcttcttacattatctcgagataatgtaagaagcaccagggtttttg

SEQ ID NO: 475 - DNA, 58 bp, Artificial Sequence
  Description of Artificial Sequence Synthetic oligonucleotide
ccggggtgcttcttacattataaagctcgagctttataatgtaagaagcacctttttg

SEQ ID NO: 476 - DNA, 58 bp, Artificial Sequence
  Description of Artificial Sequence Synthetic oligonucleotide
ccggttaaagaacccacaaactattctcgagaatagtttgtgggttctttaatttttg

SEQ ID NO: 477 - DNA, 58 bp, Artificial Sequence
  Description of Artificial Sequence Synthetic oligonucleotide
ccggtaaagaacccacaaactattactcgagtaatagtttgtgggttctttatttttg

SEQ ID NO: 478 - DNA, 58 bp, Artificial Sequence
  Description of Artificial Sequence Synthetic oligonucleotide
ccggagaacccacaaactattatacctcgaggtataatagtttgtgggttcttttttg

SEQ ID NO: 479 - DNA, 58 bp, Artificial Sequence
  Description of Artificial Sequence Synthetic oligonucleotide
ccggcccacaaactattatacataactcgagttatgtataatagtttgtgggtttttg

SEQ ID NO: 480 - DNA, 58 bp, Artificial Sequence
  Description of Artificial Sequence Synthetic oligonucleotide
ccggatcccttctttgttgagtttactcgagtaaactcaacaaagaagggattttttg

SEQ ID NO: 481 - DNA, 58 bp, Artificial Sequence
  Description of Artificial Sequence Synthetic oligonucleotide
ccggtcccttctttgttgagtttaactcgagttaaactcaacaaagaagggatttttg

SEQ ID NO: 482 - DNA, 58 bp, Artificial Sequence
  Description of Artificial Sequence Synthetic oligonucleotide
ccggcccttctttgttgagtttaacctcgaggttaaactcaacaaagaagggtttttg

SEQ ID NO: 483 - DNA, 58 bp, Artificial Sequence
  Description of Artificial Sequence Synthetic oligonucleotide
ccggcttgaccctcaccagcattatctcgagataatgctggtgagggtcaagtttttg

SEQ ID NO: 484 - DNA, 58 bp, Artificial Sequence
  Description of Artificial Sequence Synthetic oligonucleotide
ccggttgaccctcaccagcattatcctcgaggataatgctggtgagggtcaatttttg

SEQ ID NO: 485 - DNA, 58 bp, Artificial Sequence
  Description of Artificial Sequence Synthetic oligonucleotide
ccggagccctgtcgactgccttatactcgagtataaggcagtcgacagggcttttttg

SEQ ID NO: 486 - DNA, 58 bp, Artificial Sequence
  Description of Artificial Sequence Synthetic oligonucleotide
ccggaggctaagaaacgcagtatatctcgagatatactgcgtttcttagccttttttg

SEQ ID NO: 487 - DNA, 58 bp, Artificial Sequence
  Description of Artificial Sequence Synthetic oligonucleotide
ccgggctaagaaacgcagtatatacctcgaggtatatactgcgtttcttagctttttg

SEQ ID NO: 488 - DNA, 58 bp, Artificial Sequence
  Description of Artificial Sequence Synthetic oligonucleotide
ccgggtatatacgagtatctctatactcgagtatagagatactcgtatatactttttg

SEQ ID NO: 489 - DNA, 58 bp, Artificial Sequence
  Description of Artificial Sequence Synthetic oligonucleotide
ccggtacgagtatctctatatatagctcgagctatatatagagatactcgtatttttg

SEQ ID NO: 490 - DNA, 58 bp, Artificial Sequence
  Description of Artificial Sequence Synthetic oligonucleotide
ccggtctctgcactgagatacataactcgagttatgtatctcagtgcagagatttttg

SEQ ID NO: 491 - DNA, 58 bp, Artificial Sequence
  Description of Artificial Sequence Synthetic oligonucleotide
ccggataagaaacaagggtagtttactcgagtaaactacccttgtttcttattttttg

SEQ ID NO: 492 - DNA, 58 bp, Artificial Sequence
  Description of Artificial Sequence Synthetic oligonucleotide
ccggccgtcactgcgcttctgttatctcgagataacagaagcgcagtgacggtttttg

SEQ ID NO: 493 - DNA, 58 bp, Artificial Sequence
  Description of Artificial Sequence Synthetic oligonucleotide
ccggcgtcactgcgcttctgttatactcgagtataacagaagcgcagtgacgtttttg

SEQ ID NO: 494 - DNA, 58 bp, Artificial Sequence
  Description of Artificial Sequence Synthetic oligonucleotide
ccgggtcactgcgcttctgttatacctcgaggtataacagaagcgcagtgactttttg

SEQ ID NO: 495 - DNA, 58 bp, Artificial Sequence
  Description of Artificial Sequence Synthetic oligonucleotide
ccggtggctaatgtgtatgtgaaatctcgagatttcacatacacattagccatttttg

SEQ ID NO: 496 - DNA, 58 bp, Artificial Sequence
  Description of Artificial Sequence Synthetic oligonucleotide
ccggggctaatgtgtatgtgaaatactcgagtatttcacatacacattagcctttttg

SEQ ID NO: 497 - DNA, 58 bp, Artificial Sequence
  Description of Artificial Sequence Synthetic oligonucleotide
ccggctttctctctctttcttaattctcgagaattaagaaagagagagaaagtttttg

SEQ ID NO: 498 - DNA, 58 bp, Artificial Sequence
  Description of Artificial Sequence Synthetic oligonucleotide
ccgggtgggctatgggctgtatttactcgagtaaatacagcccatagcccactttttg

SEQ ID NO: 499 - DNA, 58 bp, Artificial Sequence
  Description of Artificial Sequence Synthetic oligonucleotide
ccggtgggctatgggctgtatttatctcgagataaatacagcccatagcccatttttg

SEQ ID NO: 500 - DNA, 58 bp, Artificial Sequence
  Description of Artificial Sequence Synthetic oligonucleotide
ccgggggctatgggctgtatttattctcgagaataaatacagcccatagccctttttg

SEQ ID NO: 501 - DNA, 58 bp, Artificial Sequence
  Description of Artificial Sequence Synthetic oligonucleotide
ccggcctggtgcttcttacattatactcgagtataatgtaagaagcaccaggtttttg

SEQ ID NO: 502 - DNA, 58 bp, Artificial Sequence
  Description of Artificial Sequence Synthetic oligonucleotide
ccggctggtgcttcttacattataactcgagttataatgtaagaagcaccagtttttg

SEQ ID NO: 503 - DNA, 58 bp, Artificial Sequence
  Description of Artificial Sequence Synthetic oligonucleotide
ccggtggtgcttcttacattataaactcgagtttataatgtaagaagcaccatttttg

SEQ ID NO: 504 - DNA, 58 bp, Artificial Sequence
  Description of Artificial Sequence Synthetic oligonucleotide
ccggggctaagaaacgcagtatatactcgagtatatactgcgtttcttagcctttttg

SEQ ID NO: 505 - DNA, 58 bp, Artificial Sequence
  Description of Artificial Sequence Synthetic oligonucleotide
ccgggctaatgtgtatgtgaaataactcgagttatttcacatacacattagctttttg

SEQ ID NO: 506 - DNA, 58 bp, Artificial Sequence
  Description of Artificial Sequence Synthetic oligonucleotide
aattcaaaaacctgttgatgacgtgttctatctcgagatagaacacgtcatcaacagg

SEQ ID NO: 507 - DNA, 58 bp, Artificial Sequence
  Description of Artificial Sequence Synthetic oligonucleotide
aattcaaaaacacatcacattggagtcacaactcgagttgtgactccaatgtgatgtg

SEQ ID NO: 508 - DNA, 58 bp, Artificial Sequence
  Description of Artificial Sequence Synthetic oligonucleotide
aattcaaaaaccggacaatcagatagttcaactcgagttgaactatctgattgtccgg

SEQ ID NO: 509 - DNA, 58 bp, Artificial Sequence
  Description of Artificial Sequence Synthetic oligonucleotide
aattcaaaaaggaatccggacaatcagatagctcgagctatctgattgtccggattcc

SEQ ID NO: 510 - DNA, 58 bp, Artificial Sequence
  Description of Artificial Sequence Synthetic oligonucleotide
aattcaaaaacccaacgggcacttaagtttcctcgaggaaacttaagtgcccgttggg

SEQ ID NO: 511 - DNA, 58 bp, Artificial Sequence
  Description of Artificial Sequence Synthetic oligonucleotide
aattcaaaaaacattggagtcacaatcaaagctcgagctttgattgtgactccaatgt

SEQ ID NO: 512 - DNA, 58 bp, Artificial Sequence
  Description of Artificial Sequence Synthetic oligonucleotide
aattcaaaaaatctttatctggcttactttgctcgagcaaagtaagccagataaagat

SEQ ID NO: 513 - DNA, 58 bp, Artificial Sequence
  Description of Artificial Sequence Synthetic oligonucleotide
aattcaaaaataggctaagaaacgcagtatactcgagtatactgcgtttcttagccta

SEQ ID NO: 514 - DNA, 58 bp, Artificial Sequence
  Description of Artificial Sequence Synthetic oligonucleotide
aattcaaaaatggacctggtcatggtgatttctcgagaaatcaccatgaccaggtcca

SEQ ID NO: 515 - DNA, 58 bp, Artificial Sequence
  Description of Artificial Sequence Synthetic oligonucleotide
aattcaaaaaggatgagttccacccgttcatctcgagatgaacgggtggaactcatcc

SEQ ID NO: 516 - DNA, 58 bp, Artificial Sequence
  Description of Artificial Sequence Synthetic oligonucleotide
aattcaaaaatttctcctacacctggttcaactcgagttgaaccaggtgtaggagaaa

SEQ ID NO: 517 - DNA, 58 bp, Artificial Sequence
  Description of Artificial Sequence Synthetic oligonucleotide
aattcaaaaattctcctacacctggttcaacctcgaggttgaaccaggtgtaggagaa

SEQ ID NO: 518 - DNA, 58 bp, Artificial Sequence
  Description of Artificial Sequence Synthetic oligonucleotide
aattcaaaaagcggaagcgcaagtacttcaactcgagttgaagtacttgcgcttccgc

SEQ ID NO: 519 - DNA, 58 bp, Artificial Sequence
  Description of Artificial Sequence Synthetic oligonucleotide
aattcaaaaacggaagcgcaagtacttcaagctcgagcttgaagtacttgcgcttccg

SEQ ID NO: 520 - DNA, 58 bp, Artificial Sequence
  Description of Artificial Sequence Synthetic oligonucleotide
aattcaaaaaggaagcgcaagtacttcaagactcgagtcttgaagtacttgcgcttcc

SEQ ID NO: 521 - DNA, 58 bp, Artificial Sequence
  Description of Artificial Sequence Synthetic oligonucleotide
aattcaaaaagaagcgcaagtacttcaagaactcgagttcttgaagtacttgcgcttc

SEQ ID NO: 522 - DNA, 58 bp, Artificial Sequence
  Description of Artificial Sequence Synthetic oligonucleotide
aattcaaaaaagcccgagatcaagcagaagtctcgagacttctgcttgatctcgggct

SEQ ID NO: 523 - DNA, 58 bp, Artificial Sequence
  Description of Artificial Sequence Synthetic oligonucleotide
aattcaaaaactggacctggtcatggtgattctcgagaatcaccatgaccaggtccag

SEQ ID NO: 524 - DNA, 58 bp, Artificial Sequence
  Description of Artificial Sequence Synthetic oligonucleotide
aattcaaaaatgcgtccagccacatcacattctcgagaatgtgatgtggctggacgca

SEQ ID NO: 525 - DNA, 58 bp, Artificial Sequence
  Description of Artificial Sequence Synthetic oligonucleotide
aattcaaaaaacatcacattggagtcacaatctcgagattgtgactccaatgtgatgt

SEQ ID NO: 526 - DNA, 58 bp, Artificial Sequence
  Description of Artificial Sequence Synthetic oligonucleotide
aattcaaaaaatcacattggagtcacaatcactcgagtgattgtgactccaatgtgat

SEQ ID NO: 527 - DNA, 58 bp, Artificial Sequence
  Description of Artificial Sequence Synthetic oligonucleotide
aattcaaaaacattggagtcacaatcaaagactcgagtctttgattgtgactccaatg

SEQ ID NO: 528 - DNA, 58 bp, Artificial Sequence
  Description of Artificial Sequence Synthetic oligonucleotide
aattcaaaaattggagtcacaatcaaagaacctcgaggttctttgattgtgactccaa

SEQ ID NO: 529 - DNA, 58 bp, Artificial Sequence
  Description of Artificial Sequence Synthetic oligonucleotide
aattcaaaaatggagtcacaatcaaagaactctcgagagttctttgattgtgactcca

SEQ ID NO: 530 - DNA, 58 bp, Artificial Sequence
  Description of Artificial Sequence Synthetic oligonucleotide
aattcaaaaaacaatcaaagaactggatcttctcgagaagatccagttctttgattgt

SEQ ID NO: 531 - DNA, 58 bp, Artificial Sequence
  Description of Artificial Sequence Synthetic oligonucleotide
aattcaaaaatggatctttatctggcttactctcgagagtaagccagataaagatcca

SEQ ID NO: 532 - DNA, 58 bp, Artificial Sequence
  Description of Artificial Sequence Synthetic oligonucleotide
aattcaaaaatctttatctggcttactttgtctcgagacaaagtaagccagataaaga

SEQ ID NO: 533 - DNA, 58 bp, Artificial Sequence
  Description of Artificial Sequence Synthetic oligonucleotide
aattcaaaaactttatctggcttactttgtcctcgaggacaaagtaagccagataaag

SEQ ID NO: 534 - DNA, 58 bp, Artificial Sequence
  Description of Artificial Sequence Synthetic oligonucleotide
aattcaaaaaacactccggaatccggacaatctcgagattgtccggattccggagtgt

SEQ ID NO: 535 - DNA, 58 bp, Artificial Sequence
  Description of Artificial Sequence Synthetic oligonucleotide
aattcaaaaaactccggaatccggacaatcactcgagtgattgtccggattccggagt

SEQ ID NO: 536 - DNA, 58 bp, Artificial Sequence
  Description of Artificial Sequence Synthetic oligonucleotide
aattcaaaaaccggaatccggacaatcagatctcgagatctgattgtccggattccgg

SEQ ID NO: 537 - DNA, 58 bp, Artificial Sequence
  Description of Artificial Sequence Synthetic oligonucleotide
aattcaaaaacggaatccggacaatcagatactcgagtatctgattgtccggattccg

SEQ ID NO: 538 - DNA, 58 bp, Artificial Sequence
  Description of Artificial Sequence Synthetic oligonucleotide
aattcaaaaagaatccggacaatcagatagtctcgagactatctgattgtccggattc

SEQ ID NO: 539 - DNA, 58 bp, Artificial Sequence
  Description of Artificial Sequence Synthetic oligonucleotide
aattcaaaaaatccggacaatcagatagttcctcgaggaactatctgattgtccggat

SEQ ID NO: 540 - DNA, 58 bp, Artificial Sequence
  Description of Artificial Sequence Synthetic oligonucleotide
aattcaaaaatccggacaatcagatagttcactcgagtgaactatctgattgtccgga

SEQ ID NO: 541 - DNA, 58 bp, Artificial Sequence
  Description of Artificial Sequence Synthetic oligonucleotide
aattcaaaaaagcaaggagatgcggacatcactcgagtgatgtccgcatctccttgct

SEQ ID NO: 542 - DNA, 58 bp, Artificial Sequence
  Description of Artificial Sequence Synthetic oligonucleotide
aattcaaaaagcaaggagatgcggacatcaactcgagttgatgtccgcatctccttgc

SEQ ID NO: 543 - DNA, 58 bp, Artificial Sequence
  Description of Artificial Sequence Synthetic oligonucleotide
aattcaaaaaacttaagtttccaggactgttctcgagaacagtcctggaaacttaagt

SEQ ID NO: 544 - DNA, 58 bp, Artificial Sequence
  Description of Artificial Sequence Synthetic oligonucleotide
aattcaaaaacttaagtttccaggactgtttctcgagaaacagtcctggaaacttaag

SEQ ID NO: 545 - DNA, 58 bp, Artificial Sequence
  Description of Artificial Sequence Synthetic oligonucleotide
aattcaaaaatgacggagctggtgagagtatctcgagatactctcaccagctccgtca

SEQ ID NO: 546 - DNA, 58 bp, Artificial Sequence
  Description of Artificial Sequence Synthetic oligonucleotide
aattcaaaaaacggagctggtgagagtatcactcgagtgatactctcaccagctccgt

SEQ ID NO: 547 - DNA, 58 bp, Artificial Sequence
  Description of Artificial Sequence Synthetic oligonucleotide
aattcaaaaatggagagtcccagctactacactcgagtgtagtagctgggactctcca

SEQ ID NO: 548 - DNA, 58 bp, Artificial Sequence
  Description of Artificial Sequence Synthetic oligonucleotide
aattcaaaaaggagagtcccagctactacaactcgagttgtagtagctgggactctcc

SEQ ID NO: 549 - DNA, 58 bp, Artificial Sequence
  Description of Artificial Sequence Synthetic oligonucleotide
aattcaaaaagagagtcccagctactacaacctcgaggttgtagtagctgggactctc

SEQ ID NO: 550 - DNA, 58 bp, Artificial Sequence
  Description of Artificial Sequence Synthetic oligonucleotide
aattcaaaaaagagtcccagctactacaacactcgagtgttgtagtagctgggactct

SEQ ID NO: 551 - DNA, 58 bp, Artificial Sequence
  Description of Artificial Sequence Synthetic oligonucleotide
aattcaaaaagagtcccagctactacaacatctcgagatgttgtagtagctgggactc

SEQ ID NO: 552 - DNA, 58 bp, Artificial Sequence
  Description of Artificial Sequence Synthetic oligonucleotide
aattcaaaaaagtcccagctactacaacatcctcgaggatgttgtagtagctgggact

SEQ ID NO: 553 - DNA, 58 bp, Artificial Sequence
  Description of Artificial Sequence Synthetic oligonucleotide
aattcaaaaagtcccagctactacaacatcactcgagtgatgttgtagtagctgggac

SEQ ID NO: 554 - DNA, 58 bp, Artificial Sequence
  Description of Artificial Sequence Synthetic oligonucleotide
aattcaaaaatcccagctactacaacatcaactcgagttgatgttgtagtagctggga

SEQ ID NO: 555 - DNA, 58 bp, Artificial Sequence
  Description of Artificial Sequence Synthetic oligonucleotide
aattcaaaaacccagctactacaacatcaacctcgaggttgatgttgtagtagctggg

SEQ ID NO: 556 - DNA, 58 bp, Artificial Sequence
  Description of Artificial Sequence Synthetic oligonucleotide
aattcaaaaagtccatcgatgacagtgagatctcgagatctcactgtcatcgatggac

SEQ ID NO: 557 - DNA, 58 bp, Artificial Sequence
  Description of Artificial Sequence Synthetic oligonucleotide
aattcaaaaatgagatggagagccctgttgactcgagtcaacagggctctccatctca

SEQ ID NO: 558 - DNA, 58 bp, Artificial Sequence
  Description of Artificial Sequence Synthetic oligonucleotide
aattcaaaaagagatggagagccctgttgatctcgagatcaacagggctctccatctc

SEQ ID NO: 559 - DNA, 58 bp, Artificial Sequence
  Description of Artificial Sequence Synthetic oligonucleotide
aattcaaaaaagatggagagccctgttgatgctcgagcatcaacagggctctccatct

SEQ ID NO: 560 - DNA, 58 bp, Artificial Sequence
  Description of Artificial Sequence Synthetic oligonucleotide
aattcaaaaagatggagagccctgttgatgactcgagtcatcaacagggctctccatc

SEQ ID NO: 561 - DNA, 58 bp, Artificial Sequence
  Description of Artificial Sequence Synthetic oligonucleotide
aattcaaaaagccctgttgatgacgtgttctctcgagagaacacgtcatcaacagggc

SEQ ID NO: 562 - DNA, 58 bp, Artificial Sequence
  Description of Artificial Sequence Synthetic oligonucleotide
aattcaaaaaccctgttgatgacgtgttctactcgagtagaacacgtcatcaacaggg

SEQ ID NO: 563 - DNA, 58 bp, Artificial Sequence
  Description of Artificial Sequence Synthetic oligonucleotide
aattcaaaaattctctaaagaagtcaggaaactcgagtttcctgacttctttagagaa

SEQ ID NO: 564 - DNA, 58 bp, Artificial Sequence
  Description of Artificial Sequence Synthetic oligonucleotide
aattcaaaaaagtcaggaaagctggacttctctcgagagaagtccagctttcctgact

SEQ ID NO: 565 - DNA, 58 bp, Artificial Sequence
  Description of Artificial Sequence Synthetic oligonucleotide
aattcaaaaacaggactcactgaaggagtttctcgagaaactccttcagtgagtcctg

SEQ ID NO: 566 - DNA, 58 bp, Artificial Sequence
  Description of Artificial Sequence Synthetic oligonucleotide
aattcaaaaaggactcactgaaggagtttgtctcgagacaaactccttcagtgagtcc

SEQ ID NO: 567 - DNA, 58 bp, Artificial Sequence
  Description of Artificial Sequence Synthetic oligonucleotide
aattcaaaaagactcactgaaggagtttgtgctcgagcacaaactccttcagtgagtc

SEQ ID NO: 568 - DNA, 58 bp, Artificial Sequence
  Description of Artificial Sequence Synthetic oligonucleotide
aattcaaaaactgaaggagtttgtgcagtttctcgagaaactgcacaaactccttcag

SEQ ID NO: 569 - DNA, 58 bp, Artificial Sequence
  Description of Artificial Sequence Synthetic oligonucleotide
aattcaaaaagaaggagtttgtgcagtttgtctcgagacaaactgcacaaactccttc

SEQ ID NO: 570 - DNA, 58 bp, Artificial Sequence
  Description of Artificial Sequence Synthetic oligonucleotide
aattcaaaaacttcctatgcctgattccaaactcgagtttggaatcaggcataggaag

SEQ ID NO: 571 - DNA, 58 bp, Artificial Sequence
  Description of Artificial Sequence Synthetic oligonucleotide
aattcaaaaacctatgcctgattccaaatccctcgagggatttggaatcaggcatagg

SEQ ID NO: 572 - DNA, 58 bp, Artificial Sequence
  Description of Artificial Sequence Synthetic oligonucleotide
aattcaaaaatgactcctccatcaccttcatctcgagatgaaggtgatggaggagtca

SEQ ID NO: 573 - DNA, 58 bp, Artificial Sequence
  Description of Artificial Sequence Synthetic oligonucleotide
aattcaaaaagggacggcaactttctgaacactcgagtgttcagaaagttgccgtccc

SEQ ID NO: 574 - DNA, 58 bp, Artificial Sequence
  Description of Artificial Sequence Synthetic oligonucleotide
aattcaaaaaggacggcaactttctgaacatctcgagatgttcagaaagttgccgtcc

SEQ ID NO: 575 - DNA, 58 bp, Artificial Sequence
  Description of Artificial Sequence Synthetic oligonucleotide
aattcaaaaagacggcaactttctgaacatcctcgaggatgttcagaaagttgccgtc

SEQ ID NO: 576 - DNA, 58 bp, Artificial Sequence
  Description of Artificial Sequence Synthetic oligonucleotide
aattcaaaaactcagtcctggttcctctgatctcgagatcagaggaaccaggactgag

SEQ ID NO: 577 - DNA, 58 bp, Artificial Sequence
  Description of Artificial Sequence Synthetic oligonucleotide
aattcaaaaatcagtcctggttcctctgatactcgagtatcagaggaaccaggactga

SEQ ID NO: 578 - DNA, 58 bp, Artificial Sequence
  Description of Artificial Sequence Synthetic oligonucleotide
aattcaaaaacctggttcctctgataagatcctcgaggatcttatcagaggaaccagg

SEQ ID NO: 579 - DNA, 58 bp, Artificial Sequence
  Description of Artificial Sequence Synthetic oligonucleotide
aattcaaaaatcctctgataagatcgacaaactcgagtttgtcgatcttatcagagga

SEQ ID NO: 580 - DNA, 58 bp, Artificial Sequence
  Description of Artificial Sequence Synthetic oligonucleotide
aattcaaaaaagaagaagaggttcctcgaaactcgagtttcgaggaacctcttcttct

SEQ ID NO: 581 - DNA, 58 bp, Artificial Sequence
  Description of Artificial Sequence Synthetic oligonucleotide
aattcaaaaaaccagcagccaagcagaaagactcgagtctttctgcttggctgctggt

SEQ ID NO: 582 - DNA, 58 bp, Artificial Sequence
  Description of Artificial Sequence Synthetic oligonucleotide
aattcaaaaagcagccaagcagaaagaaacactcgagtgtttctttctgcttggctgc

SEQ ID NO: 583 - DNA, 58 bp, Artificial Sequence
  Description of Artificial Sequence Synthetic oligonucleotide
aattcaaaaacagccaagcagaaagaaacacctcgaggtgtttctttctgcttggctg

SEQ ID NO: 584 - DNA, 58 bp, Artificial Sequence
  Description of Artificial Sequence Synthetic oligonucleotide
aattcaaaaaacgcgacatggactctgtcaactcgagttgacagagtccatgtcgcgt

SEQ ID NO: 585 - DNA, 58 bp, Artificial Sequence
  Description of Artificial Sequence Synthetic oligonucleotide
aattcaaaaagcgacatggactctgtcaagtctcgagacttgacagagtccatgtcgc

SEQ ID NO: 586 - DNA, 58 bp, Artificial Sequence
  Description of Artificial Sequence Synthetic oligonucleotide
aattcaaaaacgacatggactctgtcaagtactcgagtacttgacagagtccatgtcg

SEQ ID NO: 587 - DNA, 58 bp, Artificial Sequence
  Description of Artificial Sequence Synthetic oligonucleotide
aattcaaaaagacatggactctgtcaagtagctcgagctacttgacagagtccatgtc

SEQ ID NO: 588 - DNA, 58 bp, Artificial Sequence
  Description of Artificial Sequence Synthetic oligonucleotide
aattcaaaaaacatggactctgtcaagtagactcgagtctacttgacagagtccatgt

SEQ ID NO: 589 - DNA, 58 bp, Artificial Sequence
  Description of Artificial Sequence Synthetic oligonucleotide
aattcaaaaagtagaggacagaaagcaagaactcgagttcttgctttctgtcctctac

SEQ ID NO: 590 - DNA, 58 bp, Artificial Sequence
  Description of Artificial Sequence Synthetic oligonucleotide
aattcaaaaaagcaagaaaggatgcagaactctcgagagttctgcatcctttcttgct

SEQ ID NO: 591 - DNA, 58 bp, Artificial Sequence
  Description of Artificial Sequence Synthetic oligonucleotide
aattcaaaaaagcaaccagcacaattctgaactcgagttcagaattgtgctggttgct

SEQ ID NO: 592 - DNA, 58 bp, Artificial Sequence
  Description of Artificial Sequence Synthetic oligonucleotide
aattcaaaaagagggccagcttgtaaagatgctcgagcatctttacaagctggccctc

SEQ ID NO: 593 - DNA, 58 bp, Artificial Sequence
  Description of Artificial Sequence Synthetic oligonucleotide
aattcaaaaaagggccagcttgtaaagatgactcgagtcatctttacaagctggccct

SEQ ID NO: 594 - DNA, 58 bp, Artificial Sequence
  Description of Artificial Sequence Synthetic oligonucleotide
aattcaaaaagggccagcttgtaaagatgatctcgagatcatctttacaagctggccc

SEQ ID NO: 595 - DNA, 58 bp, Artificial Sequence
  Description of Artificial Sequence Synthetic oligonucleotide
aattcaaaaaggccagcttgtaaagatgatgctcgagcatcatctttacaagctggcc

SEQ ID NO: 596 - DNA, 58 bp, Artificial Sequence
  Description of Artificial Sequence Synthetic oligonucleotide
aattcaaaaagctagttgcgtgtcccagaatctcgagattctgggacacgcaactagc

SEQ ID NO: 597 - DNA, 58 bp, Artificial Sequence
  Description of Artificial Sequence Synthetic oligonucleotide
aattcaaaaatcccagaatggagggtgttctctcgagagaacaccctccattctggga

SEQ ID NO: 598 - DNA, 58 bp, Artificial Sequence
  Description of Artificial Sequence Synthetic oligonucleotide
aattcaaaaactctgtacagtccgtaggaaactcgagtttcctacggactgtacagag

SEQ ID NO: 599 - DNA, 58 bp, Artificial Sequence
  Description of Artificial Sequence Synthetic oligonucleotide
aattcaaaaaagtacttcaagaagcatgaaactcgagtttcatgcttcttgaagtact

SEQ ID NO: 600 - DNA, 58 bp, Artificial Sequence
  Description of Artificial Sequence Synthetic oligonucleotide
aattcaaaaatcacattggagtcacaatcaactcgagttgattgtgactccaatgtga

SEQ ID NO: 601 - DNA, 58 bp, Artificial Sequence
  Description of Artificial Sequence Synthetic oligonucleotide
aattcaaaaacacattggagtcacaatcaaactcgagtttgattgtgactccaatgtg

SEQ ID NO: 602 - DNA, 58 bp, Artificial Sequence
  Description of Artificial Sequence Synthetic oligonucleotide
aattcaaaaaattggagtcacaatcaaagaactcgagttctttgattgtgactccaat

SEQ ID NO: 603 - DNA, 58 bp, Artificial Sequence
  Description of Artificial Sequence Synthetic oligonucleotide
aattcaaaaaggatctttatctggcttacttctcgagaagtaagccagataaagatcc

SEQ ID NO: 604 - DNA, 58 bp, Artificial Sequence
  Description of Artificial Sequence Synthetic oligonucleotide
aattcaaaaagatctttatctggcttactttctcgagaaagtaagccagataaagatc

SEQ ID NO: 605 - DNA, 58 bp, Artificial Sequence
  Description of Artificial Sequence Synthetic oligonucleotide
aattcaaaaacggacaatcagatagttcaaactcgagtttgaactatctgattgtccg

SEQ ID NO: 606 - DNA, 58 bp, Artificial Sequence
  Description of Artificial Sequence Synthetic oligonucleotide
aattcaaaaacaaggagatgcggacatcaaactcgagtttgatgtccgcatctccttg

SEQ ID NO: 607 - DNA, 58 bp, Artificial Sequence
  Description of Artificial Sequence Synthetic oligonucleotide
aattcaaaaatgcccaacgggcacttaagttctcgagaacttaagtgcccgttgggca

SEQ ID NO: 608 - DNA, 58 bp, Artificial Sequence
  Description of Artificial Sequence Synthetic oligonucleotide
aattcaaaaagcccaacgggcacttaagtttctcgagaaacttaagtgcccgttgggc

SEQ ID NO: 609 - DNA, 58 bp, Artificial Sequence
  Description of Artificial Sequence Synthetic oligonucleotide
aattcaaaaaagcagtcgagcccgtatttcactcgagtgaaatacgggctcgactgct

SEQ ID NO: 610 - DNA, 58 bp, Artificial Sequence
  Description of Artificial Sequence Synthetic oligonucleotide
aattcaaaaagactcctccatcaccttcattctcgagaatgaaggtgatggaggagtc

SEQ ID NO: 611 - DNA, 58 bp, Artificial Sequence
  Description of Artificial Sequence Synthetic oligonucleotide
aattcaaaaagtcctggttcctctgataagactcgagtcttatcagaggaaccaggac

SEQ ID NO: 612 - DNA, 58 bp, Artificial Sequence
  Description of Artificial Sequence Synthetic oligonucleotide
aattcaaaaatcctggttcctctgataagatctcgagatcttatcagaggaaccagga

SEQ ID NO: 613 - DNA, 58 bp, Artificial Sequence
  Description of Artificial Sequence Synthetic oligonucleotide
aattcaaaaaccagcagccaagcagaaagaactcgagttctttctgcttggctgctgg

SEQ ID NO: 614 - DNA, 58 bp, Artificial Sequence
  Description of Artificial Sequence Synthetic oligonucleotide
aattcaaaaacagcagccaagcagaaagaaactcgagtttctttctgcttggctgctg

SEQ ID NO: 615 - DNA, 58 bp, Artificial Sequence
  Description of Artificial Sequence Synthetic oligonucleotide
aattcaaaaatagaggacagaaagcaagaaactcgagtttcttgctttctgtcctcta

SEQ ID NO: 616 - DNA, 58 bp, Artificial Sequence
  Description of Artificial Sequence Synthetic oligonucleotide
aattcaaaaacaaagcaaccagcacaattctctcgagagaattgtgctggttgctttg

SEQ ID NO: 617 - DNA, 58 bp, Artificial Sequence
  Description of Artificial Sequence Synthetic oligonucleotide
aattcaaaaatggagggccagcttgtaaagactcgagtctttacaagctggccctcca

SEQ ID NO: 618 - DNA, 58 bp, Artificial Sequence
  Description of Artificial Sequence Synthetic oligonucleotide
aattcaaaaaggagggccagcttgtaaagatctcgagatctttacaagctggccctcc

SEQ ID NO: 619 - DNA, 58 bp, Artificial Sequence
  Description of Artificial Sequence Synthetic oligonucleotide
aattcaaaaatttagctgtaatgtctttactctcgagagtaaagacattacagctaaa

SEQ ID NO: 620 - DNA, 58 bp, Artificial Sequence
  Description of Artificial Sequence Synthetic oligonucleotide
aattcaaaaagggaactcctcctccatttgactcgagtcaaatggaggaggagttccc

SEQ ID NO: 621 - DNA, 58 bp, Artificial Sequence
  Description of Artificial Sequence Synthetic oligonucleotide
aattcaaaaatttgagcaacttgggaacaatctcgagattgttcccaagttgctcaaa

SEQ ID NO: 622 - DNA, 58 bp, Artificial Sequence
  Description of Artificial Sequence Synthetic oligonucleotide
aattcaaaaactctctttctctctctttcttctcgagaagaaagagagagaaagagag

SEQ ID NO: 623 - DNA, 58 bp, Artificial Sequence
  Description of Artificial Sequence Synthetic oligonucleotide
aattcaaaaatctctttctctctctttcttactcgagtaagaaagagagagaaagaga

SEQ ID NO: 624 - DNA, 58 bp, Artificial Sequence
  Description of Artificial Sequence Synthetic oligonucleotide
aattcaaaaattctctctctttcttaattgtctcgagacaattaagaaagagagagaa

SEQ ID NO: 625 - DNA, 58 bp, Artificial Sequence
  Description of Artificial Sequence Synthetic oligonucleotide
aattcaaaaatctctctctttcttaattgtactcgagtacaattaagaaagagagaga

SEQ ID NO: 626 - DNA, 58 bp, Artificial Sequence
  Description of Artificial Sequence Synthetic oligonucleotide
aattcaaaaactctctctttcttaattgtatctcgagatacaattaagaaagagagag

SEQ ID NO: 627 - DNA, 58 bp, Artificial Sequence
  Description of Artificial Sequence Synthetic oligonucleotide
aattcaaaaagctatgggctgtatttattgtctcgagacaataaatacagcccatagc

SEQ ID NO: 628 - DNA, 58 bp, Artificial Sequence
  Description of Artificial Sequence Synthetic oligonucleotide
aattcaaaaagctgtatttattgtgaatctactcgagtagattcacaataaatacagc

SEQ ID NO: 629 - DNA, 58 bp, Artificial Sequence
  Description of Artificial Sequence Synthetic oligonucleotide
aattcaaaaagcgcggtcaaagtgcaatgatctcgagatcattgcactttgaccgcgc

SEQ ID NO: 630 - DNA, 58 bp, Artificial Sequence
  Description of Artificial Sequence Synthetic oligonucleotide
aattcaaaaacgcggtcaaagtgcaatgattctcgagaatcattgcactttgaccgcg

SEQ ID NO: 631 - DNA, 58 bp, Artificial Sequence
  Description of Artificial Sequence Synthetic oligonucleotide
aattcaaaaattcagttcggttggctaaacactcgagtgtttagccaaccgaactgaa

SEQ ID NO: 632 - DNA, 58 bp, Artificial Sequence
  Description of Artificial Sequence Synthetic oligonucleotide
aattcaaaaatcctatctttgtatgttaagactcgagtcttaacatacaaagatagga

SEQ ID NO: 633 - DNA, 58 bp, Artificial Sequence
  Description of Artificial Sequence Synthetic oligonucleotide
aattcaaaaaatcaaccaacacaagatcaaactcgagtttgatcttgtgttggttgat

SEQ ID NO: 634 - DNA, 58 bp, Artificial Sequence
  Description of Artificial Sequence Synthetic oligonucleotide
aattcaaaaagtgcttcttacattataaagtctcgagactttataatgtaagaagcac

SEQ ID NO: 635 - DNA, 58 bp, Artificial Sequence
  Description of Artificial Sequence Synthetic oligonucleotide
aattcaaaaatttaaagaacccacaaactatctcgagatagtttgtgggttctttaaa

SEQ ID NO: 636 - DNA, 58 bp, Artificial Sequence
  Description of Artificial Sequence Synthetic oligonucleotide
aattcaaaaagaacccacaaactattatacactcgagtgtataatagtttgtgggttc

SEQ ID NO: 637 - DNA, 58 bp, Artificial Sequence
  Description of Artificial Sequence Synthetic oligonucleotide
aattcaaaaaacccacaaactattatacatactcgagtatgtataatagtttgtgggt

SEQ ID NO: 638 - DNA, 58 bp, Artificial Sequence
  Description of Artificial Sequence Synthetic oligonucleotide
aattcaaaaattaaatatcctgcacttgttactcgagtaacaagtgcaggatatttaa

SEQ ID NO: 639 - DNA, 58 bp, Artificial Sequence
  Description of Artificial Sequence Synthetic oligonucleotide
aattcaaaaagaatacgcatatcccttctttctcgagaaagaagggatatgcgtattc

SEQ ID NO: 640 - DNA, 58 bp, Artificial Sequence
  Description of Artificial Sequence Synthetic oligonucleotide
aattcaaaaatacgcatatcccttctttgttctcgagaacaaagaagggatatgcgta

SEQ ID NO: 641 - DNA, 58 bp, Artificial Sequence
  Description of Artificial Sequence Synthetic oligonucleotide
aattcaaaaaaggtcaatgccaacgaacaaactcgagtttgttcgttggcattgacct

SEQ ID NO: 642 - DNA, 58 bp, Artificial Sequence
  Description of Artificial Sequence Synthetic oligonucleotide
aattcaaaaatgaccctcaccagcattatcactcgagtgataatgctggtgagggtca

SEQ ID NO: 643 - DNA, 58 bp, Artificial Sequence
  Description of Artificial Sequence Synthetic oligonucleotide
aattcaaaaacgctcagcaagttggtagtttctcgagaaactaccaacttgctgagcg

SEQ ID NO: 644 - DNA, 58 bp, Artificial Sequence
  Description of Artificial Sequence Synthetic oligonucleotide
aattcaaaaaccctgtcgactgccttatacactcgagtgtataaggcagtcgacaggg

SEQ ID NO: 645 - DNA, 58 bp, Artificial Sequence
  Description of Artificial Sequence Synthetic oligonucleotide
aattcaaaaacccgtcccgtcaaacaagtttctcgagaaacttgtttgacgggacggg

SEQ ID NO: 646 - DNA, 58 bp, Artificial Sequence
  Description of Artificial Sequence Synthetic oligonucleotide
aattcaaaaagtcccgtcaaacaagtttcttctcgagaagaaacttgtttgacgggac

SEQ ID NO: 647 - DNA, 58 bp, Artificial Sequence
  Description of Artificial Sequence Synthetic oligonucleotide
aattcaaaaacgtcaaacaagtttcttcttactcgagtaagaagaaacttgtttgacg

SEQ ID NO: 648 - DNA, 58 bp, Artificial Sequence
  Description of Artificial Sequence Synthetic oligonucleotide
aattcaaaaaacgagtatctctatatatagtctcgagactatatatagagatactcgt

SEQ ID NO: 649 - DNA, 58 bp, Artificial Sequence
  Description of Artificial Sequence Synthetic oligonucleotide
aattcaaaaacgagtatctctatatatagtactcgagtactatatatagagatactcg

SEQ ID NO: 650 - DNA, 58 bp, Artificial Sequence
  Description of Artificial Sequence Synthetic oligonucleotide
aattcaaaaacgtctctgcactgagatacatctcgagatgtatctcagtgcagagacg

SEQ ID NO: 651 - DNA, 58 bp, Artificial Sequence
  Description of Artificial Sequence Synthetic oligonucleotide
aattcaaaaagtctctgcactgagatacatactcgagtatgtatctcagtgcagagac

SEQ ID NO: 652 - DNA, 58 bp, Artificial Sequence
  Description of Artificial Sequence Synthetic oligonucleotide
aattcaaaaatctgcactgagatacataagactcgagtcttatgtatctcagtgcaga

SEQ ID NO: 653 - DNA, 58 bp, Artificial Sequence
  Description of Artificial Sequence Synthetic oligonucleotide
aattcaaaaactgcactgagatacataagaactcgagttcttatgtatctcagtgcag

SEQ ID NO: 654 - DNA, 58 bp, Artificial Sequence
  Description of Artificial Sequence Synthetic oligonucleotide
aattcaaaaatgcactgagatacataagaaactcgagtttcttatgtatctcagtgca

SEQ ID NO: 655 - DNA, 58 bp, Artificial Sequence
  Description of Artificial Sequence Synthetic oligonucleotide
aattcaaaaaactgagatacataagaaacaactcgagttgtttcttatgtatctcagt

SEQ ID NO: 656 - DNA, 58 bp, Artificial Sequence
  Description of Artificial Sequence Synthetic oligonucleotide
aattcaaaaacataagaaacaagggtagtttctcgagaaactacccttgtttcttatg

SEQ ID NO: 657 - DNA, 58 bp, Artificial Sequence
  Description of Artificial Sequence Synthetic oligonucleotide
aattcaaaaacgcttctgttataccatctttctcgagaaagatggtataacagaagcg

SEQ ID NO: 658 - DNA, 58 bp, Artificial Sequence
  Description of Artificial Sequence Synthetic oligonucleotide
aattcaaaaactctccggcttctccattgaactcgagttcaatggagaagccggagag

SEQ ID NO: 659 - DNA, 58 bp, Artificial Sequence
  Description of Artificial Sequence Synthetic oligonucleotide
aattcaaaaatctccggcttctccattgaatctcgagattcaatggagaagccggaga

SEQ ID NO: 660 - DNA, 58 bp, Artificial Sequence
  Description of Artificial Sequence Synthetic oligonucleotide
aattcaaaaatctccattgaatggctaatgtctcgagacattagccattcaatggaga

SEQ ID NO: 661 - DNA, 58 bp, Artificial Sequence
  Description of Artificial Sequence Synthetic oligonucleotide
aattcaaaaactggatctttatctggcttacctcgaggtaagccagataaagatccag

SEQ ID NO: 662 - DNA, 58 bp, Artificial Sequence
  Description of Artificial Sequence Synthetic oligonucleotide
aattcaaaaactttgtccacactccggaatcctcgaggattccggagtgtggacaaag

SEQ ID NO: 663 - DNA, 58 bp, Artificial Sequence
  Description of Artificial Sequence Synthetic oligonucleotide
aattcaaaaatctctaaagaagtcaggaaagctcgagctttcctgacttctttagaga

SEQ ID NO: 664 - DNA, 58 bp, Artificial Sequence
  Description of Artificial Sequence Synthetic oligonucleotide
aattcaaaaagaagaagaggttcctcgaaagctcgagctttcgaggaacctcttcttc

SEQ ID NO: 665 - DNA, 58 bp, Artificial Sequence
  Description of Artificial Sequence Synthetic oligonucleotide
aattcaaaaaacttgggaacaatttggtaacctcgaggttaccaaattgttcccaagt

SEQ ID NO: 666 - DNA, 58 bp, Artificial Sequence
  Description of Artificial Sequence Synthetic oligonucleotide
aattcaaaaagcttgtctttctgtgccttacctcgaggtaaggcacagaaagacaagc

SEQ ID NO: 667 - DNA, 58 bp, Artificial Sequence
  Description of Artificial Sequence Synthetic oligonucleotide
aattcaaaaattcaacatctctcatcctatcctcgaggataggatgagagatgttgaa

SEQ ID NO: 668 - DNA, 58 bp, Artificial Sequence
  Description of Artificial Sequence Synthetic oligonucleotide
aattcaaaaacaagtttcttcttaggctaagctcgagcttagcctaagaagaaacttg

SEQ ID NO: 669 - DNA, 58 bp, Artificial Sequence
  Description of Artificial Sequence Synthetic oligonucleotide
aattcaaaaatttcagtgtcctagcggaatgctcgagcattccgctaggacactgaaa

SEQ ID NO: 670 - DNA, 58 bp, Artificial Sequence
  Description of Artificial Sequence Synthetic oligonucleotide
aattcaaaaacatcacattggagtcacaatcctcgaggattgtgactccaatgtgatg

SEQ ID NO: 671 - DNA, 58 bp, Artificial Sequence
  Description of Artificial Sequence Synthetic oligonucleotide
aattcaaaaaggacaatcagatagttcaaacctcgaggtttgaactatctgattgtcc

SEQ ID NO: 672 - DNA, 58 bp, Artificial Sequence
  Description of Artificial Sequence Synthetic oligonucleotide
aattcaaaaactgttgatgacgtgttctatcctcgaggatagaacacgtcatcaacag

SEQ ID NO: 673 - DNA, 58 bp, Artificial Sequence
  Description of Artificial Sequence Synthetic oligonucleotide
aattcaaaaaaggactcactgaaggagtttgctcgagcaaactccttcagtgagtcct

SEQ ID NO: 674 - DNA, 58 bp, Artificial Sequence
  Description of Artificial Sequence Synthetic oligonucleotide
aattcaaaaatgaaggagtttgtgcagtttgctcgagcaaactgcacaaactccttca

SEQ ID NO: 675 - DNA, 58 bp, Artificial Sequence
  Description of Artificial Sequence Synthetic oligonucleotide
aattcaaaaattcctatgcctgattccaaatctcgagatttggaatcaggcataggaa

SEQ ID NO: 676 - DNA, 58 bp, Artificial Sequence
  Description of Artificial Sequence Synthetic oligonucleotide
aattcaaaaatcctatgcctgattccaaatcctcgaggatttggaatcaggcatagga

SEQ ID NO: 677 - DNA, 58 bp, Artificial Sequence
  Description of Artificial Sequence Synthetic oligonucleotide
aattcaaaaaactcctccatcaccttcattcctcgaggaatgaaggtgatggaggagt

SEQ ID NO: 678 - DNA, 58 bp, Artificial Sequence
  Description of Artificial Sequence Synthetic oligonucleotide
aattcaaaaaagtcctggttcctctgataagctcgagcttatcagaggaaccaggact

SEQ ID NO: 679 - DNA, 58 bp, Artificial Sequence
  Description of Artificial Sequence Synthetic oligonucleotide
aattcaaaaaagcagccaagcagaaagaaacctcgaggtttctttctgcttggctgct

SEQ ID NO: 680 - DNA, 58 bp, Artificial Sequence
  Description of Artificial Sequence Synthetic oligonucleotide
aattcaaaaatgtcaagtagaggacagaaagctcgagctttctgtcctctacttgaca

SEQ ID NO: 681 - DNA, 58 bp, Artificial Sequence
  Description of Artificial Sequence Synthetic oligonucleotide
aattcaaaaaagaggacagaaagcaagaaagctcgagctttcttgctttctgtcctct

SEQ ID NO: 682 - DNA, 58 bp, Artificial Sequence
  Description of Artificial Sequence Synthetic oligonucleotide
aattcaaaaaacaaagcaaccagcacaattcctcgaggaattgtgctggttgctttgt

SEQ ID NO: 683 - DNA, 58 bp, Artificial Sequence
  Description of Artificial Sequence Synthetic oligonucleotide
aattcaaaaaatgggaactcctcctccatttctcgagaaatggaggaggagttcccat

SEQ ID NO: 684 - DNA, 58 bp, Artificial Sequence
  Description of Artificial Sequence Synthetic oligonucleotide
aattcaaaaatgggaactcctcctccatttgctcgagcaaatggaggaggagttccca

SEQ ID NO: 685 - DNA, 58 bp, Artificial Sequence
  Description of Artificial Sequence Synthetic oligonucleotide
aattcaaaaagtccttgtgcctctctctttcctcgaggaaagagagaggcacaaggac

SEQ ID NO: 686 - DNA, 58 bp, Artificial Sequence
  Description of Artificial Sequence Synthetic oligonucleotide
aattcaaaaactctctctttctctctctttcctcgaggaaagagagagaaagagagag

SEQ ID NO: 687 - DNA, 58 bp, Artificial Sequence
  Description of Artificial Sequence Synthetic oligonucleotide
aattcaaaaatctctctttcttaattgtatgctcgagcatacaattaagaaagagaga

SEQ ID NO: 688 - DNA, 58 bp, Artificial Sequence
  Description of Artificial Sequence Synthetic oligonucleotide
aattcaaaaagggctgtatttattgtgaatcctcgaggattcacaataaatacagccc

SEQ ID NO: 689 - DNA, 58 bp, Artificial Sequence
  Description of Artificial Sequence Synthetic oligonucleotide
aattcaaaaatttcagttcggttggctaaacctcgaggtttagccaaccgaactgaaa

SEQ ID NO: 690 - DNA, 58 bp, Artificial Sequence
  Description of Artificial Sequence Synthetic oligonucleotide
aattcaaaaattcctatctttgtatgttaagctcgagcttaacatacaaagataggaa

SEQ ID NO: 691 - DNA, 58 bp, Artificial Sequence
  Description of Artificial Sequence Synthetic oligonucleotide
aattcaaaaatttcgtcgttcgttctgtttcctcgaggaaacagaacgaacgacgaaa

SEQ ID NO: 692 - DNA, 58 bp, Artificial Sequence
  Description of Artificial Sequence Synthetic oligonucleotide
aattcaaaaaccacaaactattatacataagctcgagcttatgtataatagtttgtgg

SEQ ID NO: 693 - DNA, 58 bp, Artificial Sequence
  Description of Artificial Sequence Synthetic oligonucleotide
aattcaaaaataaatatcctgcacttgttagctcgagctaacaagtgcaggatattta

SEQ ID NO: 694 - DNA, 58 bp, Artificial Sequence
  Description of Artificial Sequence Synthetic oligonucleotide
aattcaaaaaatcctgcacttgttaggaatactcgagtattcctaacaagtgcaggat

SEQ ID NO: 695 - DNA, 58 bp, Artificial Sequence
  Description of Artificial Sequence Synthetic oligonucleotide
aattcaaaaatcctgcacttgttaggaatacctcgaggtattcctaacaagtgcagga

SEQ ID NO: 696 - DNA, 58 bp, Artificial Sequence
  Description of Artificial Sequence Synthetic oligonucleotide
aattcaaaaaacttgttaggaatacgcatatctcgagatatgcgtattcctaacaagt

SEQ ID NO: 697 - DNA, 58 bp, Artificial Sequence
  Description of Artificial Sequence Synthetic oligonucleotide
aattcaaaaacttgttaggaatacgcatatcctcgaggatatgcgtattcctaacaag

SEQ ID NO: 698 - DNA, 58 bp, Artificial Sequence
  Description of Artificial Sequence Synthetic oligonucleotide
aattcaaaaaggtcaatgccaacgaacaaacctcgaggtttgttcgttggcattgacc

SEQ ID NO: 699 - DNA, 58 bp, Artificial Sequence
  Description of Artificial Sequence Synthetic oligonucleotide
aattcaaaaaacttgaccctcaccagcattactcgagtaatgctggtgagggtcaagt

SEQ ID NO: 700 - DNA, 58 bp, Artificial Sequence
  Description of Artificial Sequence Synthetic oligonucleotide
aattcaaaaagctcagcaagttggtagtttcctcgaggaaactaccaacttgctgagc

SEQ ID NO: 701 - DNA, 58 bp, Artificial Sequence
  Description of Artificial Sequence Synthetic oligonucleotide
aattcaaaaaccgtcccgtcaaacaagtttcctcgaggaaacttgtttgacgggacgg

SEQ ID NO: 702 - DNA, 58 bp, Artificial Sequence
  Description of Artificial Sequence Synthetic oligonucleotide
aattcaaaaagtcaaacaagtttcttcttagctcgagctaagaagaaacttgtttgac

SEQ ID NO: 703 - DNA, 58 bp, Artificial Sequence
  Description of Artificial Sequence Synthetic oligonucleotide
aattcaaaaactctgcactgagatacataagctcgagcttatgtatctcagtgcagag

SEQ ID NO: 704 - DNA, 58 bp, Artificial Sequence
  Description of Artificial Sequence Synthetic oligonucleotide
aattcaaaaagcactgagatacataagaaacctcgaggtttcttatgtatctcagtgc

SEQ ID NO: 705 - DNA, 58 bp, Artificial Sequence
  Description of Artificial Sequence Synthetic oligonucleotide
aattcaaaaataagaaacaagggtagtttacctcgaggtaaactacccttgtttctta

SEQ ID NO: 706 - DNA, 58 bp, Artificial Sequence
  Description of Artificial Sequence Synthetic oligonucleotide
aattcaaaaagcttctgttataccatctttgctcgagcaaagatggtataacagaagc

SEQ ID NO: 707 - DNA, 58 bp, Artificial Sequence
  Description of Artificial Sequence Synthetic oligonucleotide
aattcaaaaactccggcttctccattgaatgctcgagcattcaatggagaagccggag

SEQ ID NO: 708 - DNA, 58 bp, Artificial Sequence
  Description of Artificial Sequence Synthetic oligonucleotide
aattcaaaaacttctccattgaatggctaatctcgagattagccattcaatggagaag

SEQ ID NO: 709 - DNA, 58 bp, Artificial Sequence
  Description of Artificial Sequence Synthetic oligonucleotide
aattcaaaaattctccattgaatggctaatgctcgagcattagccattcaatggagaa

SEQ ID NO: 710 - DNA, 58 bp, Artificial Sequence
  Description of Artificial Sequence Synthetic oligonucleotide
aattcaaaaaattgaatggctaatgtgtatgctcgagcatacacattagccattcaat

SEQ ID NO: 711 - DNA, 58 bp, Artificial Sequence
  Description of Artificial Sequence Synthetic oligonucleotide
aattcaaaaacagtcctggttcctctgataactcgagttatcagaggaaccaggactg

SEQ ID NO: 712 - DNA, 58 bp, Artificial Sequence
  Description of Artificial Sequence Synthetic oligonucleotide
aattcaaaaactgtaatgtctttactctttactcgagtaaagagtaaagacattacag

SEQ ID NO: 713 - DNA, 58 bp, Artificial Sequence
  Description of Artificial Sequence Synthetic oligonucleotide
aattcaaaaattgagcaacttgggaacaattctcgagaattgttcccaagttgctcaa

SEQ ID NO: 714 - DNA, 58 bp, Artificial Sequence
  Description of Artificial Sequence Synthetic oligonucleotide
aattcaaaaatgagcaacttgggaacaatttctcgagaaattgttcccaagttgctca

SEQ ID NO: 715 - DNA, 58 bp, Artificial Sequence
  Description of Artificial Sequence Synthetic oligonucleotide
aattcaaaaagagcaacttgggaacaatttgctcgagcaaattgttcccaagttgctc

SEQ ID NO: 716 - DNA, 58 bp, Artificial Sequence
  Description of Artificial Sequence Synthetic oligonucleotide
aattcaaaaactctttctctctctttcttaactcgagttaagaaagagagagaaagag

SEQ ID NO: 717 - DNA, 58 bp, Artificial Sequence
  Description of Artificial Sequence Synthetic oligonucleotide
aattcaaaaatctttctctctctttcttaatctcgagattaagaaagagagagaaaga

SEQ ID NO: 718 - DNA, 58 bp, Artificial Sequence
  Description of Artificial Sequence Synthetic oligonucleotide
aattcaaaaatttctctctctttcttaattgctcgagcaattaagaaagagagagaaa

SEQ ID NO: 719 - DNA, 58 bp, Artificial Sequence
  Description of Artificial Sequence Synthetic oligonucleotide
aattcaaaaaggtgggctatgggctgtatttctcgagaaatacagcccatagcccacc

SEQ ID NO: 720 - DNA, 58 bp, Artificial Sequence
  Description of Artificial Sequence Synthetic oligonucleotide
aattcaaaaaggctatgggctgtatttattgctcgagcaataaatacagcccatagcc

SEQ ID NO: 721 - DNA, 58 bp, Artificial Sequence
  Description of Artificial Sequence Synthetic oligonucleotide
aattcaaaaagcggtcaaagtgcaatgatttctcgagaaatcattgcactttgaccgc

SEQ ID NO: 722 - DNA, 58 bp, Artificial Sequence
  Description of Artificial Sequence Synthetic oligonucleotide
aattcaaaaactgaaggaaacgttcacatttctcgagaaatgtgaacgtttccttcag

SEQ ID NO: 723 - DNA, 58 bp, Artificial Sequence
  Description of Artificial Sequence Synthetic oligonucleotide
aattcaaaaaaccctggtgcttcttacattactcgagtaatgtaagaagcaccagggt

SEQ ID NO: 724 - DNA, 58 bp, Artificial Sequence
  Description of Artificial Sequence Synthetic oligonucleotide
aattcaaaaaccctggtgcttcttacattatctcgagataatgtaagaagcaccaggg

SEQ ID NO: 725 - DNA, 58 bp, Artificial Sequence
  Description of Artificial Sequence Synthetic oligonucleotide
aattcaaaaaggtgcttcttacattataaagctcgagctttataatgtaagaagcacc

SEQ ID NO: 726 - DNA, 58 bp, Artificial Sequence
  Description of Artificial Sequence Synthetic oligonucleotide
aattcaaaaattaaagaacccacaaactattctcgagaatagtttgtgggttctttaa

SEQ ID NO: 727 - DNA, 58 bp, Artificial Sequence
  Description of Artificial Sequence Synthetic oligonucleotide
aattcaaaaataaagaacccacaaactattactcgagtaatagtttgtgggttcttta

SEQ ID NO: 728 - DNA, 58 bp, Artificial Sequence
  Description of Artificial Sequence Synthetic oligonucleotide
aattcaaaaaagaacccacaaactattatacctcgaggtataatagtttgtgggttct

SEQ ID NO: 729 - DNA, 58 bp, Artificial Sequence
  Description of Artificial Sequence Synthetic oligonucleotide
aattcaaaaacccacaaactattatacataactcgagttatgtataatagtttgtggg

SEQ ID NO: 730 - DNA, 58 bp, Artificial Sequence
  Description of Artificial Sequence Synthetic oligonucleotide
aattcaaaaaatcccttctttgttgagtttactcgagtaaactcaacaaagaagggat

SEQ ID NO: 731 - DNA, 58 bp, Artificial Sequence
  Description of Artificial Sequence Synthetic oligonucleotide
aattcaaaaatcccttctttgttgagtttaactcgagttaaactcaacaaagaaggga

SEQ ID NO: 732 - DNA, 58 bp, Artificial Sequence
  Description of Artificial Sequence Synthetic oligonucleotide
aattcaaaaacccttctttgttgagtttaacctcgaggttaaactcaacaaagaaggg

SEQ ID NO: 733 - DNA, 58 bp, Artificial Sequence
  Description of Artificial Sequence Synthetic oligonucleotide
aattcaaaaacttgaccctcaccagcattatctcgagataatgctggtgagggtcaag

SEQ ID NO: 734 - DNA, 58 bp, Artificial Sequence
  Description of Artificial Sequence Synthetic oligonucleotide
aattcaaaaattgaccctcaccagcattatcctcgaggataatgctggtgagggtcaa

SEQ ID NO: 735 - DNA, 58 bp, Artificial Sequence
  Description of Artificial Sequence Synthetic oligonucleotide
aattcaaaaaagccctgtcgactgccttatactcgagtataaggcagtcgacagggct

SEQ ID NO: 736 - DNA, 58 bp, Artificial Sequence
  Description of Artificial Sequence Synthetic oligonucleotide
aattcaaaaaaggctaagaaacgcagtatatctcgagatatactgcgtttcttagcct

SEQ ID NO: 737 - DNA, 58 bp, Artificial Sequence
  Description of Artificial Sequence Synthetic oligonucleotide
aattcaaaaagctaagaaacgcagtatatacctcgaggtatatactgcgtttcttagc

SEQ ID NO: 738 - DNA, 58 bp, Artificial Sequence
  Description of Artificial Sequence Synthetic oligonucleotide
aattcaaaaagtatatacgagtatctctatactcgagtatagagatactcgtatatac

SEQ ID NO: 739 - DNA, 58 bp, Artificial Sequence
  Description of Artificial Sequence Synthetic oligonucleotide
aattcaaaaatacgagtatctctatatatagctcgagctatatatagagatactcgta

SEQ ID NO: 740 - DNA, 58 bp, Artificial Sequence
  Description of Artificial Sequence Synthetic oligonucleotide
aattcaaaaatctctgcactgagatacataactcgagttatgtatctcagtgcagaga

SEQ ID NO: 741 - DNA, 58 bp, Artificial Sequence
  Description of Artificial Sequence Synthetic oligonucleotide
aattcaaaaaataagaaacaagggtagtttactcgagtaaactacccttgtttcttat

SEQ ID NO: 742 - DNA, 58 bp, Artificial Sequence
  Description of Artificial Sequence Synthetic oligonucleotide
aattcaaaaaccgtcactgcgcttctgttatctcgagataacagaagcgcagtgacgg

SEQ ID NO: 743 - DNA, 58 bp, Artificial Sequence
  Description of Artificial Sequence Synthetic oligonucleotide
aattcaaaaacgtcactgcgcttctgttatactcgagtataacagaagcgcagtgacg

SEQ ID NO: 744 - DNA, 58 bp, Artificial Sequence
  Description of Artificial Sequence Synthetic oligonucleotide
aattcaaaaagtcactgcgcttctgttatacctcgaggtataacagaagcgcagtgac

SEQ ID NO: 745 - DNA, 58 bp, Artificial Sequence
  Description of Artificial Sequence Synthetic oligonucleotide
aattcaaaaatggctaatgtgtatgtgaaatctcgagatttcacatacacattagcca

SEQ ID NO: 746 - DNA, 58 bp, Artificial Sequence
  Description of Artificial Sequence Synthetic oligonucleotide
aattcaaaaaggctaatgtgtatgtgaaatactcgagtatttcacatacacattagcc

SEQ ID NO: 747 - DNA, 58 bp, Artificial Sequence
  Description of Artificial Sequence Synthetic oligonucleotide
aattcaaaaactttctctctctttcttaattctcgagaattaagaaagagagagaaag

SEQ ID NO: 748 - DNA, 58 bp, Artificial Sequence
  Description of Artificial Sequence Synthetic oligonucleotide
aattcaaaaagtgggctatgggctgtatttactcgagtaaatacagcccatagcccac

SEQ ID NO: 749 - DNA, 58 bp, Artificial Sequence
  Description of Artificial Sequence Synthetic oligonucleotide
aattcaaaaatgggctatgggctgtatttatctcgagataaatacagcccatagccca

SEQ ID NO: 750 - DNA, 58 bp, Artificial Sequence
  Description of Artificial Sequence Synthetic oligonucleotide
aattcaaaaagggctatgggctgtatttattctcgagaataaatacagcccatagccc

SEQ ID NO: 751 - DNA, 58 bp, Artificial Sequence
  Description of Artificial Sequence Synthetic oligonucleotide
aattcaaaaacctggtgcttcttacattatactcgagtataatgtaagaagcaccagg

SEQ ID NO: 752 - DNA, 58 bp, Artificial Sequence
  Description of Artificial Sequence Synthetic oligonucleotide
aattcaaaaactggtgcttcttacattataactcgagttataatgtaagaagcaccag

SEQ ID NO: 753 - DNA, 58 bp, Artificial Sequence
  Description of Artificial Sequence Synthetic oligonucleotide
aattcaaaaatggtgcttcttacattataaactcgagtttataatgtaagaagcacca

SEQ ID NO: 754 - DNA, 58 bp, Artificial Sequence
  Description of Artificial Sequence Synthetic oligonucleotide
aattcaaaaaggctaagaaacgcagtatatactcgagtatatactgcgtttcttagcc

SEQ ID NO: 755 - DNA, 58 bp, Artificial Sequence
  Description of Artificial Sequence Synthetic oligonucleotide
aattcaaaaagctaatgtgtatgtgaaataactcgagttatttcacatacacattagc

SEQ ID NO: 756 - DNA, 21 bp, Homo sapiens
cctggttcctctgataagatc

SEQ ID NO: 757 - DNA, 21 bp, Homo sapiens
gcccaacgggcacttaagttt

SEQ ID NO: 758 - DNA, 21 bp, Homo sapiens
cagcagccaagcagaaagaaa

SEQ ID NO: 759 - DNA, 21 bp, Homo sapiens
gtgcttcttacattataaagt

SEQ ID NO: 760 - DNA, 57 bp, Artificial Sequence
  Description of Artificial Sequence Synthetic oligonucleotide
ccggccggcttctctaaagaagtcactcgagtgacttctttagagaagccggttttt

SEQ ID NO: 761 - DNA, 16000 bp, Homo sapiens
gggtctcccagtcccagtcccacacactgcctcatccccactcacagtcaacgcccgcaacaccccgccacccgcagtctgtcgcgcagtgacagcctgtcacaagcaccgccgcacaccggcacaatctgtcgtccacgcacagtctcacacacaacctgtcacacacgcacacacacacacagcggcacagtcgcacacacagccagcctcacgcgatcacaccaagcgtcacagcttgtcacagccatagattcctcacaataaaaataacagctaccgactccagagcaattatgcctgggcagcgggtgggtggggggaaggcgggcatatgtgacctcctgtgaccctcccaacacgaggcaggtatgagtaacctgttttacagatgaggaaacagaggccgcaaggtcaagtgccaaggtcatggggctagaagcagggtggtggggattacccagccagattcccaggagtgagcaagacttaaacaggttcactccagcttttcatcccccttctggccacctgaggctcctccttctggccttcccatgggaattcccttcctattccccagttctaagaagcccagagcaagaaacaggaggaagaggcttcctgattccccgcttaagggtgttcaaactgtctgcccctggctgggaaaacagggccacgccctccccaccaaatgctacttggctccaagtggggtactgatgggcactgaaaaaccgatgggcagtgagggctatcagaggggcaactacaagtacagtagcactaggcttgagtgtctgatgtgtgagtggccatgtggacggctggagttggtccgcagtggtggcagcttgtggtcctttgggggatttggtggtagaacggggccgggggtcttcaaaggcagagaagtctcaggacttcaaaccaacagcctaaccctgtccacacaggtggagtggcccaccttgggcccgggggggcctccaggagccccctggcccaggcaggccccgcctccaccgccccaggggccctccgaggtccagggcgcttcctcctctgcaacccgcttttcccttgctttccccttctttgttcctcccccgcctgctcctggggcttcccccattggagttgcctcggccaaaaccaacagggctccaacgacatcccgccttggcctgccgggcgggcggccggccctgccccatggcccgctcaggggccagatccgggctcagctgggtccggacggcggccctcgcgcacgtggttactcccaaggcgtgcgcaagcactcgcacaccccctggccctgcggccccgaaggggacctaggcgcgctcatgcacgtccctgtctgacaccccagcccggcaactcggacgtgcgcacgcacataagcacgctctcgtatagttgctcccacctgggtccgcagcatgcccttgcccggcgcccccagtcgggatctcaggcaccgaaagcttatatgcgcacctaggcctgacatcccaacgacactggctggcgcacttacacctataacatgcccctatcgggcaccacatgggggtccctaacacaagcattcactcccctgctttccctccgatggagctttcgagcacaggtactcgagccctgcgccccaacggggagctccaggtgggtccgtgagcccgctctggtgcccacacacatggctccaggcccacgcgggtacacacctccccagcctgcacacgcgtgtttcacatgagtcacacacctgccaggccgctcagggcatctggcttcaaagcggcattcaggttgttgcgggagccggcagggaagggaggcctaattccacctcccccatccctctcggaacacccgcctccccatatgtccctggggcacaggtgggacatcccagaactgccgctgcccagtggcgcccctgcatgtgctatatttatatttgaaatttgtacctttatatatactttttatatgtaagaaccgctcccccctcttcccgctccacgtttgtacttggattttaccacccccactaggcttcgacccaaacctgcaaaaaccatttcgccaggcagggatgtagggggaggcgggagcagccctccccctctccactgcggacggacatcccacgttcttaaagaccggggaccccccctcccccaagggccggactgcagctccgaatcccgcagccccagaaacacaatcgcgggggtgggtgctggcggcgagacgttgctttttggtgtgtgtttgctgttaccgcttaaattaaccctgagtgacggcttgcaattcgccacaaagaggccatttaatgccccccaccaccccacctcgggagaaatttccacttgcccccgagtccagctcgggccgcacgctacccgccctccaggccttcgctccaggtgcgcccgggcggtggaggcgagttccctgcgcgcatcatggacttcaggagtgagggtgggtgggttttggaggggtgggagcagaggcaggggggtgtgccgggactccccagacgctccactgcccaatcatagctctttgatatcccagcccggctggagaaagttagatggtactccagcgtctcctccgggtccccggaagagagaagctgggctgcaccccatgttggcacgaacactccctccccagtgcctctgtgcattaggtcgcgtgcctgtacgtaccctacctgagaccgcccctccgccaagtgcgcccctcgacaggtgcccgtcgcccacaggctcctttcattgtagcctccctggaggttctcaaggcgcctggcatttcctggacccgctgcaactgactcaagactggggggagggggctcatggaggtagaggtggtggggaggtctccccagggcccaggcaggaggagggaaggtcgtgtgctttccatgtcgcgggcaaaatgggtgctgacggtcactgtcacttgcatcgcacactgaggggtgggagaggacagcacatgcctggaccgtgcaggaggcccagaaccggggcgcgaaggggccgcgggacacgagcgggccgtgcgcaaagcctggcgcgcgcgtgcgcagcgacgaaaaggcgggaagcggcgcttcgaggccccggatgtcagacgcgacctgtgtggcatgtaccaggctggctccgacgacggaaccgccatgggtgctgagtggagagtggggaccgcggaccccgggggcaggtgaggggaaactgggagcataggtctggctttcgaccgtttgtgccgccagagtcttctcaatgatccctcccaggcaccgtaggccctctagtgggtctgggtggcgtcctcccggcccctcgctgctcaagcgcggctccctcttgccggggcgcggcctgcccctcacagtgacccccccgacccaccccccaaaaagtgaccccgcgctggtgggcatggaggactgtcccagttcaccatttgctgcgtcaaaggccgccgccaaaaggggctccgatgtcggcatttttgccttaaggctagtttgtcccccaggcgcgcgggggttgggggttcagggccgcctgtgcctcagtttctctcctcctgcgcccatcctgacatccgacggaggataatgcgcgttggagggctttggccgtgaatgcgatgttgatgattgctgtgattctttgggtgcttgggagagtttggggtttaactgcccccaaatccgaggacagcttgggggcgtcccttcggagaggatctctccgcgtcgcatagtgagcgtcgtcatctctagactaccccggtttccgaggagcctcaccttcaatccccgagccttctcttttgggggagtaaaggcggggaaatttaccaggggagatttttgtttccagggttggctgggtttgggggattttgtcctccagcgagcgcggggaagaaatgtgactaagtggcagaaaatgcgctttccctttctctctcttctgccttcgggccgcggggagttgcgcagactctaaaaaaaaaacctttaaaaacccaaaacctcccctccaagtccctttcgatctacgccaccttccctctcagggctgatttcttgaatttggggcgtcgaggcctccccacccgttgctaccagcccttttgcagcacttcctgggcaggccaggggagaggacggaaaagtgaaaaaagaacgtgtgcgcccagcggacccgacttaacctgttggtggaggaggaagctggcgtcgggctgaagtcccccgagccacccctggagccagagcagcccccgagcattcagagaaagccgcccgtccccagtcccctcggaagccggtcataaacccgacttctcatcacccgaattacttaaatgagctttgatggtgttaggtccttttccacgtgcggatgactcagcccgctgcagctgcgcccggggaggtgaccctggggcagcggctgaccctgctgcgccctgtaccctggacccagacgcggcccatcccagcagcaccccccgccccaagctcccagaccacacttagctttttttttccccccttactcttcgagtctttttcctcttctctaaaaacattttttttaatttaaaggagttttattacttcttttttttttataaagcatgtgagacagaggctgaatggtagaaacaggctgttaaaaaaagaaaaatacgtgccggtgacgcttcgtggagctggcgagcctcgcctgtcccgcgactgagcttgcggtcgccccgattctctagtagaccctggcgtggggcacctgagatctccgccactgaaaaggcgctatagaaatgcaaggaattatgattttaataaaattgtctcatttaggagcgagcttctgcgctgcctctgtcgtcggagcccgggtttgccacggcaaagtctgtaatcagagaaaaataccagctttgttgagggttagaatatatcattggctaatgtgtgtggttttctttttaaataatgctaagcattggaataattaaagagtgagaaataactcgtctctcctctctcctctccattcagcattgtcttatttaagggcggaaagcgcgcttttcggcagagctggttagtttttaatgcgggctttttttttctcacccgccgttccctccgctccggtctgattcgcaaatccccaaactggcacaaaccgggaaacttgcggccggccgttcttctccccgagttggagacagccctctcctcttccctcccccgcctctgggccctgacccactgcctggtttgcgctagagccgtttaaaaaagaaagaaaggaagaaaggagccgggctctggtaccccgatgccagccgagggcgcgtttcccaggcggcgggctggtgggagagtcaaacccgggtgtggggaagcagcttcgctgcctactctgcatcctctcccctaaaaagaatcaagtatttctagagatcgcctgctcagagggctcagatcccttgtcatattttaaattaaattgaacagttctttctttccagccccatatgcaataggaagagaaaatgcagtcttggagagcccgtcttggcaaccctgtgcccttgatagaagagagagctgcccacttgggctgggcctggcatccagccccagaggagtgctcttgccactggccacagggcctggatttggggaggaagagcagggtccagggccagtggctgagcacggcctggagagatgtgggagttggggaaaggactttttatcagggctgcagaggttcggctctgggctggtggtagggggctggtggtaggcgaggtggctgcggtggcccttccgccaggtgtggggtagaccgcttagaagggaaaggggatcctgaagtgcctgaggaagaaacagcaatgagaaagcaatgggaaaaatgggcaataaatgggcataggggttagtgcccacaggcgctagtggctgtgaagtgcctttgaggtgcaaagggagctttttatgtatttgcttcaggtgtttttcattacatgtgtttcccttttattttccttgttatttggtccttagacttttcagcgatcccttgtattcgtctttttacataagcagaaacttttttttttcctccttagagagatactgtgggaggtattttgccttcagtgctttctctttcctgctttttttaaaatttaattttaaaattataaagtaaatttagtgctagtgaaagtgaggggggaaccagctggggctggagccagggaaggggtaggcagaggcagggttctgcctcccttcctgaggcagccccgccccaggaccctaggcccagcctggtgtcgcttgctcactctctctgctctcttctcttgatgccattctactcccctcaccttcctgcagggttcttgtggacagggatggggacagtgtatcccagtgggtgcccattctccctttgggaacccctggctctgcagagtcattttccttcatgacctacatactgaggccccccattccctgcctgccaggcaagcagggcgagaggaggggcccttgctcccctcagacactaaggctgtttggctttcagctcggcgggtcctgtttgtcccccatacatgccctgttagtctgctaaacccgctgcacagttggttgccctgttgatgggatcagagttgggcaggcatagagagatcacacacacacacacacacacacgcacacgcacacgcactctgcagcagtgaggatgagaggggtttctatttgccacacatttttgtggtcacatgaatagcacactctgctctataacacgtgtatgcacataggtgtttgcatttgatgatactggtgtgacaaggaggtatttctaagccatataaattatgagaactgtatagcttaaaaatttaaggcttgttcagcggtgctgaaaatgtttatttcttaatctgggtggggttacatgggtgtgttcactttgtgaaagtgtatcaagcggcctaacttacagtgtgtgcactttttatacctgccttgcttcagttaaaaagtttacttaaaaaaagaagccccactctcaaaggtaaagaaaaacctgtgcattacaaaatataataaaaatttaaataaaaatttcctccagcctgctttgcttagtccttcctgctaggaatggtgggagggcttgtaaaaaatagcgggctgatataaaagctagcaggcatgtggtccacgaggcatcctgacccgtccacatgtgtggagtgcctgaaaactgcctacagacaggtatagaggtaggattttaacatctcagggctaaaggataagaaatggtgaggagtaagttacttgccaattacattttgctgtcaacatctggaaagcaaaattgcagctgttctctctgcccctgccccccgcccccgcattgccacctcttcttttttttctgcacacttacattttttgtacgcttgccatttatacttcggttttctgcactgttaaaacaattccatggctctgttcaaattgggttgttgggtttgagttttttttttttttttaatggatcttgtagtctgacagtataatttttggagggatttatttaaacaggcccaggagccattgataatcccctcccttttctttcgccctctctgtcttcccagaactgctcaggccagcaatgagggggaggacgctcggcagcccttgccgggctccttcctttagcctctgctcctccgggcaggtactgacagcttcgccctccatggcccttctgtggtgtttttgttttctcttcgccccccaaaaatatttgaaccaaaaagcctcttttttttttcctccacccacagcaaactgaaaggactagtgaaccgtgattcaacagagcgagggcgcctcttttcttccttagcattaattaactcaagttcgttaggacgttgggtctgtcgcccacagttttctcttgtgtgtgctgaatcgggtcatagagggagacttcccgtgcaaacagtttgtgttggagctgaaggacgcagagggggagagagggtgagaaagaaagggagccggggagggacggagcgtggccgaggggtgggcatggtccgagctcctcaaagcacccccggtgtcattctctgcaggggctagacaccagggaaatgtggattcttgggtttcctgggcaatgatcctgataataaaatagttgcgggagcaatacattttgaggaattgaaaaatgacctctttctaactcctaaaattgtacattttggggttctggccactgcctgtggctgaagcaattgcaagaaagaggttccctcaggaccgaggggtgccaggccgatctgagggcaggtggtgaggagaaatccttccttgctccttgtctctcctgtggcgggggaaacagaactggtaaaaaatgaaggggggtgtctggactgatcctgatggagctcttgcagagggactgggaagacctctgccttttgggttttgtcaccttgccctgacccctccgaaggaggggatttgctgtggtgcctggggtgagttttcagcattttgccatttgctctggccttctctctcgacttccagatattttcacagtgtgctgggcacagacctcatcttcacgcgctggtctctctttgccttgccttttgtcactttttcagttcttcttcagaaagagttatgaagctggcctgtgcagttcttccctcttcccgggcccttggccaccatgaagtgacacttcccaagccggttaggtgtgtgaggctctgcacttcccttgcccccggaagcccttccctgtcatctgtccttcacgcacttcggaaggtttcatccccatgcagcctgccggctggagaagagagctttggccttcctggcttttacttctgtttggtgacacaaggaagtttcaagctaaataatgaattaaagagtaggatttttgtttcccattccttgtagaggatgagatgaaagtaaacaatttccagttgccttagagagaatagattgtctcccctaatatccccattcctaccctatattattctgtgccaagagggtaagggagatgtgcctggagcagaggagcatttggagccagaacgaggctgcctgttggcccgatcccaggccaccgctcctggtttgtagtctggaggagaaaaggaaggggcggggggggggggggggcgcggtttacaggagagaagtgtgcggcctggctgtgaggcagaccctgtcctagacccagcactggggtgtggggcgaatccattcagccagcatgaggacactggaaagaaactgcgcagagccgcctttcctgagcagaggaaaacaggcccaagtcagccaaaaacccgctttgtccaaattatccgagctccctcgcaaagtgcattttccggtttccattcctggagtttccagacttttggatgattttgttctgactacagagccccttctcgttggcccccaactgaggggaggccccaccctcctctaggataagcagcagggccccttccctggctgcccagtgctgccgctgcgtcatgtctgcagttcccttcccatctctggccgctgtctggccactgtcttcttgtggccttccacgaagcagcagtaatcttgggacaaggtgggagagctcagggttacgaagccggcttcttttggttcagaccaaggctgaattataaaattccaacaattgggcaaactaattggggccctgtttatggcctttttaatttcccatgtcctttcatacccaggctgcagatatgctttattaaaatggatactggaatatctggtcatttctgtgaagttttgtaagttatttgctctaattgcctgtagttacccagatcaacatgggcccaagtgtccaggatctgaaagaataaatctgggccttaaagccagcaggatgtggacacataggatttttttttttttaagataaagaaggctactaaaagtggccctgtcttaaaatatacaacttcttatgcttctagatgtttcactataggtaggtgcgggcaggttgcaaaacctatctcagtctgggaagattattttgtttccttgaagaatttttaacactgtatatgtgagtgaggtgctgtacatattttcctatttttcttcaaataatccatacaggctcagtggacaggtctgttgtgtagagtttttgaaaaccatgcatttcttctcatggagaagcagtcattttccttaactggtttttctttctctgccgcggtgcagagcccttttctgtcttcttatttttcttcctctgctgttctgtttccctccatctttgtccttctttcttttcagttgtgggcctagcttggcactctctctccaggtttggcttttttctcttttttcgttctccttctctctatttctctttctccgtggccctttcagtctttttctgttgggggtacatttctcctagtgatggctgaaactggcatgggtatgtattttagaaatgtttcagtgaaaactgttgctggttttttttttgtttgtttgttttttttttttttttaccaaatagtactcaacaataaaagacagaggcttttcttccataatggcagcaaaatccctttccacaaaccaaaaacccatatgaatttttaaaaagtcttatttctggcttctcgatgatttttctctccaatgggggaaatacctttcccccacaatgtttcctgcttttccttttttccccccttgtcttttttttttctcttcctattaccagcatcagtggcttcctttccccatttcccatgtccttctactctgtggcctgcatgtccccttgctctttccatcacctgaactcttacacttgccagtgactcagacctggagaagccttgaatcgtatgagggattttttgtcttgattctggtctttggtgcaggatgtaaagcggtctgatctcctgcctggggctggaggggagaagagaaggaggctgggctgggaacaggtaatcttccaacaggagcctccactctttaacaaaagggggacagtttccttttgtctagggctcatgaggggcgtgtctgggctgatggccgccaccagtggcatgcaggaggagggggcccgcagtgaagggtgcttgaaatgtttctaatggaacccgagcaattcctctgtgtgcgcccaaaataggaggggaggaagggagggagggaggaaagagaaatagagaaagagaccttctcatctcccgaagcatcagaatatcctggccactcctccagaaaggcctcctccactgggagccagcagcccatagagagaatggggtgaggggaaagtatttgcctgagaatcttctgcccacctgcaaggttatagccactggcaggagaaggacaattgacaattgttcttcctccaactttctgccctgggaaaggcctgttgcttcttctgttaagagagccttgagggggtgcttggggctctttgtaggtccctgaggctctattgagacactcttctgccgcctctcctctgatctctccgccagtccgtggagttttgacccccaacccctctccaggagccagtcgtgcatcttgtcctgtttccctaacttctacatggatccttccatctgtcatttcccagctgtgggactgttgctgcccccacctctatccctgacccagttgtaccaccagattcagattcaacccatgtcgagcatgtctcttctactgtggcactgtggtctgtgaagtggaacatgccaggattctccaggaggctcgtgttggccattttctgtttggtgtctaatatttagtataaatcagaatacaagaaaaacacctccatgccaaatataaactcacttgtagtttttaatgacctcatcttgactttagaaggtaaaattgtttccacaaggtggagaggcaggatttggtaccttatgtcatgtctctgagctaacatctttggctgattggactgaagctggttattaacatgatgtgaattattggacaccaccctaagaggcctgtctctgataccacctgccttctgtactctcacctctaaccgtttctttcagggatcagccatgagctcagttcacttgagatggataattcagggcacagactggggtcccaggtgttgggcagcccctctcccattgtcagggttcagcgccagcccagggctggatgttttattttggcatgccccaggtgctcttccgggaacctttctgccaacataaaggccacaaagcacagggtttcagttggggaagaaaggggcacgagtgagcagtgaccttttttccgtcgccagctgaggactgagcctcgctgagggagagggcggctgtagggatggggttctgagtggaattggatccctttctccctgtctgcagccacatccttcttcctctgcttttccttttatgtcatatctatttccttcttactgtgtttcgaaggcctcaagcatggctgctccatagctctaactggagagaccccctcagaagtgggactcccctacccagtgctcaccctttctgcttgctgtgaggaaggtccccaagtggaggaccagagcaggggctccccggtgggtcacttctctgtgacttgatttcccccaggtggcctgtttttgatagagccctttgctgttttttgttgtgtcctttcttagctcttttcttttcatttgggtttctcattttatacttttgcactttgcactgctagggtttgggctctttagtctaaattgatttgggagacaggtaagaaaactaatctcaggtcaactggagagggcagatctggagaattaaatgcctgtgcagtggtgaggggcgttggtcagattccttgcctctgctcagaggtagtgtccatctttttgcctttgtcacttctccccgggtctggctgttgtttgtggctgcgtgtagagcttgtcaggcggagtgctgtgacaggaggctattttaataaactgttgcagtttctcgagccgagctgtgttgcgttggcatccctcgtcaccgctaatggagtgtgcgtggctgggtgtgggccgaaggaggaggctgagcttgctgggggctgggggagaagggagggacgtgtggggtgctggcctcccccttgtctgagagtgctccttcagcagccagacctgggggcatcacaaggtggcagtaccctgggcaccagatccctgccctggaagtactgctggcatggacactttctgagctctctttcttcttatttctattcatagcaccgcaggcccgtgtgtatgtggccggggaggaaaacttccactcgcccctgtgtgctccataggaagaaaaaaaaaaagaaagaaagaaatagaaacagaaaaagagagtgtgcaggcccgagagaaagactgaaaccaaaacacagagagaaggcgcagctgcttccccaaggccttctggggctcccgcccgccagcccgccgggagtcaggcctttatttatttatttttcaggcttaaaaaaaaatttcgagtcttccacaatcccagtcccccccaatgccccaccccacccccagatttgctgggtggctgctgccaaatggacccgagtgggagcctggaatgaaaaattcataactgctggactttgcttgttcattagacgtgaacttgtcgattgggcaaattgtttttggtctccaactgccgggggctctccccaccctccctgctcgcccggttccctcctccccttggacgcagccattggctgctcgtggatgtctctttgccaaataggtggatccttctctctctttctctctctctctctctctctctctctgtctctttctccccccacccctttttactggcttggcacaagcaaatggatggggattgagcctgaaaggagagagagagagggagtttgagagagagaaaaggagcaaaaaaaaaaaacaccccaaaaacccaaccagtgcgcacacacacgcgcacactcacacacacgccccatcccatccacgtcctccctcgatcctcgatctctccctcccccccttcttcctttcctccctccctcgctccctctctctcttttgcacgcgtctgccagcaacggtctgcagccggtcagaactcgtcctcttccccgggaatctgcgagctcccccttttcctccgatcaggcagctcgaagtttacacccctgtgccgctgccaaagccgaaagcctttttcttcagctgccgctttttccctcctgggttttgtttttgtttttgttttgcacgggggtggggtggggtgcgttgttggttgtggggagatggtgggaggctggttttgatttttaaattttgcatttttttctttttttttttttttaaactggaagaggatgcacaggggaagaaattgaaaaaaaaattttgttggcttttgtttacctggcgtgtgtggcagccggctcgctccctctctctgcttgctatccctgacctttctttctttttgctccttttcaaaaaaaatattaatttcccccttctgtgcaatggagcatggggggggggaggagggggaagggtttgagaatccacccaagcccggcccctattccccagaacaccaataataaccccctttaaaacatttaccttcctcccctgctcctcctcctcccccctccccccacccgcccccaactcacaactcttttgagtccagaatctcagaatcgggcgttgggctttgccgggtgcttcagatcaatggtaattatttaattttttccagttttatttttgtaaacagaaatcaattattatttaaacttcaaacaagcaaacaaccaaaaaaaaaaaaaaaaaccaaacaaaaccgagagagcccatccttctgtcacctgactgagtgggaaaaagggtgaaggggttgtgggaggctggggaaggggtcgcaagaagacccatgtagcttttaaccctaatgtggccgagacaagcaccttatttgtgctaacaagaagtgttttgttcattattactgtgattaacattagtattggtgtcgataacaaagctgaaatcacatatttaggatttaggtctgattaaaaaatgttggggtggatgttccaactggatcaggagaaaagaaaatgaaaacagcctggggagagggaagcctgatctgtttcctcactcgcttgctcgtggatgtcattttctgtcttcttgggggcggccaaaaatcgaccggtgtcggggaccagaggcggccccgcacgcccccgcgtgtgcgtccacgggcgtctgtgcagacggacactgtgccggggcgagctgacaggagttcacggctgcgatagaacatggagatgtcatgggcgcgacagagcctggcggggataccagcagcgtgtgtgtgtggacggcaacgttgtctgtgcgcgtgtgtgtgagtgagtgagggagagagagagagaataggtgtgtgtagaggctcccggtgcctctgtctggctgctgaggctgagatgggagcaagtggctggcgaagctggtggtggcttcaaaccacactttcgtagaacaatcgcaagagaaaattgttggggggagggaggaggaggagaaggcggttttccttgtgcccccccttctaacgctgcttttctccttctctctttccccctcatcccgtcttcccctcctcccgtcctccctcgccccgcatg

SEQ ID NO: 762 - DNA, 58 bp, Artificial Sequence
  Description of Artificial Sequence Synthetic oligonucleotide
ccggcctgttgatgacgtgttctatctcgagatagaacacgtcatcaacaggtttttg

SEQ ID NO: 763 - DNA, 58 bp, Artificial Sequence
  Description of Artificial Sequence Synthetic oligonucleotide
ccggcacatcacattggagtcacaactcgagttgtgactccaatgtgatgtgtttttg

SEQ ID NO: 764 - DNA, 57 bp, Artificial Sequence
  Description of Artificial Sequence Synthetic oligonucleotide
  modified_base (5)..(25): a, c, t, g, unknown or other
  modified_base (32)..(52): a, c, t, g, unknown or other
ccggnnnnnnnnnnnnnnnnnnnnnctcgagnnnnnnnnnnnnnnnnnnnnnttttt